(19) United States
(12) Patent Application Publication
(10) Pub. No.: US 2026/0277101 A1
(43) Pub. Date: Sep. 17, 2026

(54) RESIST COMPOSITION, LAMINATE, AND PATTERNING PROCESS

(71) Applicant: SHIN-ETSU CHEMICAL CO., LTD., Tokyo (JP)

(72) Inventors: Satoshi KUSAMA, Joetsu-shi (JP); Kousuke OHYAMA, Joetsu-shi (JP); Shun KIKUCHI, Joetsu-shi (JP); Ryo NISHIKAWA, Joetsu-shi (JP); Masaki OHASHI, Joetsu-shi (JP)

(73) Assignee: SHIN-ETSU CHEMICAL CO., LTD., Tokyo (JP)

(21) Appl. No.: 19/556,138

(22) Filed: Mar. 4, 2026

(30) Foreign Application Priority Data
Mar. 7, 2025 (JP) 2025-036579

(51) Int. Cl. G03F 7/004; C07D 517/22

(52) U.S. Cl. CPC G03F 7/0045; C07D 517/22

(57) ABSTRACT

The present invention is a resist composition containing a hypervalent iodine compound represented by the following general formula (1) and a solvent. This can provide: a resist composition that can achieve high sensitivity and high resolution in a patterning process; a laminate including a resist film obtained from the resist composition; and a patterning process using the resist composition.

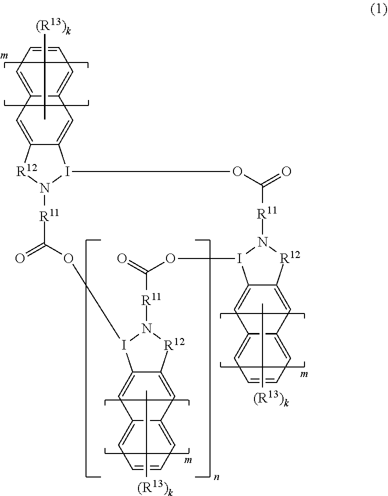

### TECHNICAL FIELD

The present invention relates to: a resist composition; a laminate; and a patterning process.

### BACKGROUND ART

While a higher integration density, higher operating speed and lower power consumption of LSIs are demanded to comply with the expanding IoT market, the effort to reduce the pattern rule is in rapid progress. In particular, logic devices drive forward the miniaturization technology. As the advanced miniaturization technology, devices of 10-nm node are manufactured in a mass scale by the double, triple or quadro-patterning version of the immersion ArF lithography. Furthermore, the experimental mass-scale manufacture of 7-nm node devices by the next-generation extreme ultraviolet ray (EUV) lithography of wavelength 13.5 nm has started.

As miniaturization advances, image blurs due to acid diffusion are regarded as a problem (Non Patent Document 1). In order to ensure resolution for fine patterns with a post-45 nm processed size, it is suggested that not only the enhancement of dissolution contrast, which has been proposed previously, but also the controlling of acid diffusion is important (Non Patent Document 2). In chemically amplified resist compositions, however, the sensitivity and the contrast are enhanced by acid diffusion. Accordingly, an attempt to minimize acid diffusion by lowering the temperature of post-exposure baking (PEB) and shortening the PEB time lowers the sensitivity and contrast markedly.

It is effective to control the acid diffusion by adding an acid generator that generates a bulky acid. Accordingly, it has been proposed to copolymerize a polymer with an acid generator in the form of an onium salt having polymerizable olefin. In post-16 nm processed size patterning of resist films, however, it is considered that patterning is impossible with chemically amplified resist compositions in view of the acid diffusion. Accordingly, development of a non-chemically amplified resist composition is desired.

Examples of materials for a non-chemically amplified resist composition include polymethyl methacrylate (PMMA). PMMA is a positive resist material whose solubility in an organic solvent developer increases due to decreased molecular weight caused by scission of the main chain by EUV irradiation.

Hydrogensilsesquioxane (HSQ) is a negative resist material which turns insoluble in an alkaline developer through crosslinking by condensation reaction of silanol generated by EUV irradiation. Calixarene substituted with chlorine also functions as a negative resist material. These negative resist materials have a small molecular size prior to crosslinking and are free from causing blurs due to acid diffusion, and therefore, exhibit smaller edge roughness and very high resolution.

Accordingly, the materials have been used as a pattern transfer material to show the resolution limit of the exposure apparatus. These materials, however, are insufficient in sensitivity, and further improvement is required.

The number of photons in EUV exposure being small is a factor that causes difficulties in developing materials for EUV lithography. The energy of EUV is much higher than that of an ArF excimer laser beam, and the number of photons in EUV exposure is 1/14 of that of ArF exposure. Furthermore, the size of the pattern formed by EUV exposure is half of that in ArF exposure or less. Therefore, EUV exposure is easily affected by variation in the number of photons. The variation in the number of photons in a radiation light region of extremely short wavelengths is the physical phenomenon of shot noise, and it is impossible to eliminate the influence of the variation. Therefore, so-called probability theory (stochastics) is attracting attention. The influence of shot noise cannot be eliminated, but there is discussion of how to reduce this influence. Due to the influence of shot noise, not only are critical dimension uniformity (CDU) and line width roughness (LWR) increased, a phenomenon that a hole gets blocked at a probability of one to several millions is observed. If a hole gets blocked, conduction failure occurs and the transistor does not function, and the performance of the entire device is adversely affected. Considering sensitivity in practical terms, resist compositions that mainly contain PMMA or HSQ are greatly affected by stochastics, and cannot achieve the desired resolution performance.

The introduction of an element that greatly absorbs EUV light is attracting attention as a means for reducing the influence of shot noise on the side of the resist. Patent Document 1 proposes a chemically amplified resist composition containing iodine atoms, which greatly absorb EUV light. However, as stated above, a chemically amplified resist composition cannot realize excellent resolution performance in EUV lithography, in which the processed size is to be further miniaturized in the future.

Patent Document 2 proposes a negative resist composition containing a tin compound. This composition mainly contains the element tin, which greatly absorbs EUV light, and therefore, stochastics is improved, and high sensitivity and high resolution can be realized. However, such a so-called metal resist has many problems such as insufficient solubility in a solvent for resists, storage stability, and defects due to residues after etching.

On the other hand, Patent Document 3 proposes a positive resist composition containing a hypervalent iodine compound. This composition contains the element iodine, which greatly absorbs EUV light, and therefore, stochastics is improved in the same manner as metal resists, and high sensitivity and high resolution can be realized. Furthermore, since the compound is constituted only by organic molecules, it is possible to improve solubility in developers and defects due to residues, which are issues of metal resists. However, performance as a resist material is still unsatisfactory, and there are demands for the development of a resist material useful for even finer patterning.

### CITATION LIST

### Patent Literature

Patent Document 1: JP2018-005224A
Patent Document 2: JP2021-503482A
Patent Document 3: JP2023-167368A

### Non Patent Literature

Non Patent Document 1: SPIE Vol. 5039 p1 (2003)
Non Patent Document 2: SPIE Vol. 6520 p65203L-1 (2007)

### SUMMARY OF INVENTION

### Technical Problem

The present invention has been made in view of the above-described circumstances, and an object thereof is to provide: a resist composition that can achieve high sensitivity and high resolution in a patterning process; a laminate including a resist film obtained from the resist composition; and a patterning process using the resist composition.

### Solution to Problem

To achieve the object, the present invention provides a resist composition comprising a hypervalent iodine compound represented by the following general formula (1) and a solvent,

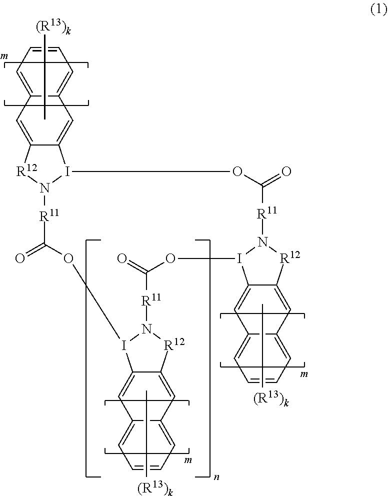

wherein “m” represents an integer of 0 to 2, “n” represents an integer of 1 to 40, and “k” represents an integer of 0 to 2m+4, the “m's” and the “k's” being identical to or different from each other;
each R¹¹ represents a carbonyl group or a hydrocarbylene group having 1 to 40 carbon atoms and optionally containing a heteroatom, the R¹¹'s being identical to or different from each other;
each R¹² represents a carbonyl group or a hydrocarbylene group having 1 to 10 carbon atoms and optionally containing a heteroatom, the R¹²'s being identical to or different from each other; and
each R¹³ represents a halogen atom or a hydrocarbyl group having 1 to 40 carbon atoms and optionally containing a heteroatom, the R¹³'s being identical to or different from each other.

The inventive resist composition as described has iodine atoms, which have a high ability to absorb EUV light. Furthermore, the resist composition used in the present invention contains a hypervalent iodine compound represented by the general formula (1) and a solvent, and a carboxylic acid compound, which is essential in Patent Document 3, does not need to be contained. As a result, the inventive resist composition can form a resist film having a uniform distribution of materials and also having a high density, and LWR and resolution performance can be even more improved from conventional non-chemically amplified resists. That is, because of these characteristics, the inventive resist composition can achieve high sensitivity, high resolution, and low LWR in a patterning process.

The present invention also provides a laminate comprising:
a substrate; and
a resist film obtained from the above-described resist composition on the substrate.

In the inventive laminate, including a resist film obtained from the inventive resist composition, the resist film has high sensitivity, exhibits excellent limiting resolution, is effective for precise fine processing, and moreover, can be applied to both positive and negative patterning. Therefore, the inventive laminate has a wide range of uses, and is highly useful in resist process technology.

In this case, the laminate may further comprise a resist underlayer film between the substrate and the resist film.

When a resist underlayer film is necessary for pattern formation, a resist underlayer film can also be disposed appropriately between the substrate and the resist film.

The present invention also provides a patterning process comprising the steps of:
forming a resist film by using the above-described resist composition on a substrate or on a resist underlayer film of a substrate on which the resist underlayer film has been laminated;
exposing the resist film by using a high-energy beam; and
developing the exposed resist film by using a developer.

According to the inventive patterning process as described, high sensitivity, high resolution, and low LWR can be achieved, since the inventive resist composition is used.

In the present invention, the high-energy beam used can be an i-line, a KrF excimer laser beam, an ArF excimer laser beam, an electron beam, or an extreme ultraviolet ray.

As the high-energy beam, various beams, such as those given above, can be used.

In the present invention, the developer used can be one that dissolves exposed portions and does not dissolve unexposed portions.

Alternatively, the developer used can also be one that dissolves unexposed portions and does not dissolve exposed portions.

In this manner, the inventive patterning process can be applied to both positive and negative patterning.

### Advantageous Effects of Invention

As described above, the inventive resist composition can achieve both high sensitivity and high resolution in a patterning process, for example, photolithography using a high-energy beam, especially electron beam (EB) lithography and extreme ultraviolet ray (EUV) lithography. Therefore, the inventive resist composition is extremely useful in forming a fine pattern.

In addition, the inventive laminate can be applied to both positive and negative patterning, and has a wide range of uses and is highly useful in resist process technology.

Furthermore, the inventive patterning process can achieve both high sensitivity and high resolution.

### BRIEF DESCRIPTION OF DRAWINGS

FIG. 1 is a schematic cross-sectional view showing an example of the inventive laminate.

### DESCRIPTION OF EMBODIMENTS

As stated above, there have been demands for the development of a resist composition that can achieve high sensitivity and high resolution in a patterning process.

The present inventors have earnestly studied the problem and found out that a resist composition mainly containing a predetermined hypervalent iodine compound can give a resist film that has extremely high sensitivity and exhibits excellent resolution, and is extremely effective for precise fine processing. Thus, the present invention has been achieved.

That is, the present invention is a resist composition comprising a hypervalent iodine compound represented by the following general formula (1) and a solvent,

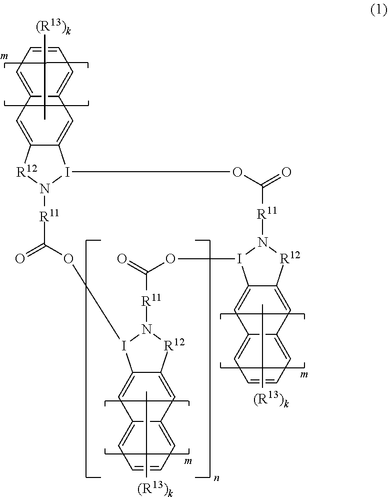

wherein “m” represents an integer of 0 to 2, “n” represents an integer of 1 to 40, and “k” represents an integer of 0 to 2m+4, the “m's” and the “k's” being identical to or different from each other;
each R¹¹ represents a carbonyl group or a hydrocarbylene group having 1 to 40 carbon atoms and optionally containing a heteroatom, the R¹¹'s being identical to or different from each other;
each R¹² represents a carbonyl group or a hydrocarbylene group having 1 to 10 carbon atoms and optionally containing a heteroatom, the R¹²'s being identical to or different from each other; and each R¹³ represents a halogen atom or a hydrocarbyl group having 1 to 40 carbon atoms and optionally containing a heteroatom, the R¹³'s being identical to or different from each other.

Hereinafter, the present invention will be described in detail, but the present invention is not limited thereto.

### [Resist Composition]

The inventive resist composition mainly contains a hypervalent iodine compound represented by the following general formula (1) and a solvent.

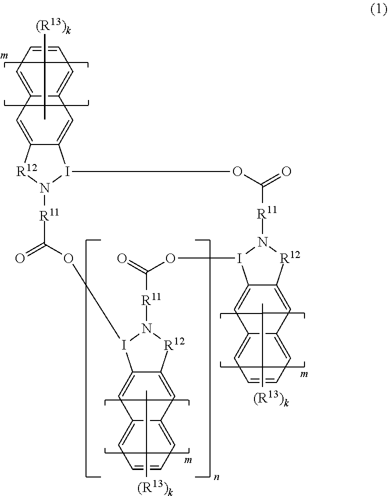

In the formula, “m” represents an integer of 0 to 2, “n” represents an integer of 1 to 40, and “k” represents an integer of 0 to 2m+4, the “m's” and the “k's” being identical to or different from each other;
each R¹¹ represents a carbonyl group or a hydrocarbylene group having 1 to 40 carbon atoms and optionally containing a heteroatom, the R¹¹'s being identical to or different from each other;
each R¹² represents a carbonyl group or a hydrocarbylene group having 1 to 10 carbon atoms and optionally containing a heteroatom, the R¹²'s being identical to or different from each other; and
each R¹³ represents a halogen atom or a hydrocarbyl group having 1 to 40 carbon atoms and optionally containing a heteroatom, the R¹³'s being identical to or different from each other.

The inventive resist composition mainly contains a hypervalent iodine compound represented by the general formula (1) as described above. On the other hand, the inventive resist composition does not need to contain a polymer or a photo-acid generator, which are contained in conventional chemically amplified resist compositions. However, in the inventive resist composition, a difference in solubility occurs between exposed portions and unexposed portions, particularly by virtue of EB or EUV exposure, and a positive or negative pattern can be formed. The mechanism is not completely clear and it is not desirable to be bound by the theory, but the following conjecture can be made, for example.

The hypervalent iodine compound represented by the general formula (1) is a cyclic oligomer including a tricoordinate hypervalent iodine compound. Such a hypervalent iodine compound has a comparatively large molecular size, and therefore, a homogeneous resist film can be formed on a substrate even when the composition does not contain a carboxylic acid compound, which is an essential component in Patent Document 3.

The resist film of the present invention formed on a substrate changes in polarity by the hypervalent iodine compound, being the main component of the resist film, being decomposed by light, and a pattern is formed by a development process. Incidentally, by selecting the developer appropriately, a positive or negative pattern can be formed.

The inventive resist composition can be either a positive type or a negative type depending on the choice of components. In the case of a positive type, by the hypervalent iodine compound represented by the general formula (1) being decomposed due to light, a monovalent iodine compound is formed, and at the same time, the oligomer structure is resolved, and the molecular weight is reduced. It is conjectured that, as a result, a positive pattern, where exposed portions are removed with an organic solvent, is formed.

On the other hand, in the case of a negative type, by the hypervalent iodine compound represented by the general formula (1) being decomposed due to light, exchange of cross links or bonds occurs, and increase in molecular weight and polarity conversion occur. It is conjectured that, as a result, a negative pattern, where unexposed portions are removed with an aqueous alkaline solution, is formed.

From the above-described conjecture, it can be said that the inventive resist composition is a non-chemically amplified resist composition. The inventive resist composition does not require an acid-labile group-containing base polymer or a photo-acid generator, unlike conventional chemically amplified resist compositions. Therefore, adverse effects (e. g. image blurs) due to acid diffusion do not occur, and resolution of a fine pattern is possible.

The inventive resist composition is extremely effective, especially in EUV lithography. This is because the inventive resist composition has iodine atoms, which are capable of greatly absorbing EUV light, and does not need to contain a carboxylic acid compound, which is essential in Patent Document 3, and therefore, it is possible to form a resist film having a uniform material distribution and also having a high density, and there is a characteristic that LWR and resolution performance can be even more improved from conventional non-chemically amplified resists. That is, the inventive resist composition can achieve high sensitivity, high resolution, and low LWR in a patterning process by virtue of these characteristics.

As a resist composition for EUV lithography with which a fine pattern can be formed, a metal resist that mainly contains a compound of tin, which is a metal having a high absorbance of EUV light in the same manner as iodine atoms (e. g. Patent Document 2), is reported. However, as described above, such a metal resist has many issues such as insufficient solubility in a solvent, storage stability, and defects due to residues after etching caused by a metal element being contained. On the other hand, the inventive resist composition has an advantage over metal resists regarding defects, since a metal element is not used, and there are no problems regarding solubility in a solvent either. Moreover, the inventive resist composition is applicable in the case of either a positive type or a negative type, and therefore, has a wide range of uses. For example, in a contact hole formation process, a reversal process step is necessary after forming a pillar pattern in the case of a metal resist performed with negative development, but such a step is unnecessary in the case of a positive resist. Therefore, it can be said that the inventive resist composition is more useful than metal resists from the viewpoint of the simplicity and convenience of the process as well.

JP2015-180928A and JP2018-095853A disclose a resist composition containing a hypervalent iodine compound as an additive and a resist composition in which a hypervalent iodine compound is incorporated in the polymer skeleton of a base polymer. However, the only characteristic of the resist compositions disclosed in these Patent Documents is improvement of line edge roughness, and there is no mention whatsoever regarding the possibility that the hypervalent iodine compound may be photolysed, or the possibility that the hypervalent iodine compound may function as a material for a non-chemically amplified resist composition. Furthermore, according to the description regarding the contained amount and specific examples, the hypervalent iodine compound is not the main component. Meanwhile, Patent Document 3 proposes a positive resist composition containing a hypervalent iodine compound, but there is no description regarding the inventive hypervalent iodine compound represented by the general formula (1), and there is no mention whatsoever regarding advantageous effects that, when an oligomer of a hypervalent iodine compound is used like in the present invention, a carboxylic acid compound, which is essential in Patent Document 3, does not need to be contained, it is possible to form a resist film having a uniform distribution of materials and also having a high density, and LWR and resolution performance can be even more improved from conventional non-chemically amplified resists. Accordingly, it is considered that a non-chemically amplified resist composition that is extremely highly sensitive, exhibits excellent resolution, and is extremely effective for precise fine processing like the inventive composition cannot be conceived from these Patent Documents. That is, it can be said that the present invention provides a clearly novel resist composition and a clearly novel patterning process.

In the following, each component of the inventive resist composition will be described.

### [Hypervalent Iodine Compound]

The hypervalent iodine compound represented by the following general formula (1) is a cyclic oligomer of a tricoordinate hypervalent iodine compound.

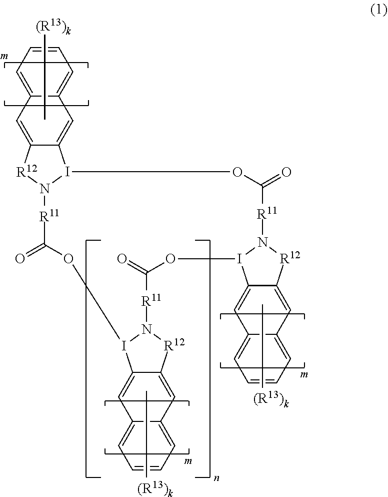

In the formula, “m” represents an integer of 0 to 2, “n” represents an integer of 1 to 40, and “k” represents an integer of 0 to 2m+4, the “m's” and the “k's” being identical to or different from each other;
each R¹¹ represents a carbonyl group or a hydrocarbylene group having 1 to 40 carbon atoms and optionally containing a heteroatom, the R¹¹'s being identical to or different from each other;
each R¹² represents a carbonyl group or a hydrocarbylene group having 1 to 10 carbon atoms and optionally containing a heteroatom, the R¹²'s being identical to or different from each other; and
each R¹³ represents a halogen atom or a hydrocarbyl group having 1 to 40 carbon atoms and optionally containing a heteroatom, the R¹³'s being identical to or different from each other.

In the general formula (1), “m” represents an integer of 0 to 2, “n” represents an integer of 1 to 40, and “k” represents an integer of 0 to 2m+4. “m” is preferably 0 or 1, particularly preferably 0. “n” is preferably 1 to 10, more preferably 1 to 4, further preferably 1 or 2, and most preferably 1. “k” is preferably 0 to 8, more preferably 0 to 6, further preferably 0 to 4, even more preferably 0 to 2, and most preferably 0 or 1. The “m's” and the “k's” may be identical to or different from each other.

In the general formula (1), R¹¹ represents a carbonyl group or a hydrocarbylene group having 1 to 40 carbon atoms and optionally containing a heteroatom. The hydrocarbylene group having 1 to 40 carbon atoms may be saturated or unsaturated, and may be linear, branched, or cyclic. Specific examples thereof include: alkylene groups having 1 to 40 carbon atoms, such as a methanediyl group, an ethane-1,1-diyl group, an ethane-1,2-diyl group, a propane-1,1-diyl group, a propane-1,2-diyl group, a propane-1,3-diyl group, a propane-2,2-diyl group, a butane-2,3-diyl group, a butane-1,4-diyl group, a 2-methylpropane-1,2-diyl group, a pentane-1,5-diyl group, a hexane-1,6-diyl group, a heptane-1,7-diyl group, an octane-1,8-diyl group, a nonane-1,9-diyl group, and a decane-1,10-diyl group; cyclic saturated hydrocarbylene groups having 3 to 40 carbon atoms, such as a cyclopentanediyl group, a cyclohexanediyl group, a norbornanediyl group, an adamantanediyl group, and a tricyclo[5.2.1.0^{2,6}]decanediyl group; alkenylene groups having 2 to 40 carbon atoms, such as a vinylene group and a propynylene group; arylene groups having 6 to 40 carbon atoms, such as a phenylene group, a methylphenylene group, an ethylphenylene group, an n-propylphenylene group, an isopropylphenylene group, an n-butylphenylene group, and a naphthylene group; and groups which are combinations of these groups. Furthermore, part or all of the hydrogen atoms of the hydrocarbylene group may be substituted with a group containing a heteroatom, such as an oxygen atom, a sulfur atom, a nitrogen atom, or a halogen atom, and part of the —CH₂— of the hydrocarbylene group may be substituted with a group containing a heteroatom such as an oxygen atom, a sulfur atom, or a nitrogen atom. The resulting hydrocarbylene group may contain a hydroxy group, a cyano group, a halogenated alkyl group, a halogen atom, a carbonyl group, an ether bond, a thioether bond, an ester bond, a sulfonic acid ester bond, a carbonate bond, a carbamate bond, a lactone ring, a sultone ring, a carboxylic anhydride (—C(═O)—O—C(═O)—), etc. As R¹¹, a carbonyl group, a hydrocarbylene group having 1 to 10 carbon atoms, or a fluorinated hydrocarbylene group having 1 to 10 carbon atoms is preferable. The R¹¹'s may be identical to or different from each other.

In the general formula (1), R¹² represents a carbonyl group or a hydrocarbylene group having 1 to 10 carbon atoms and optionally containing a heteroatom. The hydrocarbylene group having 1 to 10 carbon atoms may be saturated or unsaturated, and may be linear, branched, or cyclic. Specific examples thereof include: alkylene groups having 1 to 10 carbon atoms, such as a methanediyl group, an ethane-1,1-diyl group, an ethane-1,2-diyl group, a propane-1,1-diyl group, a propane-1,2-diyl group, a propane-1,3-diyl group, a propane-2,2-diyl group, a butane-2,3-diyl group, a butane-1,4-diyl group, a 2-methylpropane-1,2-diyl group, a pentane-1,5-diyl group, a hexane-1,6-diyl group, a heptane-1,7-diyl group, an octane-1,8-diyl group, a nonane-1,9-diyl group, and a decane-1,10-diyl group; cyclic saturated hydrocarbylene groups having 3 to 10 carbon atoms, such as a cyclopentanediyl group, a cyclohexanediyl group, a norbornanediyl group, an adamantanediyl group, and a tricyclo[5.2.1.0^{2,6}]decanediyl group; alkenylene groups having 2 to 10 carbon atoms, such as a vinylene group and a propynylene group; arylene groups having 6 to 10 carbon atoms, such as a phenylene group, a methylphenylene group, an ethylphenylene group, an n-propylphenylene group, an isopropylphenylene group, an n-butylphenylene group, and a naphthylene group; and groups which are combinations of these groups. Furthermore, part or all of the hydrogen atoms of the hydrocarbylene group may be substituted with a group containing a heteroatom, such as an oxygen atom, a sulfur atom, a nitrogen atom, or a halogen atom, and part of the —CH₂— of the hydrocarbylene group may be substituted with a group containing a heteroatom such as an oxygen atom, a sulfur atom, or a nitrogen atom. The resulting hydrocarbylene group may contain a hydroxy group, a cyano group, a halogenated alkyl group, a halogen atom, a carbonyl group, an ether bond, a thioether bond, an ester bond, a sulfonic acid ester bond, a carbonate bond, a carbamate bond, a lactone ring, a sultone ring, a carboxylic anhydride (—C(═O)—O—C(═O)—), etc. As R¹², a carbonyl group, a hydrocarbylene group having 1 to 4 carbon atoms, or a fluorinated hydrocarbylene group having 1 to 4 carbon atoms is preferable. The R¹²'s may be identical to or different from each other.

In the general formula (1), R¹³ represents a halogen atom or a hydrocarbyl group having 1 to 40 carbon atoms and optionally containing a heteroatom. Specific examples of the halogen atom include a fluorine atom, a chlorine atom, a bromine atom, and an iodine atom. The hydrocarbyl group having 1 to 40 carbon atoms may be saturated or unsaturated, and may be linear, branched, or cyclic. Specific examples thereof include: alkyl groups having 1 to 40 carbon atoms, such as a methyl group, an ethyl group, an n-propyl group, an isopropyl group, an n-butyl group, an isobutyl group, a sec-butyl group, a tert-butyl group, an n-pentyl group, a tert-pentyl group, an n-hexyl group, an n-octyl group, a 2-ethylhexyl group, an n-nonyl group, and an n-decyl group; cyclic saturated hydrocarbyl groups having 3 to 40 carbon atoms, such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, a tricyclo[5.2.1.0^{2,6}]decanyl group, an adamantyl group, and an adamantylmethyl group; and aryl groups having 6 to 40 carbon atoms such as a phenyl group, a naphthyl group, and an anthracenyl group. Furthermore, part or all of the hydrogen atoms of the hydrocarbyl group may be substituted with a group containing a heteroatom, such as an oxygen atom, a sulfur atom, a nitrogen atom, or a halogen atom, and part of the —CH₂— of the hydrocarbyl group may be substituted with a group containing a heteroatom such as an oxygen atom, a sulfur atom, or a nitrogen atom. The resulting hydrocarbyl group may contain a hydroxy group, a cyano group, a halogen atom, a carbonyl group, an ether bond, a thioether bond, an ester bond, a sulfonic acid ester bond, a carbonate bond, a carbamate bond, a lactone ring, a sultone ring, a carboxylic anhydride (—C(═O)—O—C(═O)—), etc. The R¹³'s may be identical to or different from each other. Furthermore, multiple R¹³'s may be bonded to each other to form a ring together with the carbon atoms of the aromatic rings bonded thereto.

Specific examples of the hypervalent iodine compound represented by the general formula (1) include the following, but are not limited thereto. Note that, in the following formulae, Me represents a methyl group and Ph represents a phenyl group.

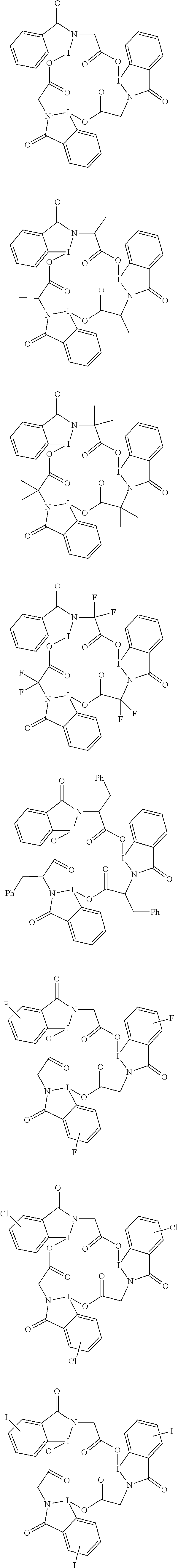

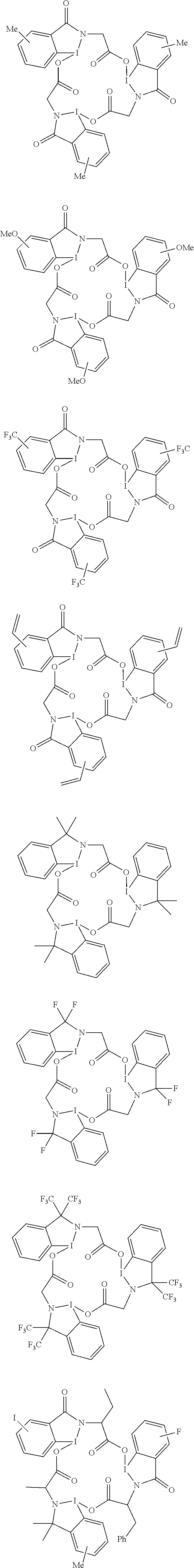

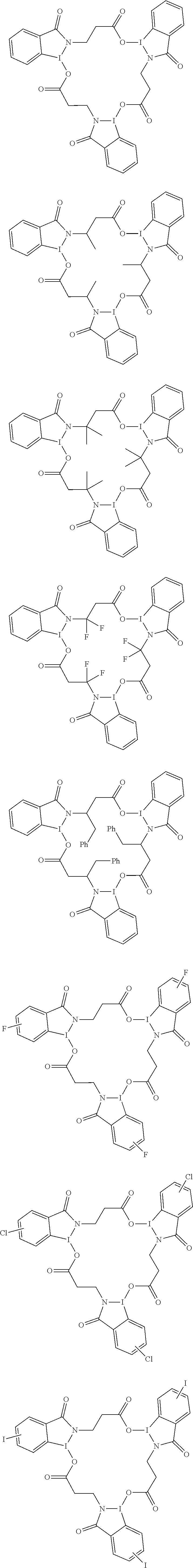

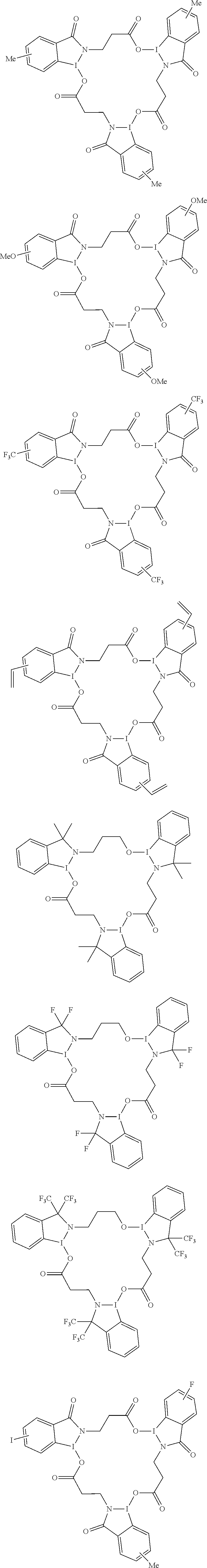

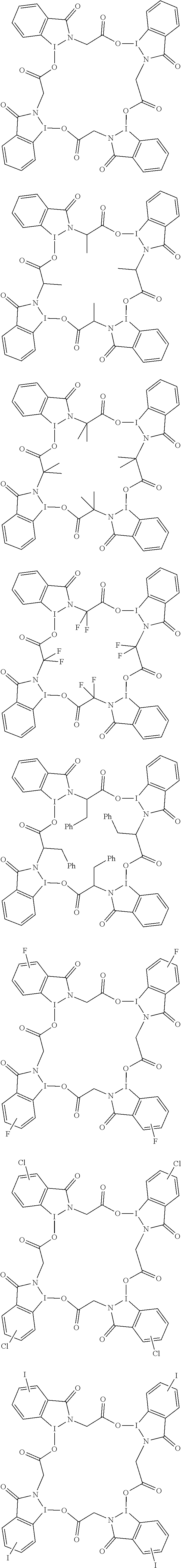

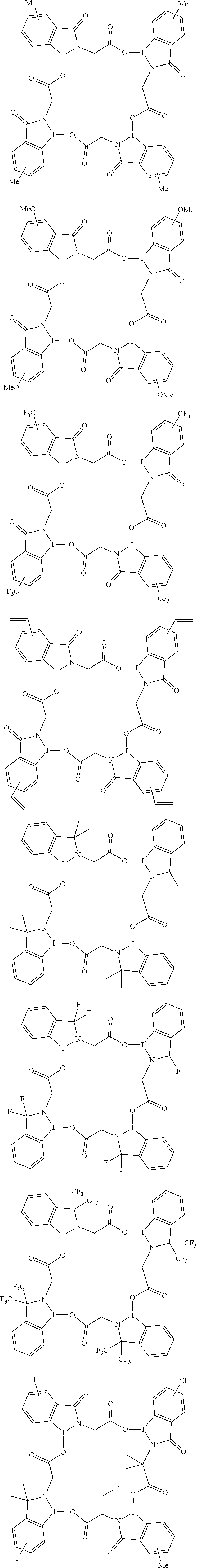

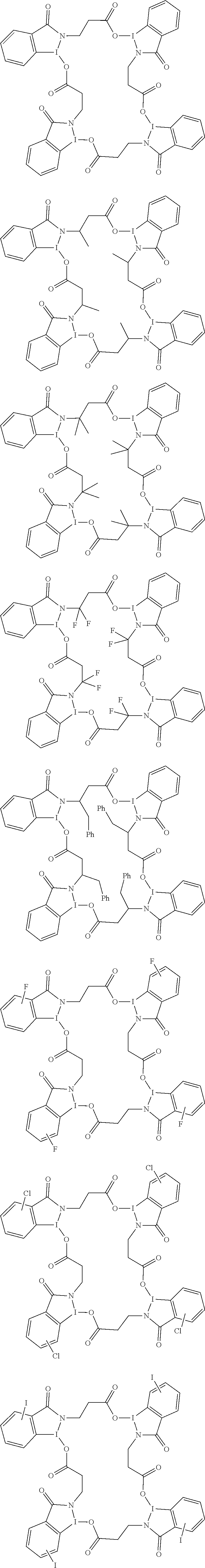

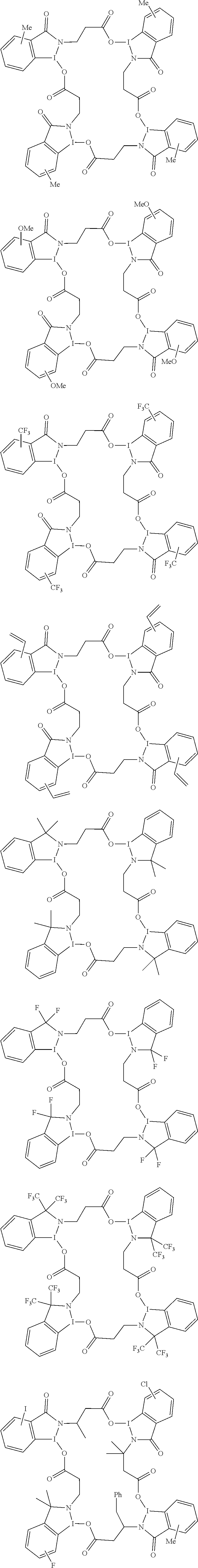

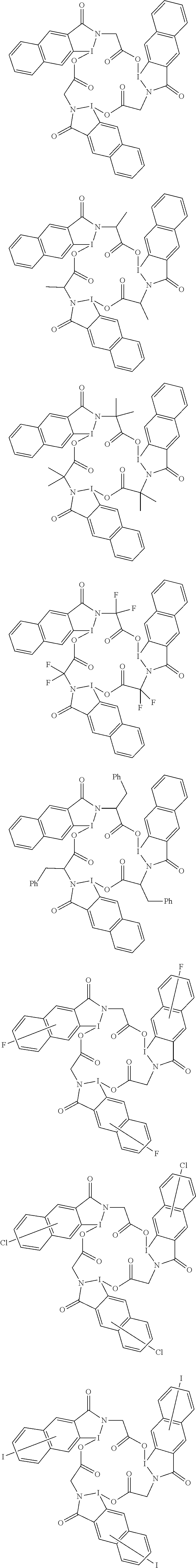

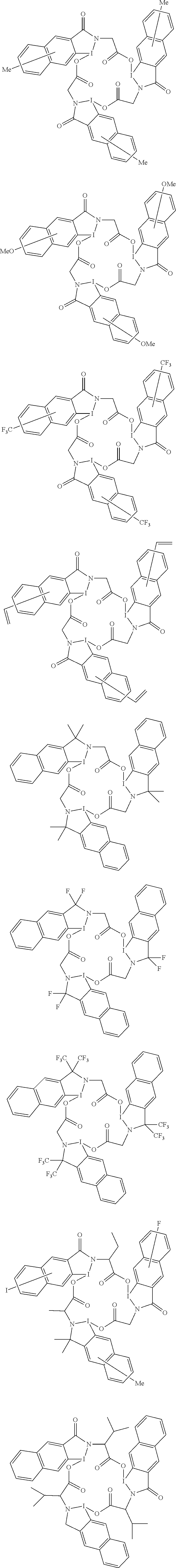

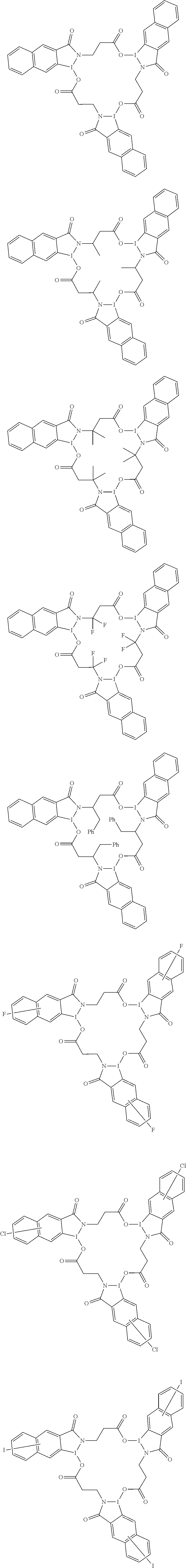

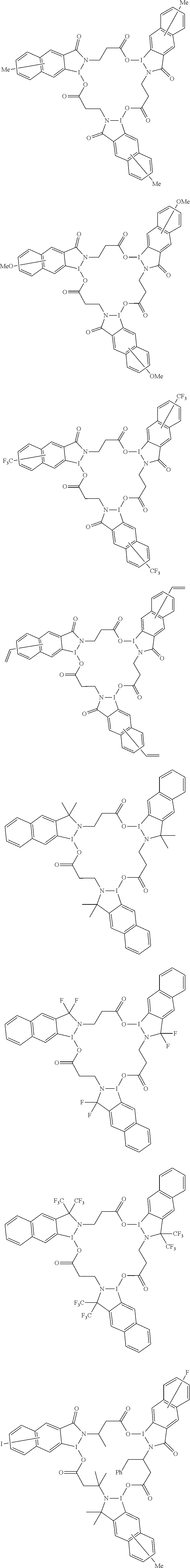

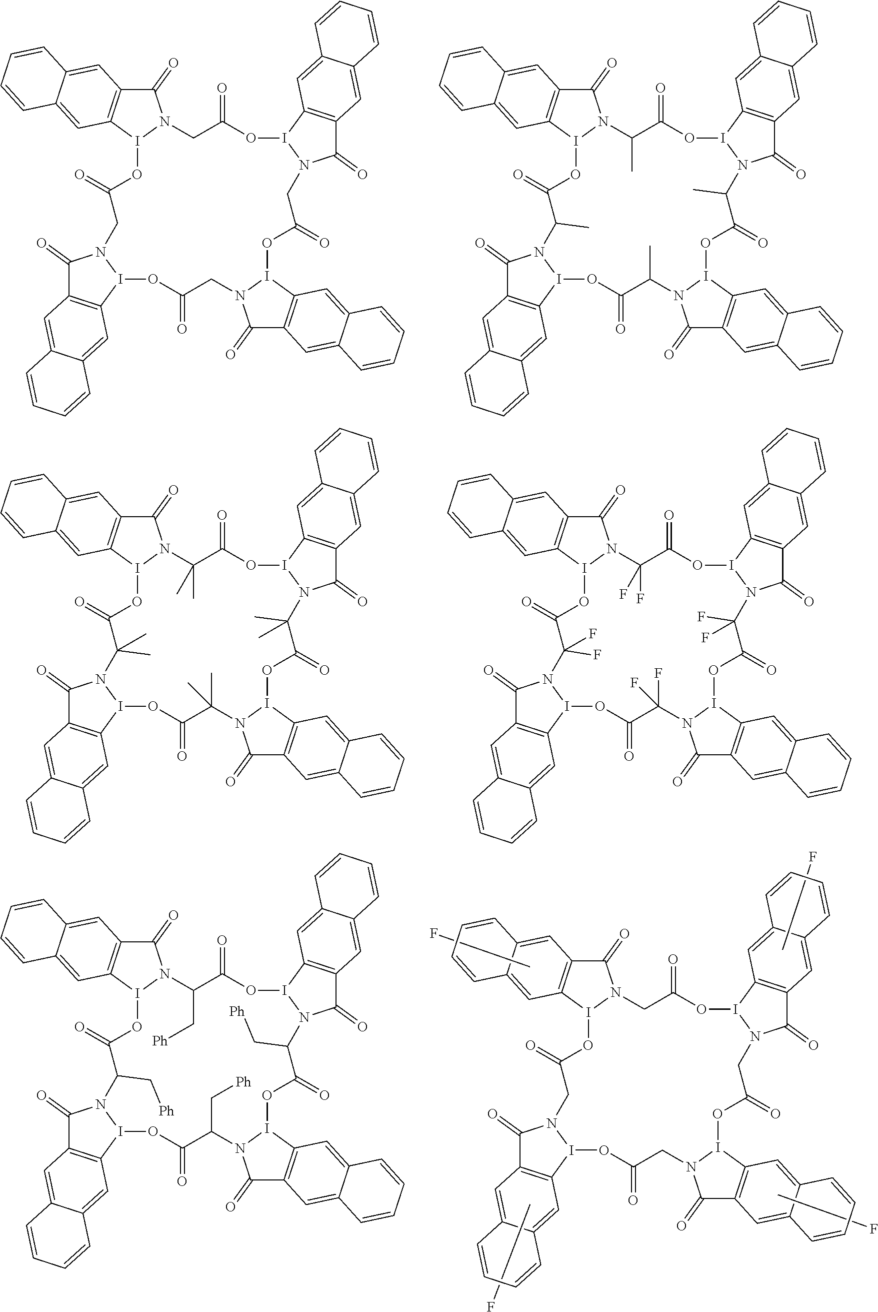

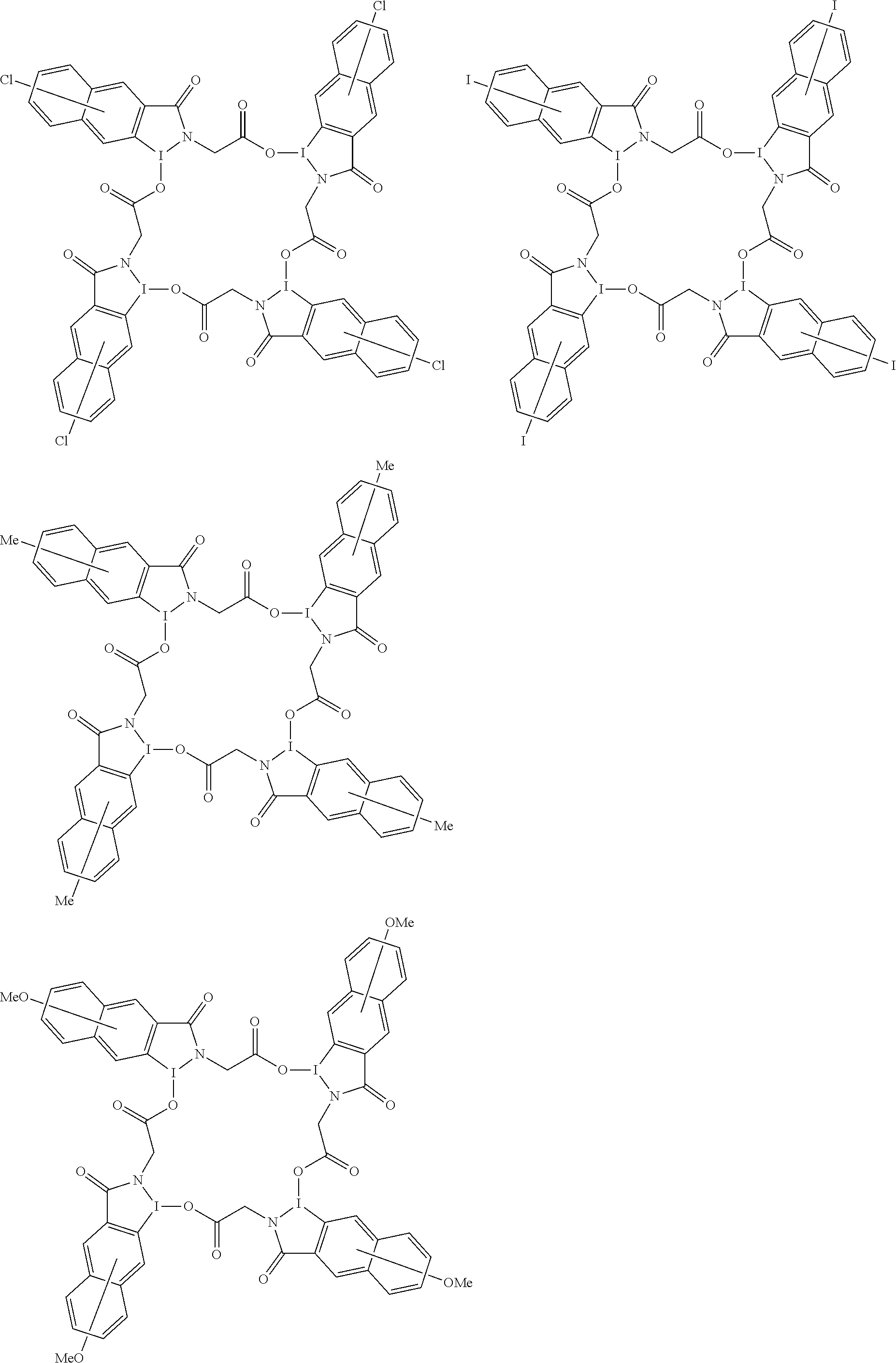

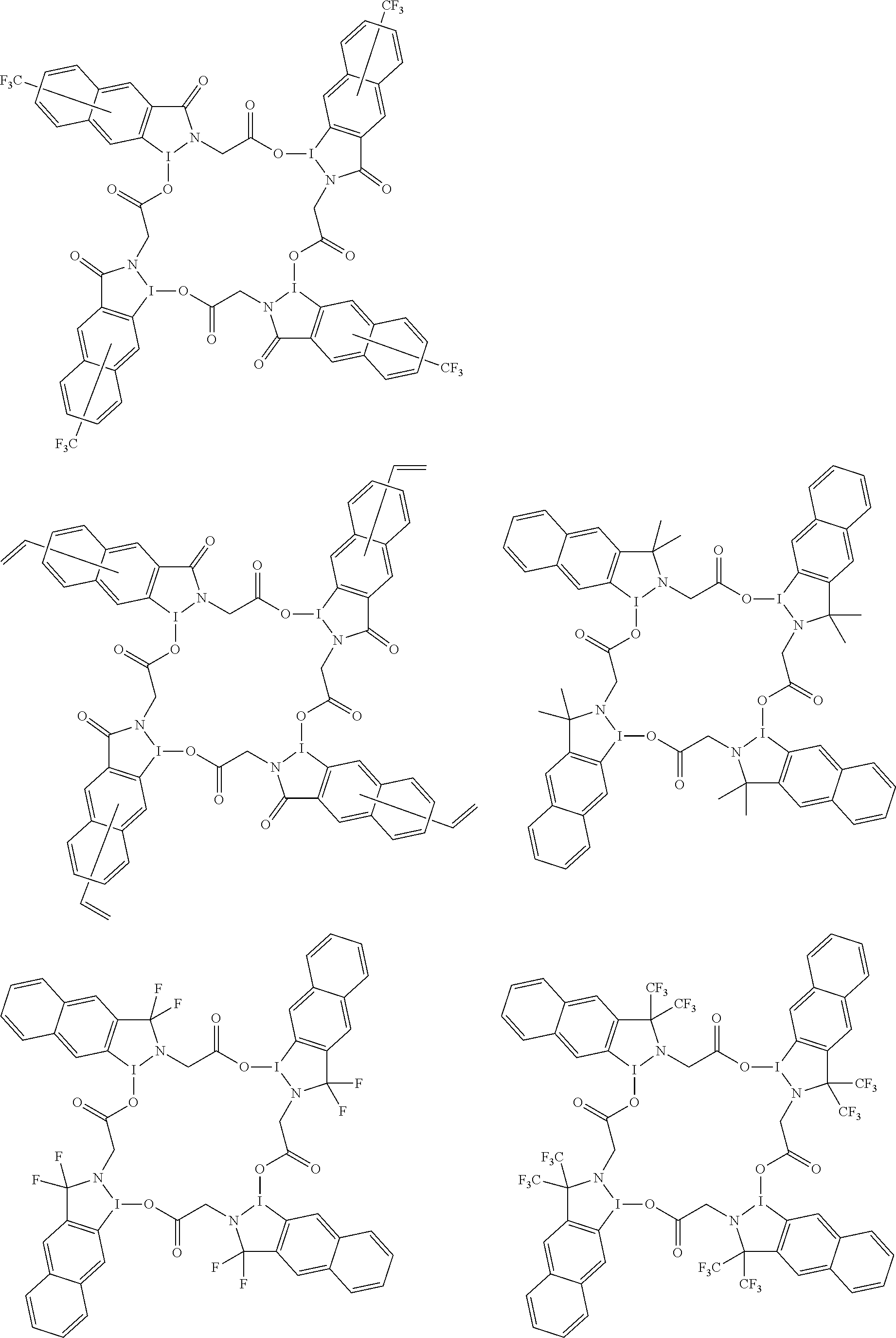

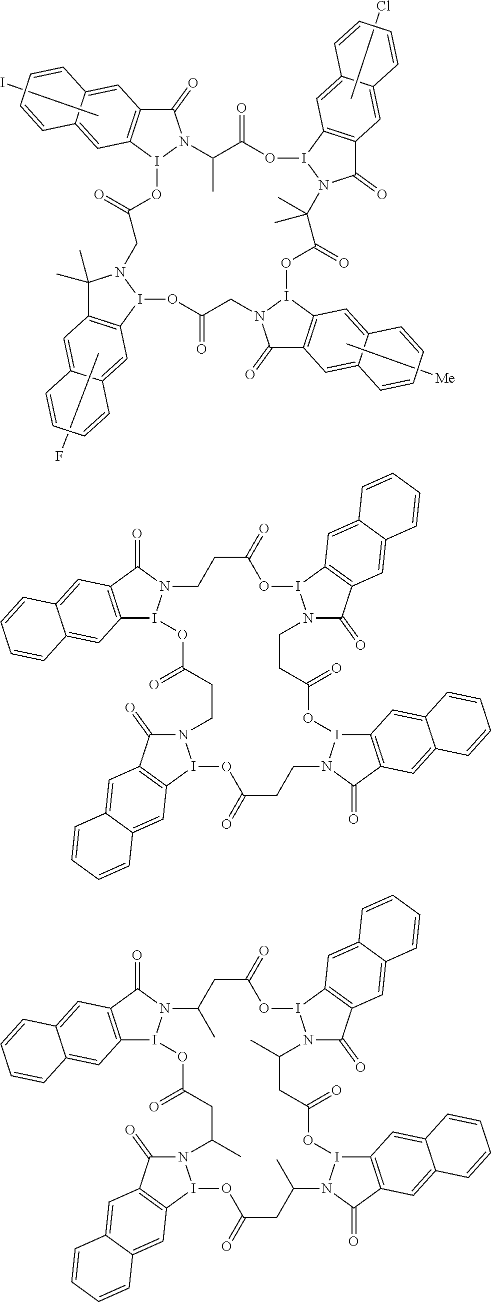

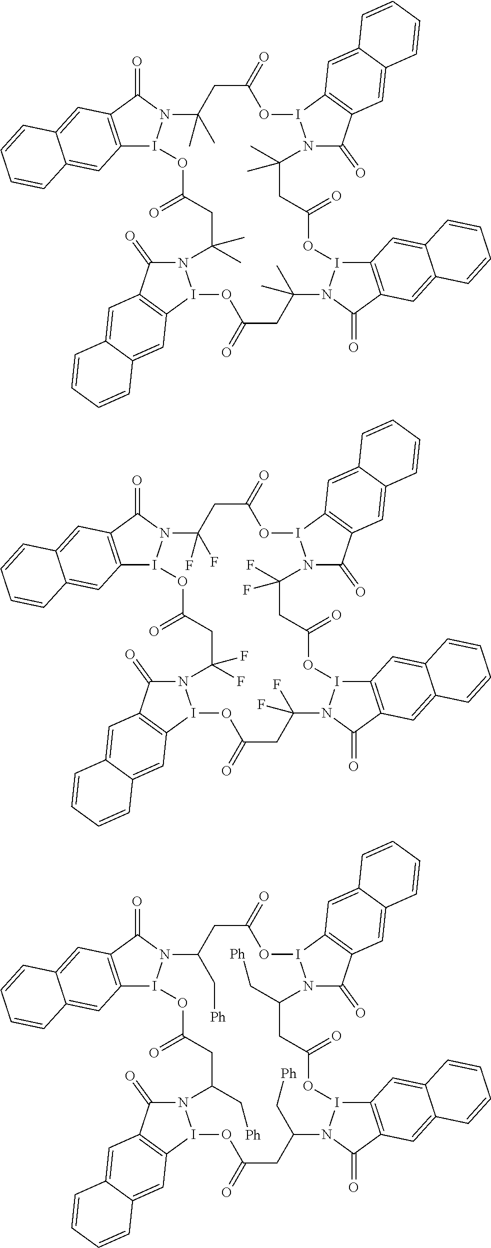

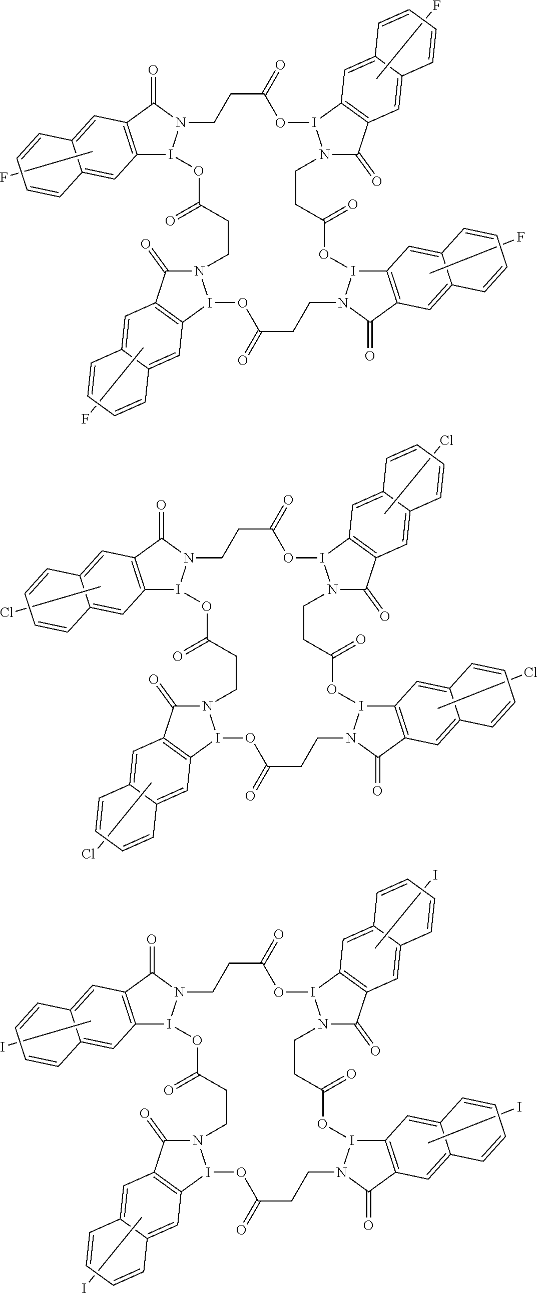

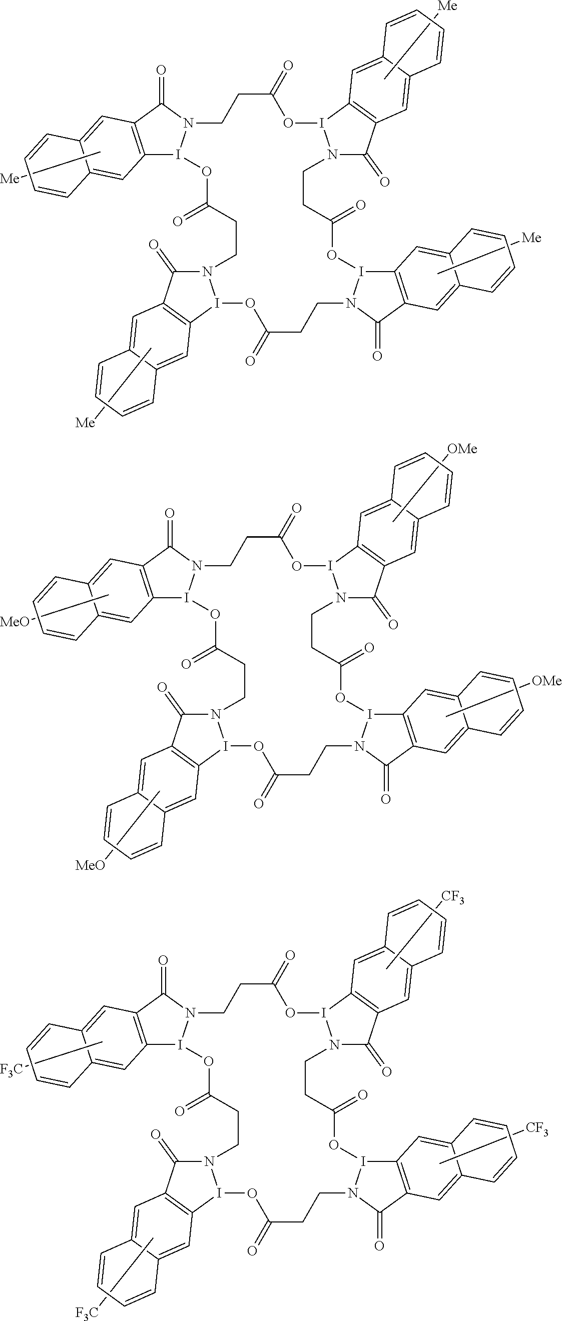

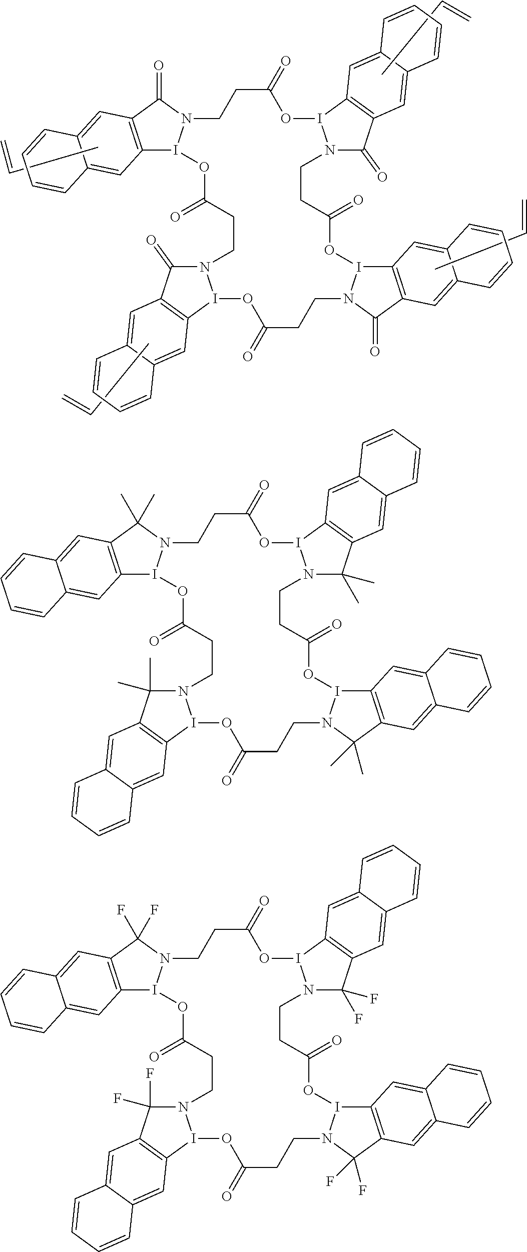

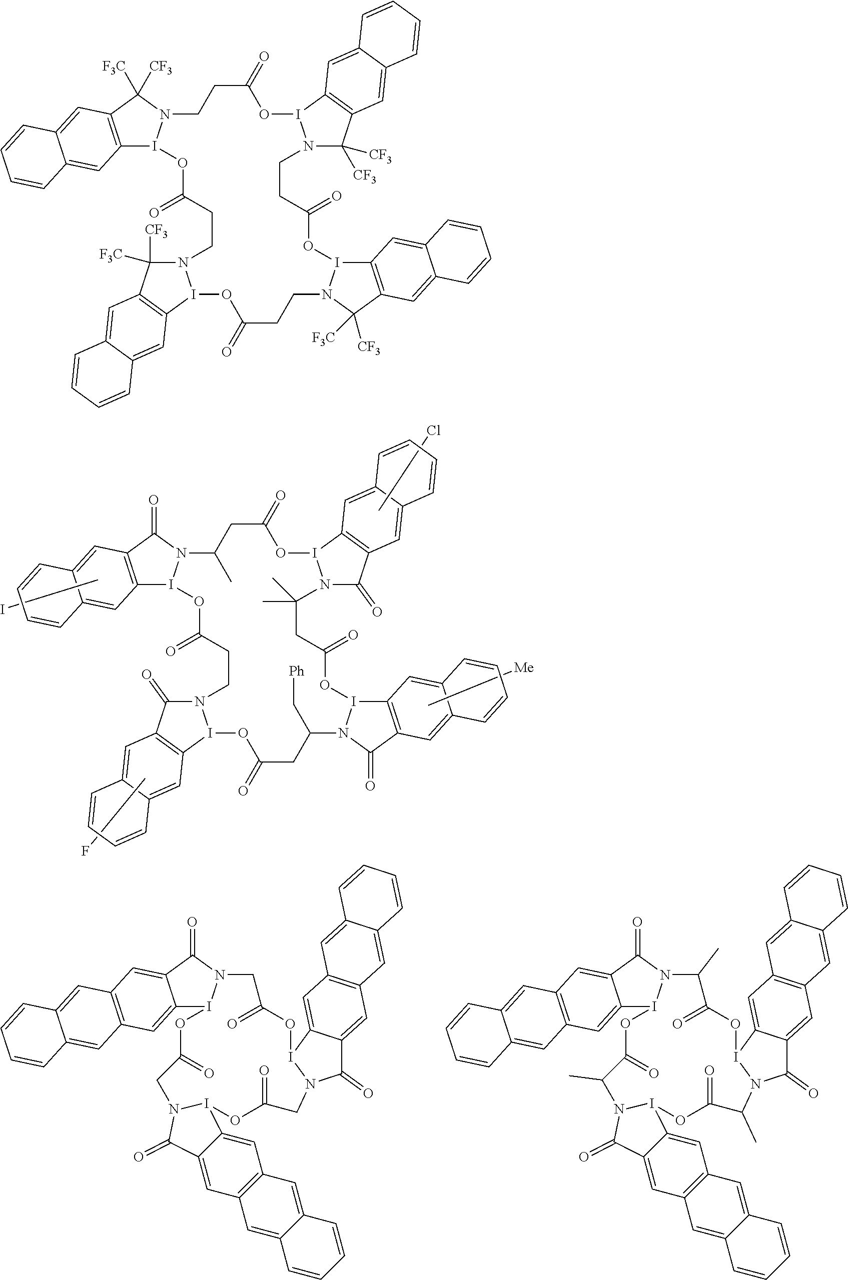

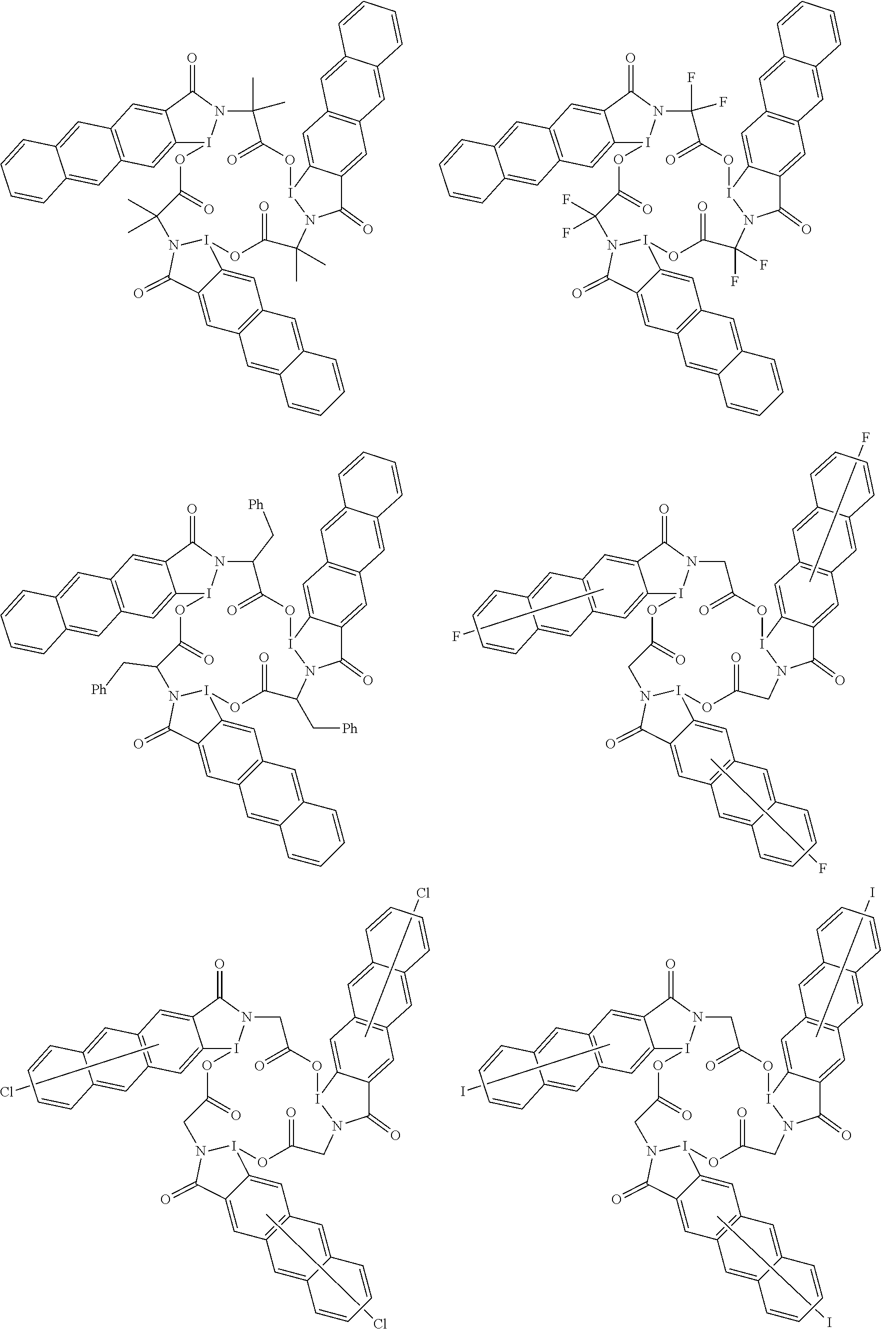

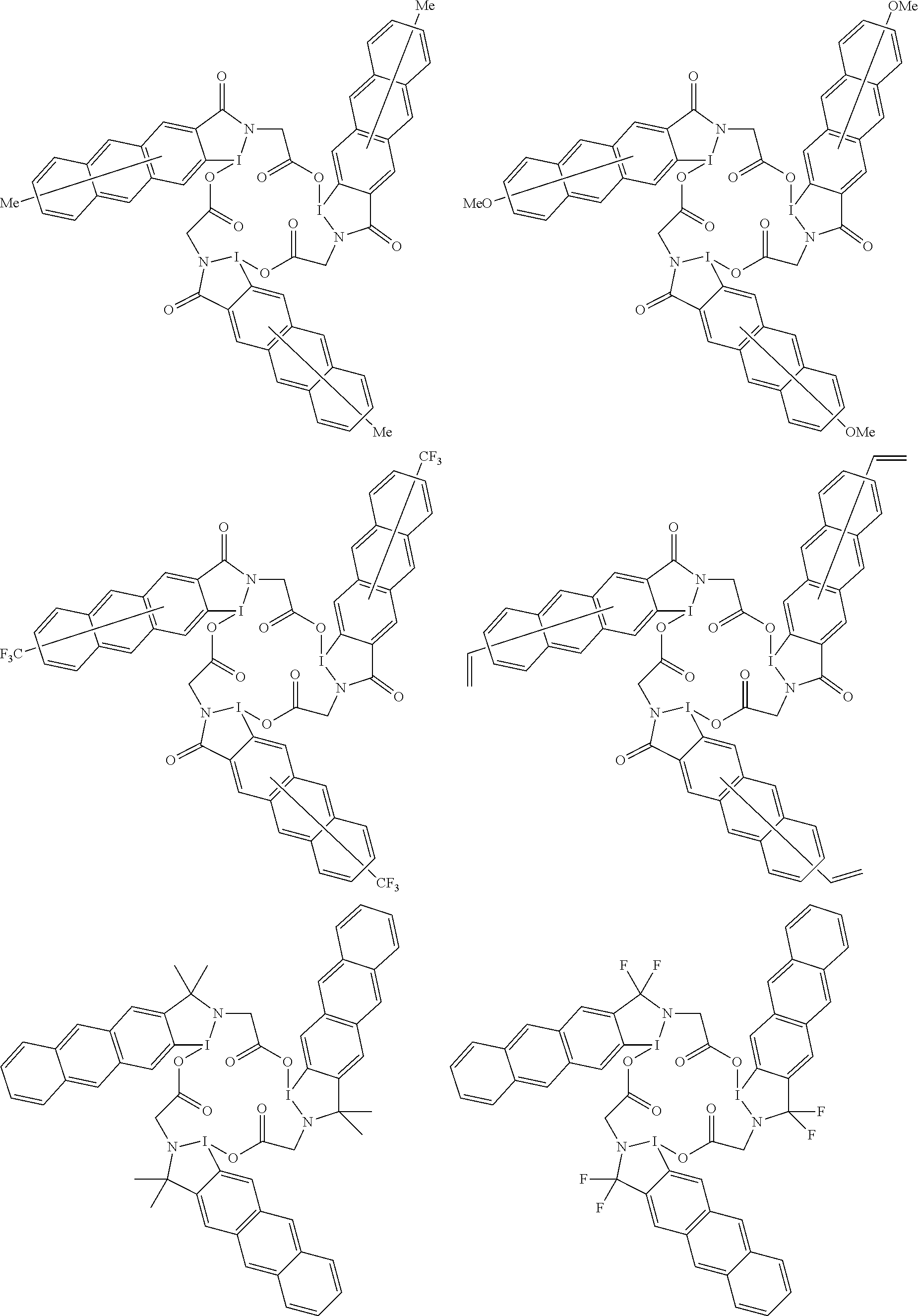

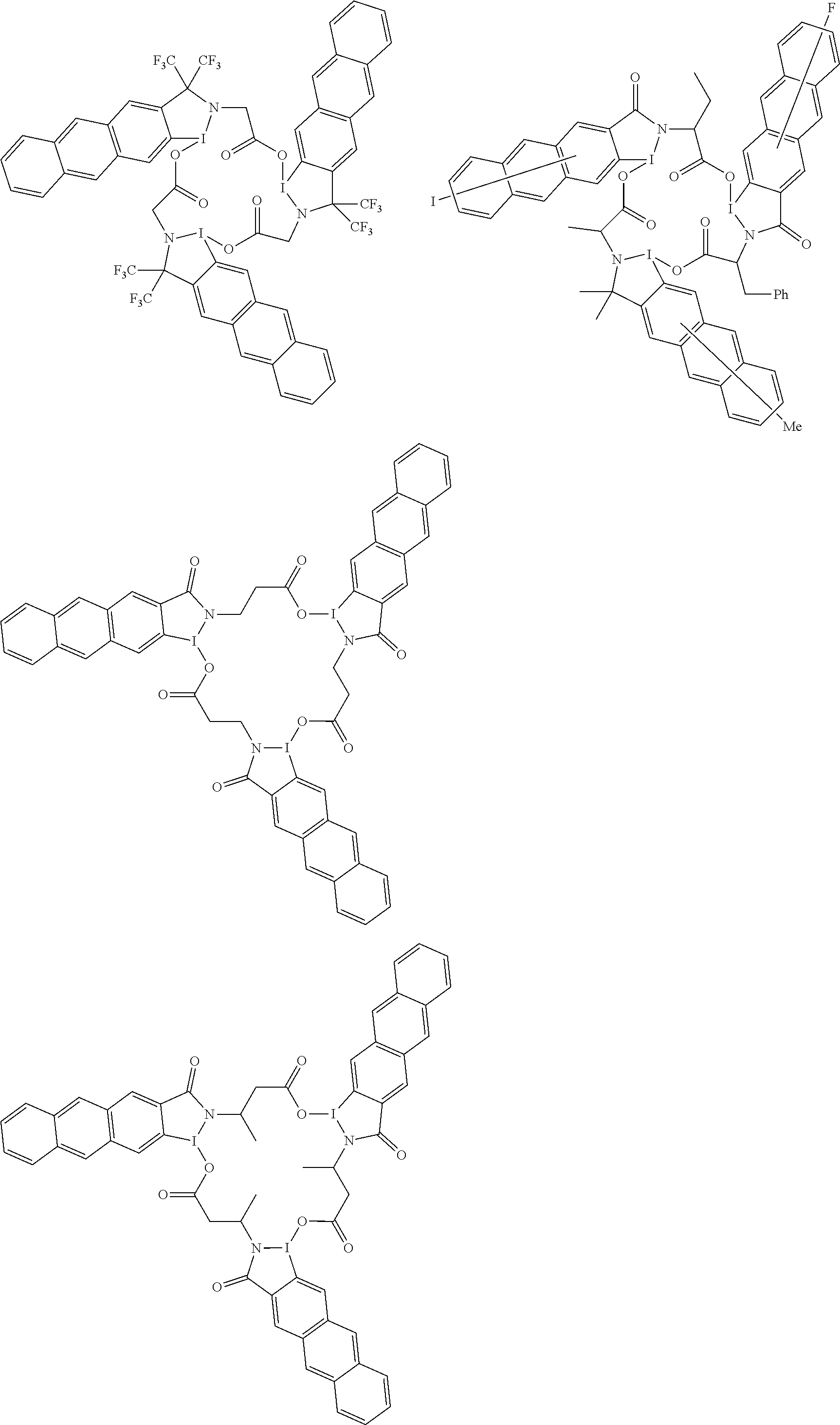

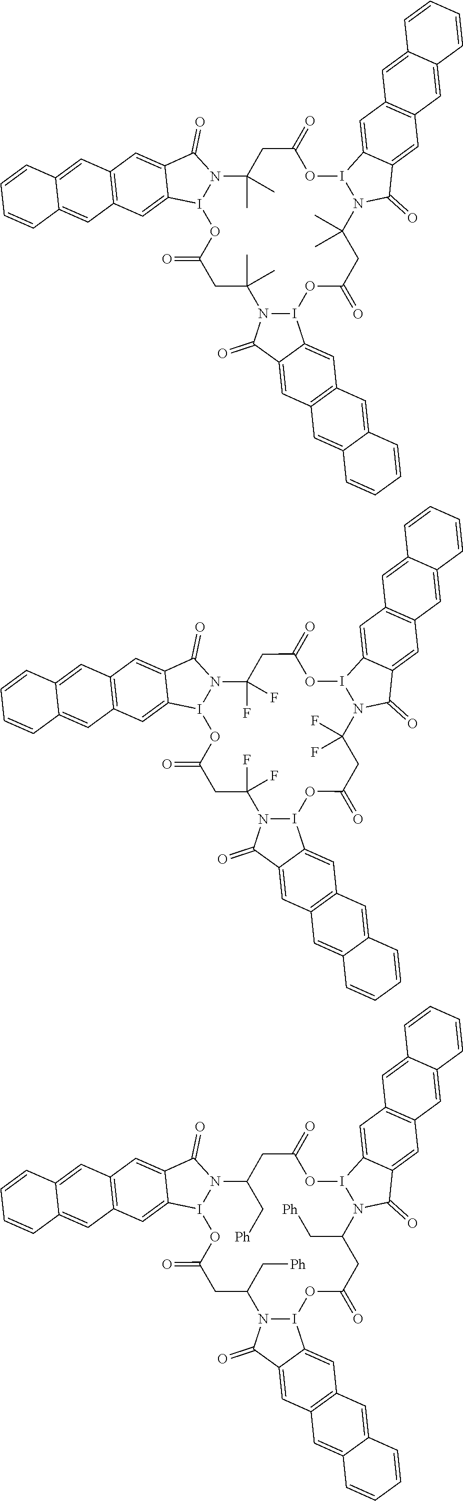

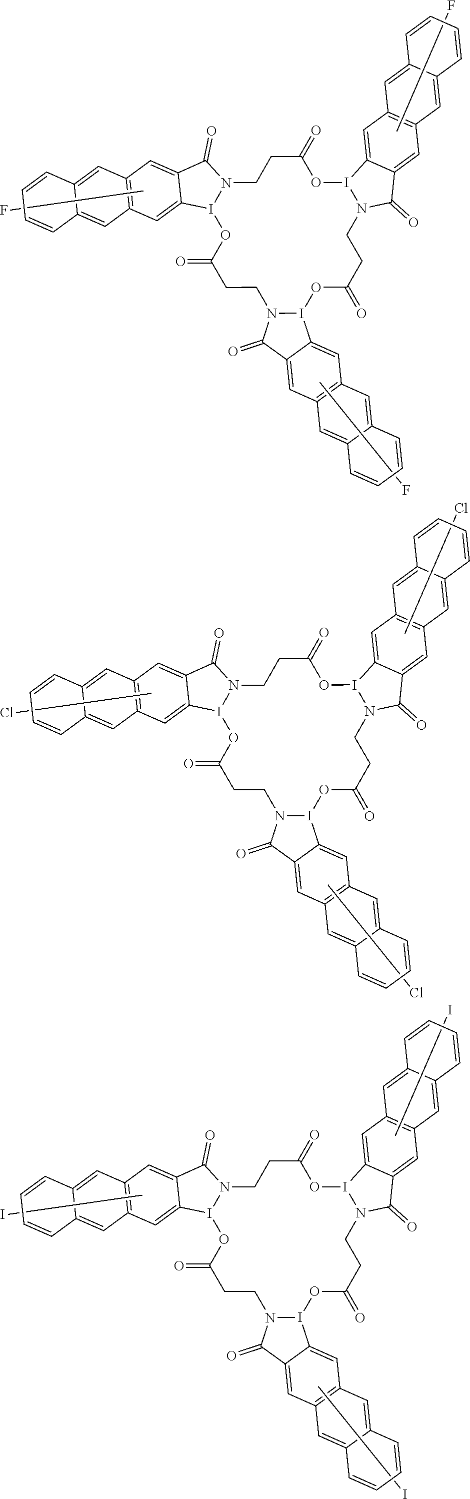

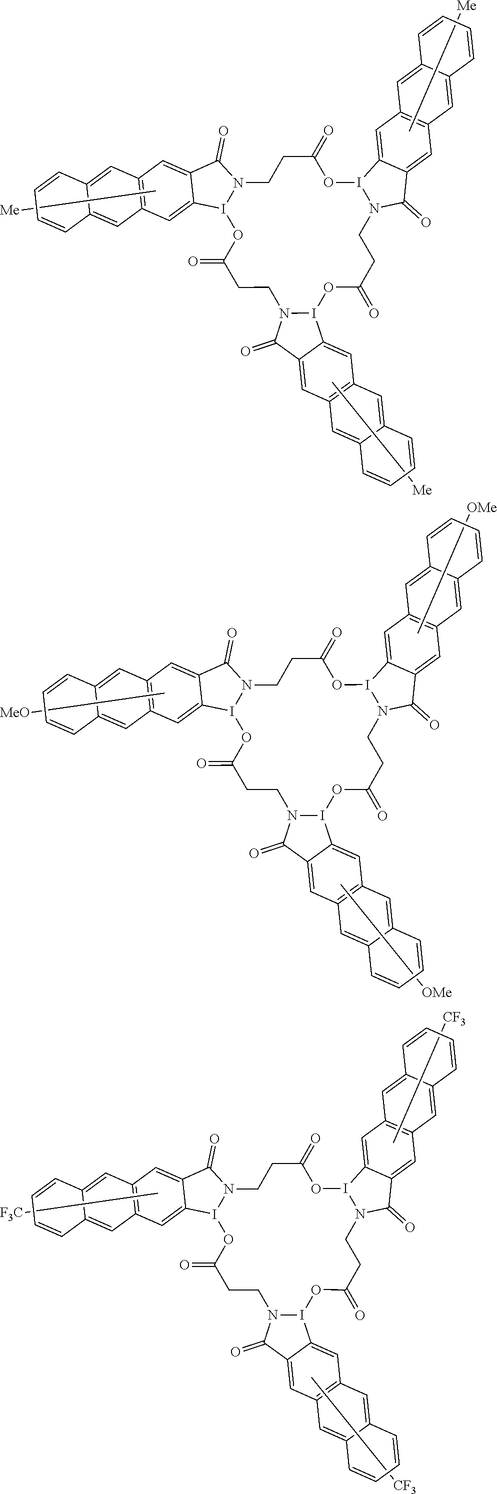

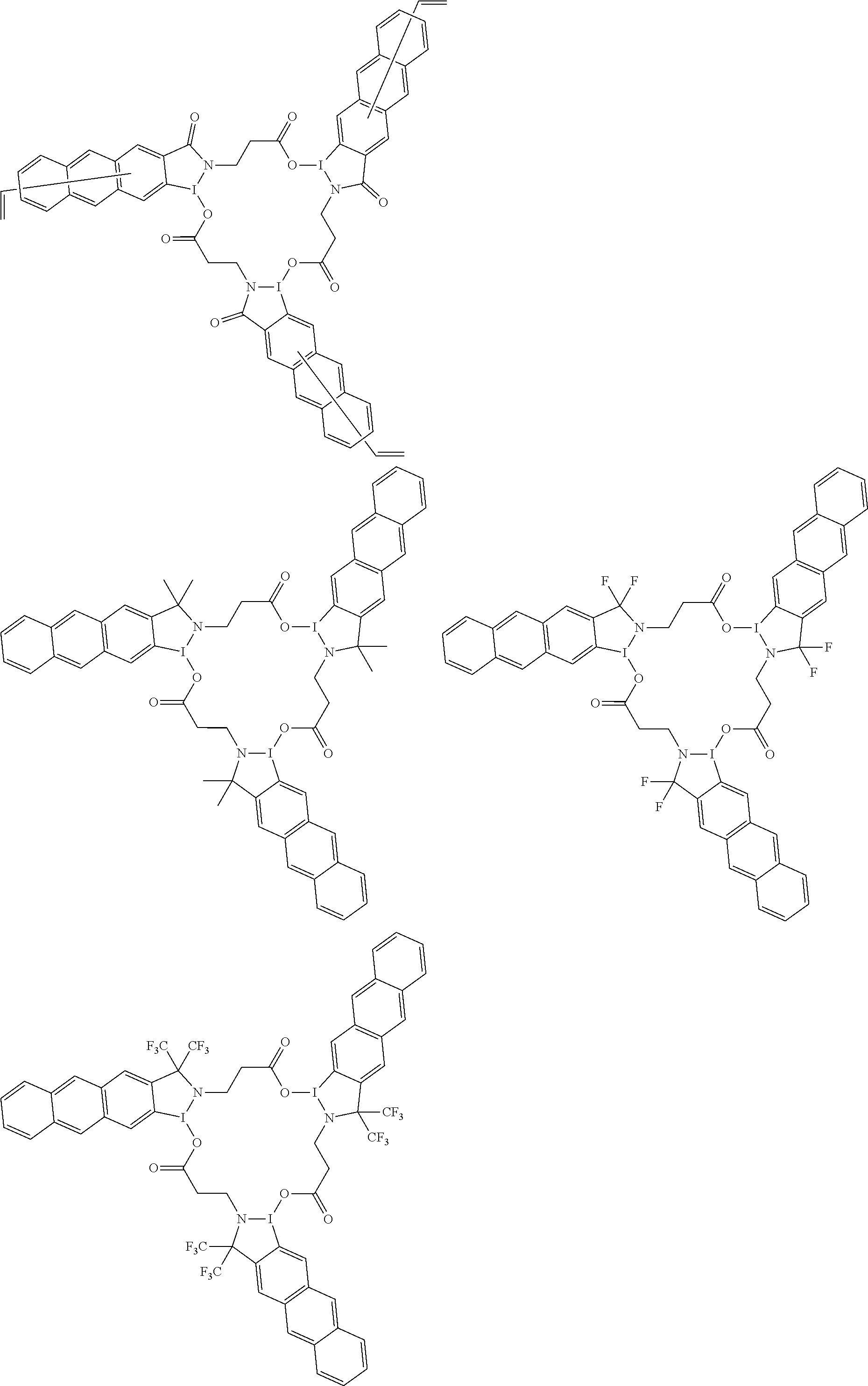

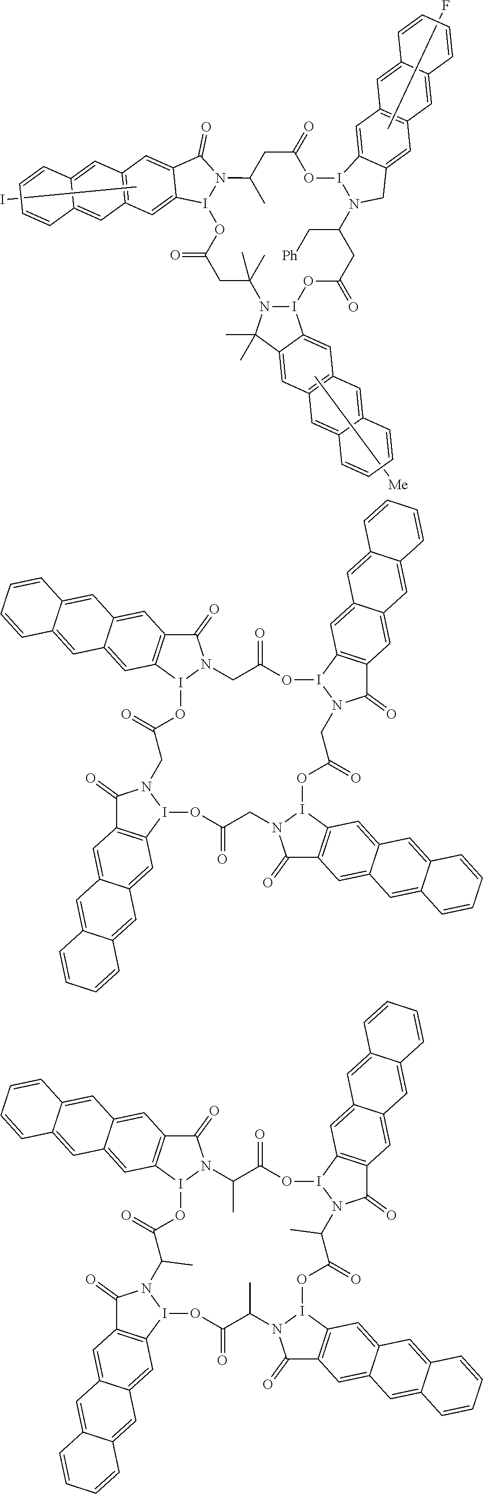

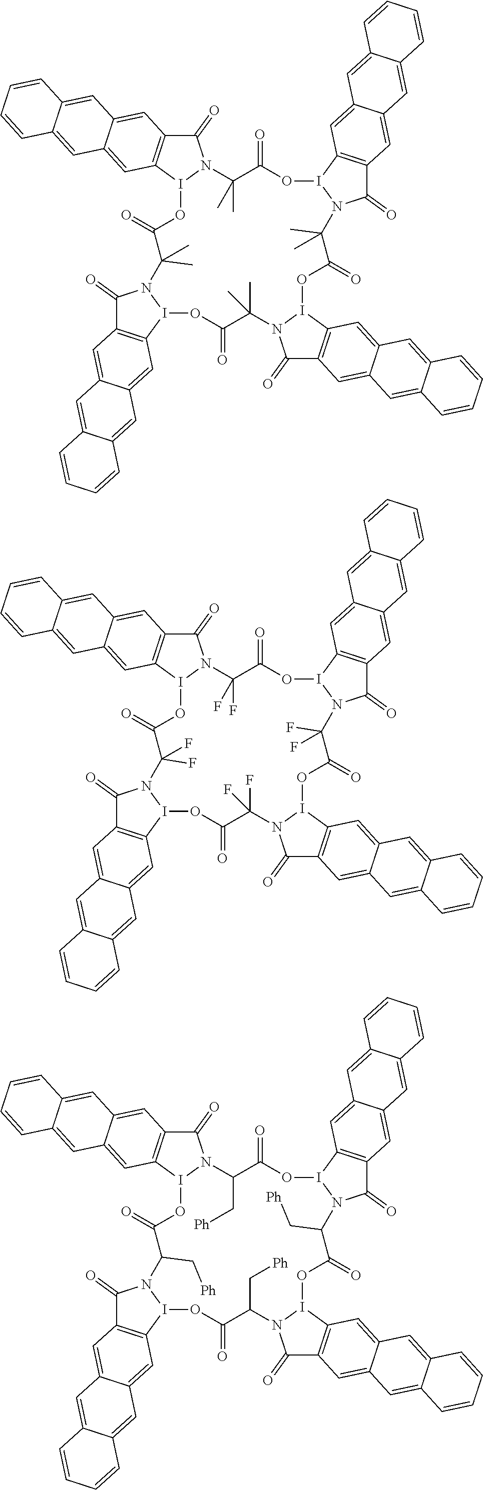

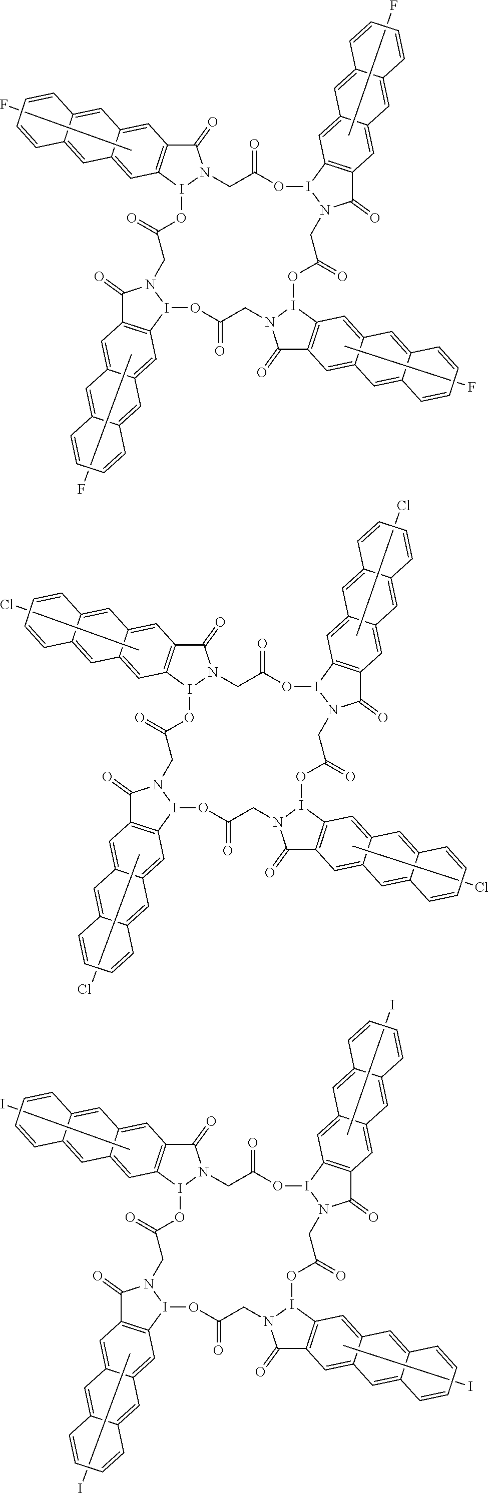

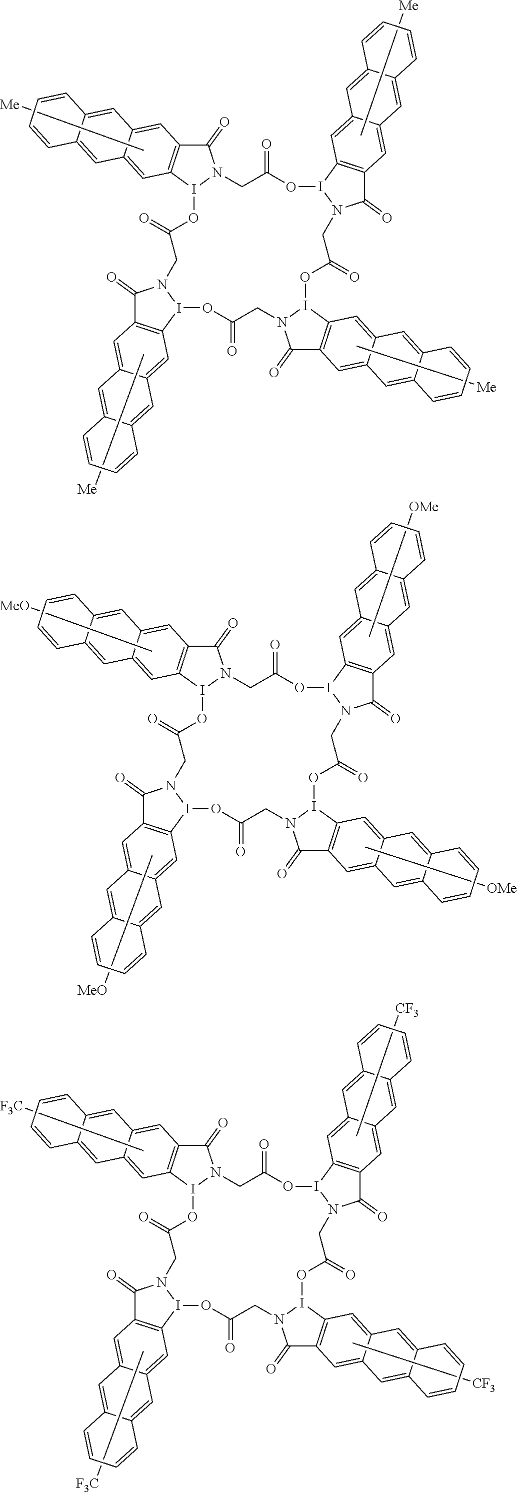

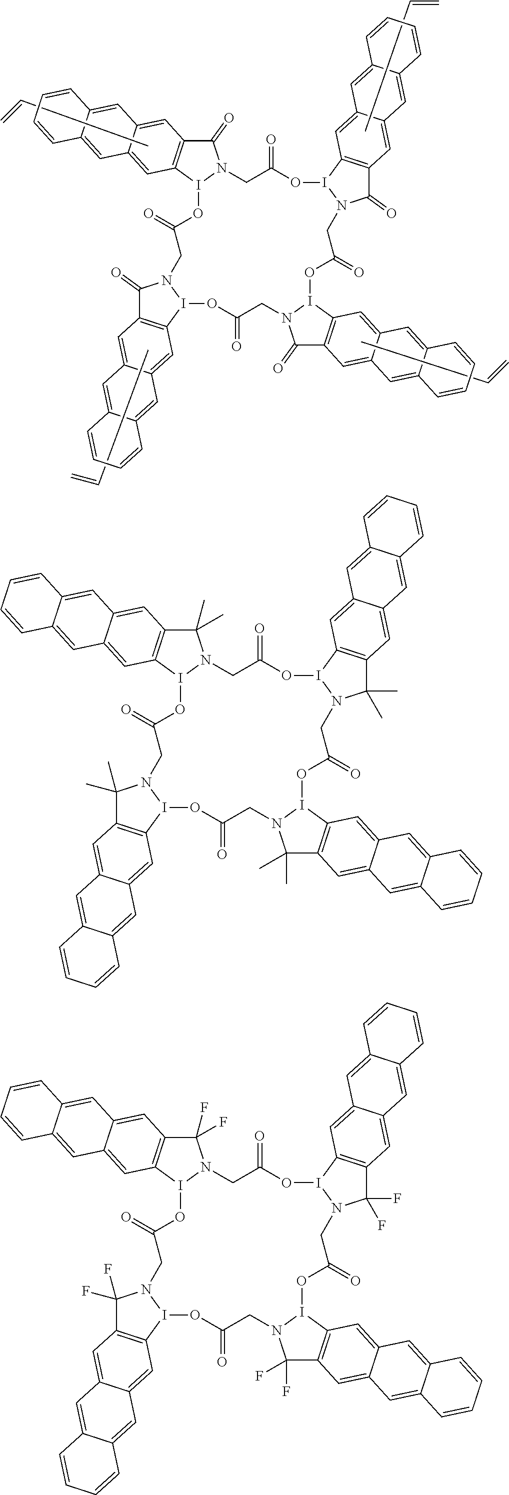

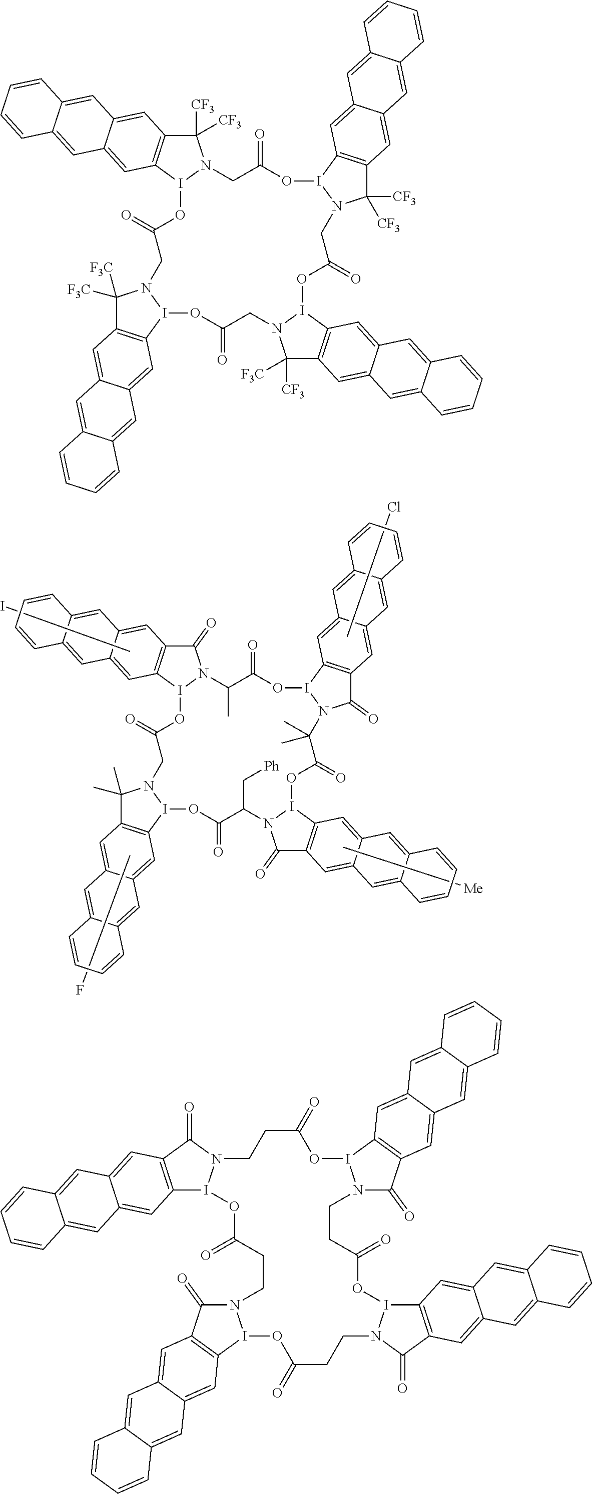

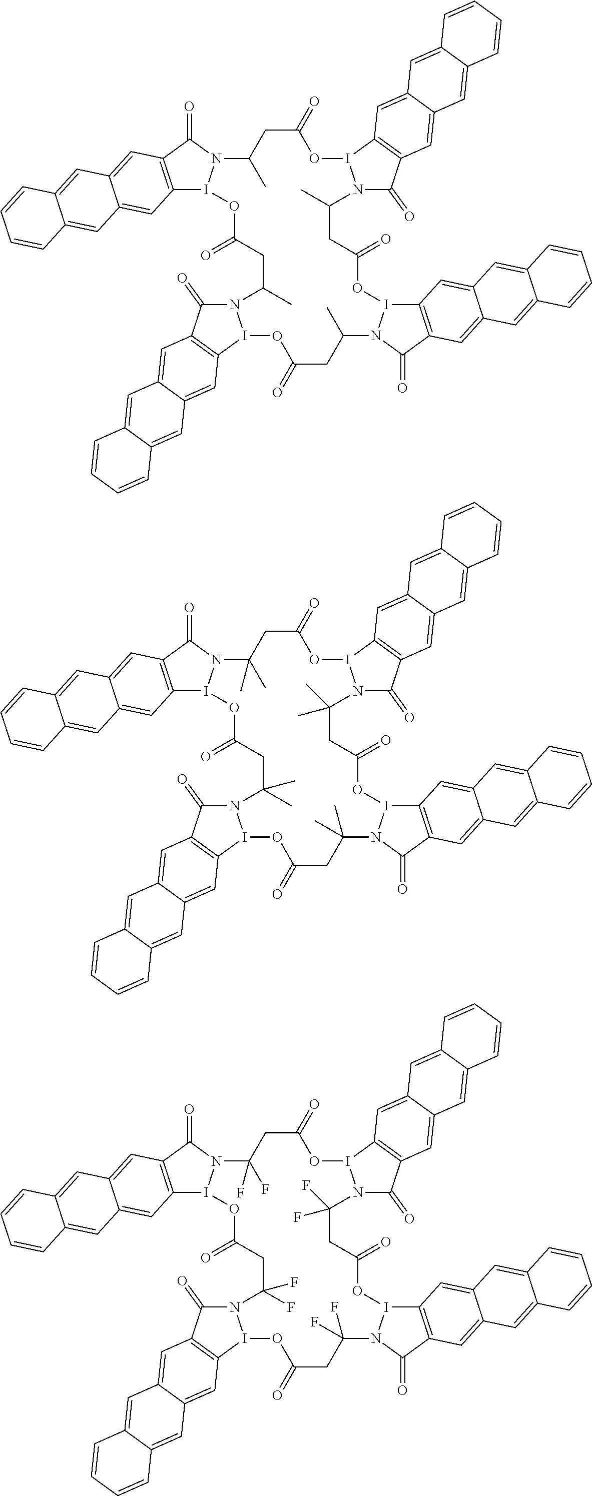

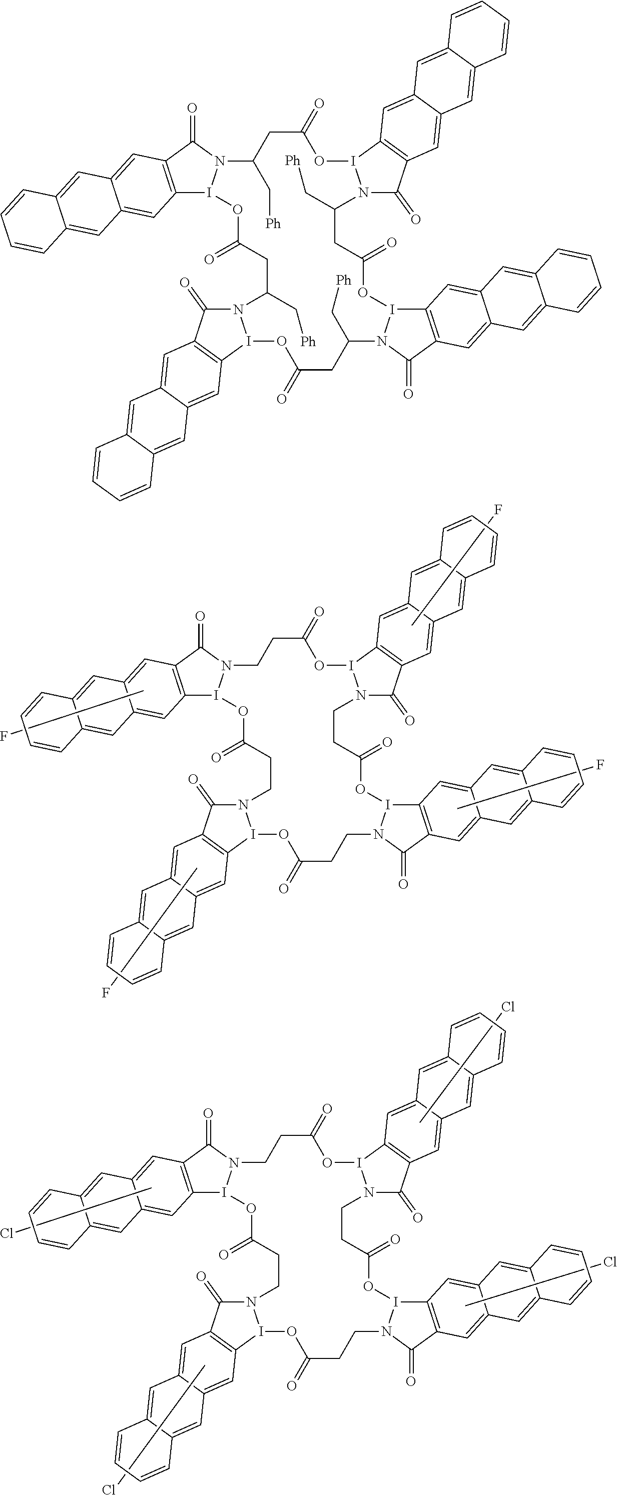

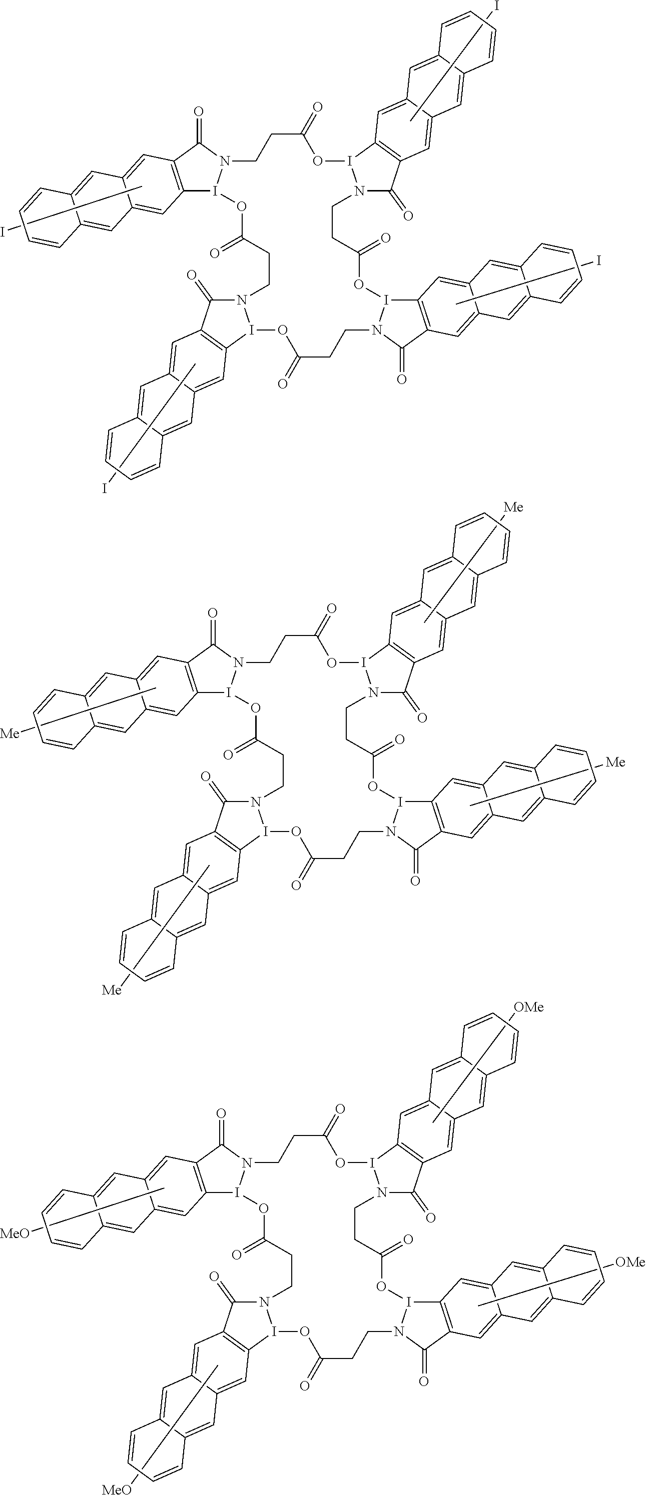

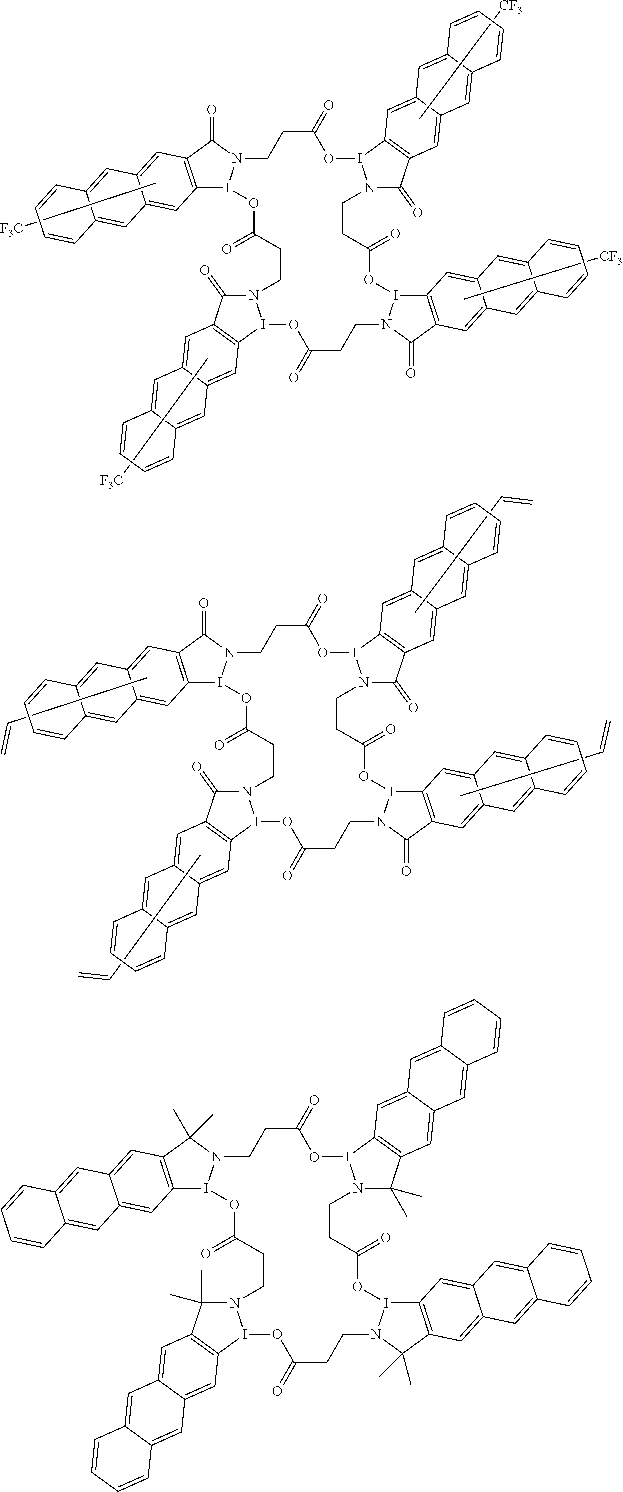

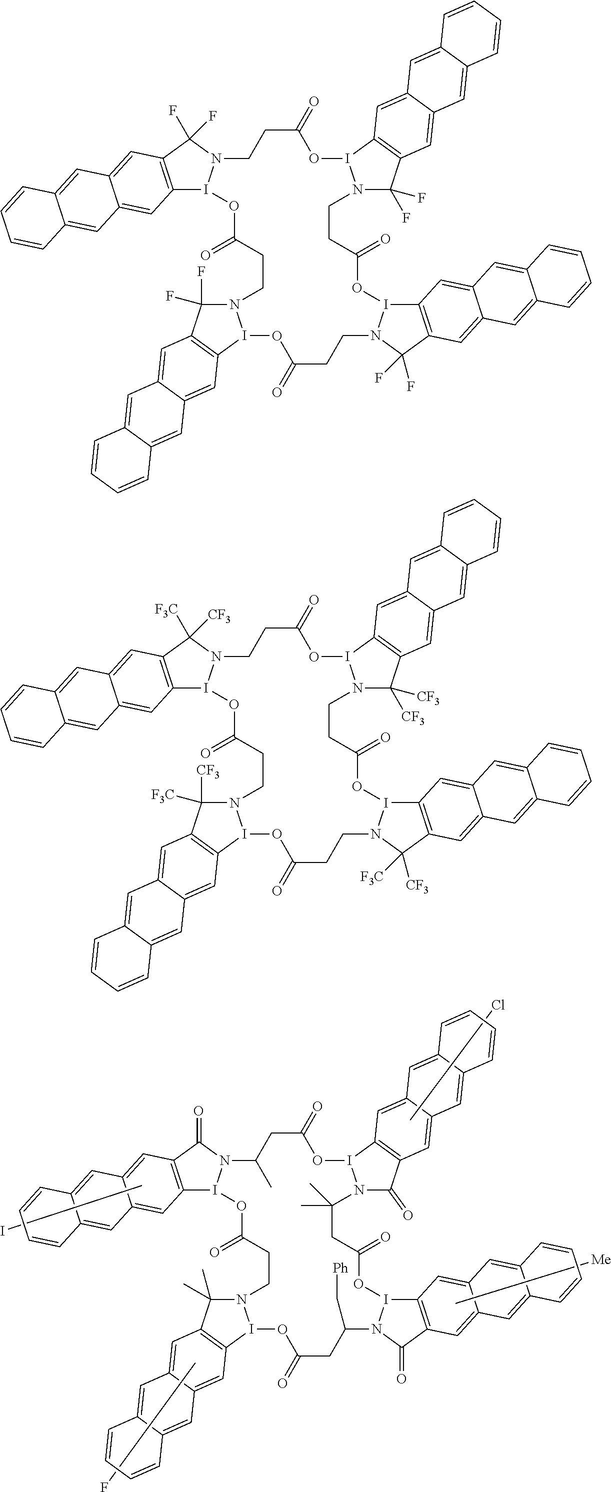

### [Method for Manufacturing Hypervalent Iodine Compound]

The hypervalent iodine compound used in the present invention can be obtained by a known method. For example, the compound can be obtained by preparing a 2-iodobenzamide derivative from a 2-iodobenzoic acid derivative and an amino acid derivative, and carrying out oxidative ring closure by using an oxidizing agent such as m-chloroperbenzoic acid. Regarding the synthesizing method, J. Mater. Chem. C, 2025, vol. 13, p. 842-848 etc. can be consulted, for example.

In the inventive resist composition, the hypervalent iodine compound represented by the general formula (1) is preferably contained in an amount of 0.01 to 50 mass % of the entire resist composition including the solvent, more preferably 0.01 to 10 mass %, and particularly preferably 1 to 10 mass %. One kind of the hypervalent iodine compound represented by the general formula (1) may be used, or two or more kinds thereof may be used in mixture.

### [Solvent]

The inventive resist composition contains a solvent. The solvent is not particularly limited as long as the solvent dissolves the hypervalent iodine compound represented by the general formula (1) and other components described later and allows the formation of a resist film by being contained in the resist composition. As such a solvent, organic solvents are preferable, and specific examples thereof include: ketones, such as cyclohexanone, methyl-2-n-pentyl ketone, and methyl isoamyl ketone; alcohols, such as 3-methoxybutanol, 3-methyl-3-methoxybutanol, 1-methoxy-2-propanol, 1-ethoxy-2-propanol, diacetone alcohol, 4-methyl-2-pentanol, and methyl 2-hydroxyisobutyrate; ethers, such as propylene glycol monomethyl ether, ethylene glycol monomethyl ether, propylene glycol monoethyl ether, ethylene glycol monoethyl ether, propylene glycol dimethyl ether, and diethylene glycol dimethyl ether; esters, such as propylene glycol monomethyl ether acetate, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; carboxylic acids, such as formic acid, acetic acid, and propionic acid; lactones, such as γ-butyrolactone; and mixed solvents thereof.

In the inventive resist composition, the amount of the solvent contained is preferably such an amount that the concentration of the solid contents in the resist composition is 0.1 to 20 mass %, more preferably 0.1 to 15 mass %, and further preferably 0.1 to 10 mass %. Note that, in the present invention, solid contents is a general term for the components other than the solvent out of all the components of the resist composition. One kind of the solvent may be used, or two or more kinds thereof may be used in mixture.

### [Other Components]

The inventive resist composition may further contain a surfactant. As the surfactant, a fluorine-based and/or silicone-based surfactant is preferable. Specific examples of such a surfactant include surfactants disclosed in paragraph of US2008/0248425A1. Furthermore, it is also possible to use a surfactant disclosed in paragraph of US2008/0248425A1, other than the fluorine-based and/or silicone-based surfactants.

When the inventive resist composition contains the surfactant, the contained amount is preferably 0.0001 to 2 mass % of all the solid contents. One kind of the surfactant may be used, or two or more kinds thereof may be used in combination.

The inventive resist composition may further contain at least one selected from a radical scavenger, a crosslinking agent, a carboxy-group-containing compound, and other hypervalent iodine compounds. In this manner, the photoreaction during photolithography can be controlled, and sensitivity can be adjusted.

Specific examples of the radical scavenger include hindered phenols, quinones, hindered amines, and thiol compounds. Specific examples of the hindered phenols include dibutylhydroxytoluene (BHT) and 2,2′-methylenebis(4-methyl-6-tert-butylphenol). Specific examples of the quinones include 4-methoxyphenol (methoquinone) and hydroquinone. Specific examples of the hindered amines include 2,2,6,6-tetramethylpiperidine-N-oxy radical. Specific examples of the thiols include dodecanethiol and hexadecanethiol.

When the inventive resist composition contains the radical scavenger, the contained amount is preferably 0.01 to 10 mass % of all the solid contents. One kind of the radical scavenger may be used, or two or more kinds thereof may be used in combination.

Specific examples of the crosslinking agent include compounds having a carbon-carbon unsaturated bond as a functional group, such as a vinyl group, a (meth)acrylate group, an allyl group, an alkynyl group, and an aromatic ring. Specific examples of compounds having a vinyl group include linear alkenes, branched alkenes, and cyclic alkenes, each optionally having a substituent. Specific examples of compounds having a (meth)acrylate group include acrylic acid, methacrylic acid, acrylic acid ester, and methacrylic acid ester, each optionally having a substituent. Specific examples of compounds having an allyl group include allyl alcohol, allyl ether, allyl ester, allyl amide, allylamine, and allyl-group-containing isocyanurates, each optionally having a substituent. Specific examples of compounds having an alkynyl group include linear alkynes, branched alkynes, cyclic alkynes, alkynyl alcohols, alkynyl ethers, alkynyl esters, alkynyl amides, alkynyl amines, and alkynyl-group-containing isocyanurates, each optionally having a substituent. Specific examples of compounds having an aromatic ring include arenes, heteroarenes, styrene, stilbene, phenylacetylene, acenaphthylene, and chalcone, each optionally having a substituent. The crosslinking agent may have only one of the functional groups, or may have a plurality of the groups. The number of the functional groups contained in the crosslinking agent is preferably 1 or more and 10 or less, more preferably 2 or more and 8 or less.

When the inventive resist composition contains the crosslinking agent, the contained amount is preferably 0.01 to 50 mass % of all the solid contents. One kind of the crosslinking agent may be used, or two or more kinds thereof may be used in combination.

The inventive resist composition may contain, as an optional component, a carboxy-group-containing compound. When the carboxy-group-containing compound is contained, the carboxy-group-containing compound is preferably a polymer including a repeating unit represented by the following general formula (2) or a compound represented by the following general formula (3).

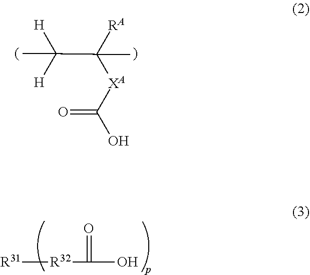

In the formulae, R^{A} represents a hydrogen atom, a halogen atom, a methyl group, or a trifluoromethyl group;
X^{A} represents a single bond, a phenylene group, a naphthylene group, or *—C(═O)—O—X^{A1}—, X^{A1} representing a saturated hydrocarbylene group having 1 to 10 carbon atoms, a phenylene group, or a naphthylene group, the saturated hydrocarbylene group optionally containing a hydroxy group, an ether bond, an ester bond, or a lactone ring, and “*” representing an attachment point to the carbon atom of the main chain;
“p” represents 1, 2, 3, or 4;
R³¹ represents a p-valent hydrocarbon group having 1 to 40 carbon atoms or a p-valent heterocyclic group having 2 to 40 carbon atoms, when “p” is 2, the R³¹ optionally being an ether bond, a carbonyl group, an azo group, a thioether bond, a carbonate bond, a carbamate bond, a sulfinyl group, or a sulfonyl group, part or all of hydrogen atoms of the p-valent hydrocarbon group or the p-valent heterocyclic group optionally being substituted with a group containing a heteroatom, and part of —CH₂— of the p-valent hydrocarbon group optionally being substituted with a group containing a heteroatom; and
R³² represents a single bond or a hydrocarbylene group having 1 to 20 carbon atoms, part or all of hydrogen atoms of the hydrocarbylene group optionally being substituted with a group containing a heteroatom, part of —CH₂— of the hydrocarbylene group optionally being substituted with a group containing a heteroatom, and when “p” is 2, 3, or 4, the R³²'s being identical to or different from each other.

In the general formula (2), R^{A} represents a hydrogen atom, a halogen atom, a methyl group, or a trifluoromethyl group. X^{A} represents a single bond, a phenylene group, a naphthylene group, or *—C(═O)—O—X^{A1}—. X^{A1} represents a saturated hydrocarbylene group having 1 to 10 carbon atoms, a phenylene group, or a naphthylene group, the saturated hydrocarbylene group optionally containing a hydroxy group, an ether bond, an ester bond, or a lactone ring. “*” represents an attachment point to the carbon atom of the main chain.

In the general formula (3), “p” represents 1, 2, 3, or 4.

In the general formula (3), R³¹ represents a p-valent hydrocarbon group having 1 to 40 carbon atoms or a p-valent heterocyclic group having 2 to 40 carbon atoms, when “p” is 2, the R³¹ optionally being an ether bond, a carbonyl group, an azo group, a thioether bond, a carbonate bond, a carbamate bond, a sulfinyl group, or a sulfonyl group. Part or all of the hydrogen atoms of the p-valent hydrocarbon group or the p-valent heterocyclic group may be substituted with a group containing a heteroatom, and part of the —CH₂— of the p-valent hydrocarbon group may be substituted with a group containing a heteroatom.

In the general formula (3), R³² represents a single bond or a hydrocarbylene group having 1 to 10 carbon atoms, part or all of the hydrogen atoms of the hydrocarbylene group optionally being substituted with a group containing a heteroatom, and part of the —CH₂— of the hydrocarbylene group optionally being substituted with a group containing a heteroatom. When “p” is 2, 3, or 4, the R³²'s may be identical to or different from each other.

The p-valent hydrocarbon group represented by R³¹ may be saturated or unsaturated, and may be linear, branched, or cyclic. The p-valent hydrocarbon group is a group obtained by “p” hydrogen atoms being removed from a hydrocarbon. Examples of the hydrocarbon include alkanes having 1 to 40 carbon atoms, alkenes having 2 to 40 carbon atoms, alkynes having 2 to 40 carbon atoms, cyclic saturated hydrocarbons having 3 to 40 carbon atoms, cyclic unsaturated hydrocarbons having 3 to 40 carbon atoms, and aromatic hydrocarbons having 6 to 40 carbon atoms.

Examples of the alkanes having 1 to 40 carbon atoms include methane, ethane, propane, butane, pentane, hexane, heptane, octane, nonane, decane, and structural isomers thereof.

Examples of the alkenes having 2 to 40 carbon atoms include ethylene, propylene, butene, pentene, hexene, heptene, octene, nonene, decene, and structural isomers thereof.

Examples of the alkynes having 2 to 40 carbon atoms include acetylene, propyne, butyne, pentyne, hexyne, heptyne, octyne, nonyne, decyne, and structural isomers thereof.

Examples of the cyclic saturated hydrocarbons having 3 to 40 carbon atoms include cyclopropane, cyclobutane, cyclopentane, cyclohexane, cycloheptane, cyclooctane, adamantane, and norbornane.

Examples of the cyclic unsaturated hydrocarbons having 3 to 40 carbon atoms include cyclopropene, cyclobutene, cyclopentene, cyclohexene, cycloheptene, cyclooctene, and norbornene.

Examples of the aromatic hydrocarbons having 6 to 40 carbon atoms include benzene, naphthalene, and biphenyl.

The p-valent heterocyclic group represented by R³¹ is a group obtained by removing “p” hydrogen atoms from a heterocyclic compound. Examples of the heterocyclic compound include furan, pyridine, pyrazole, and thiazolidine.

Part or all of the hydrogen atoms of the p-valent hydrocarbon group or the p-valent heterocyclic group may be substituted with a group containing a heteroatom such as an oxygen atom, a sulfur atom, a nitrogen atom, or a halogen atom. The resulting p-valent hydrocarbon group or p-valent heterocyclic group may contain a hydroxy group, a cyano group, a fluorine atom, a chlorine atom, a bromine atom, an iodine atom, etc. Furthermore, part of the —CH₂-constituting the p-valent hydrocarbon group may be substituted with a group containing a heteroatom such as an oxygen atom, a sulfur atom, or a nitrogen atom. The resulting p-valent hydrocarbon group may contain a carbonyl group, an ether bond, a thioether bond, an ester bond, a sulfonic acid ester bond, a carbonate bond, a carbamate bond, a lactone ring, a sultone ring, a carboxylic anhydride (—C(═O)—O—C(═O)—), etc.

The hydrocarbylene group represented by R³² may be saturated or unsaturated, and may be linear, branched, or cyclic. Specific examples thereof include: alkanediyl groups having 1 to 20 carbon atoms, such as a methanediyl group, an ethane-1,1-diyl group, an ethane-1,2-diyl group, a propane-1,2-diyl group, a propane-1,3-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, and a dodecane-1,12-diyl group; cyclic saturated hydrocarbylene groups having 3 to 20 carbon atoms, such as a cyclopentanediyl group, a cyclohexanediyl group, a norbornanediyl group, and an adamantanediyl group; unsaturated aliphatic hydrocarbylene groups having 2 to 20 carbon atoms, such as a vinylene group and a propene-1,3-diyl group; arylene groups having 6 to 20 carbon atoms, such as a phenylene group and a naphthylene group; and groups obtained by combining these groups. Furthermore, part or all of the hydrogen atoms of the hydrocarbylene group may be substituted with a group containing a heteroatom, such as an oxygen atom, a sulfur atom, a nitrogen atom, or a halogen atom, and part of the —CH₂-constituting the hydrocarbylene group may be substituted with a group containing a heteroatom such as an oxygen atom, a sulfur atom, or a nitrogen atom. The resulting hydrocarbylene group may contain a hydroxy group, a cyano group, a fluorine atom, a chlorine atom, a bromine atom, an iodine atom, a carbonyl group, an ether bond, a thioether bond, an ester bond, a sulfonic acid ester bond, a carbonate bond, a carbamate bond, a lactone ring, a sultone ring, a carboxylic anhydride, etc.

Among carboxylic acid compounds represented by the general formula (3), compounds in which “p” is 2, 3, or 4 are preferable. When such a carboxylic acid compound is mixed with the hypervalent iodine compound, a strong resist film having a high molecular weight can be easily formed, and therefore, such compounds are preferable from the viewpoints of etching resistance and developer resistance.

Specific examples of the repeating unit represented by the general formula (2) include the following, but are not limited thereto. Note that, in the following formulae, R^{A} is as defined above.

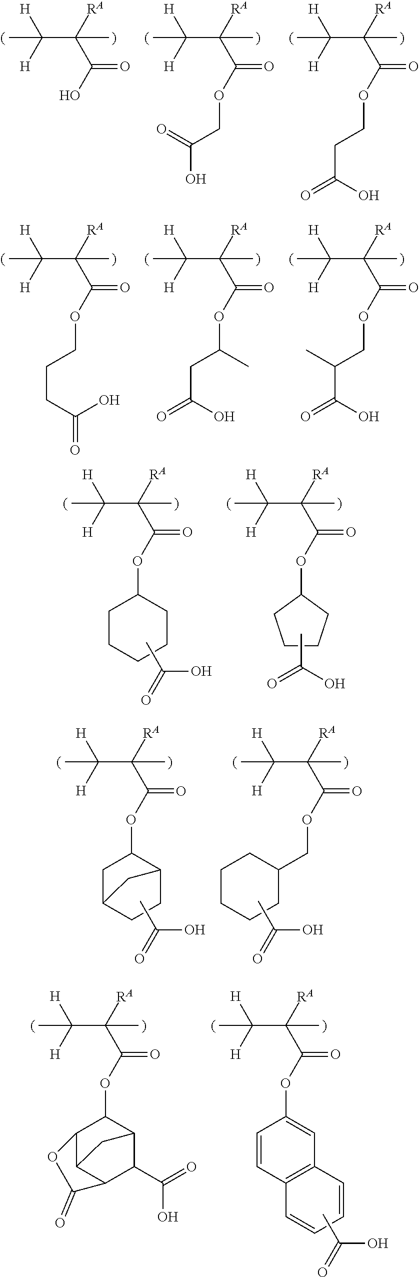

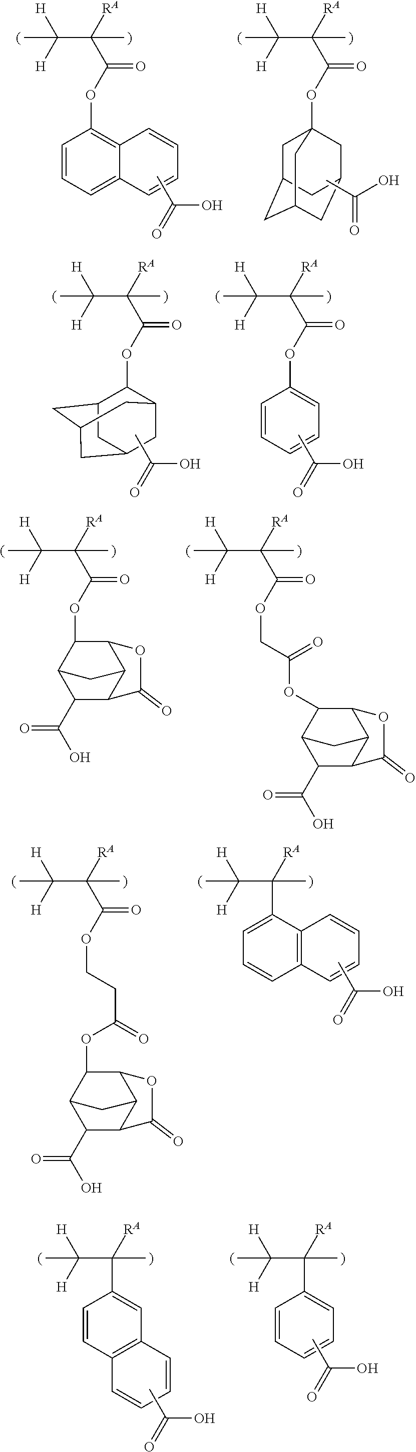

Examples of the carboxylic acid compound represented by the general formula (3) include the following, but are not limited thereto.

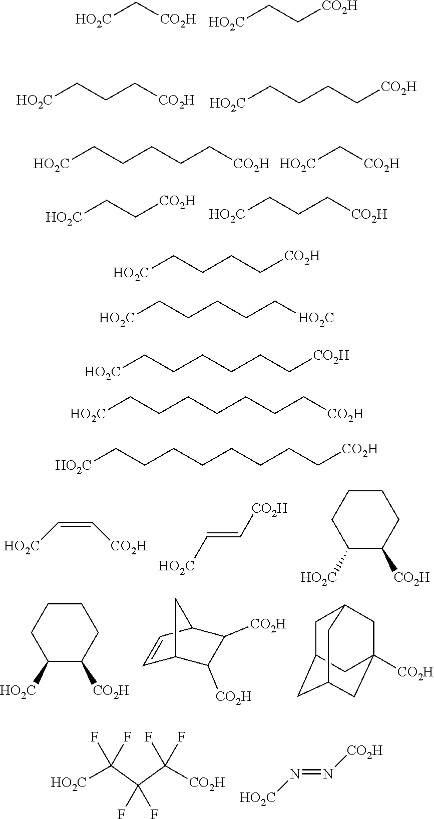

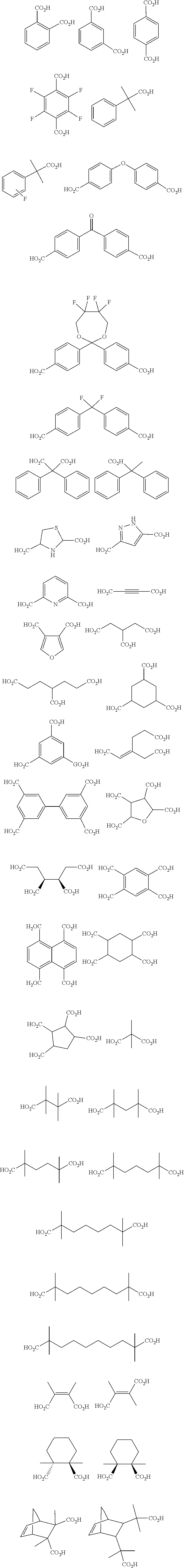

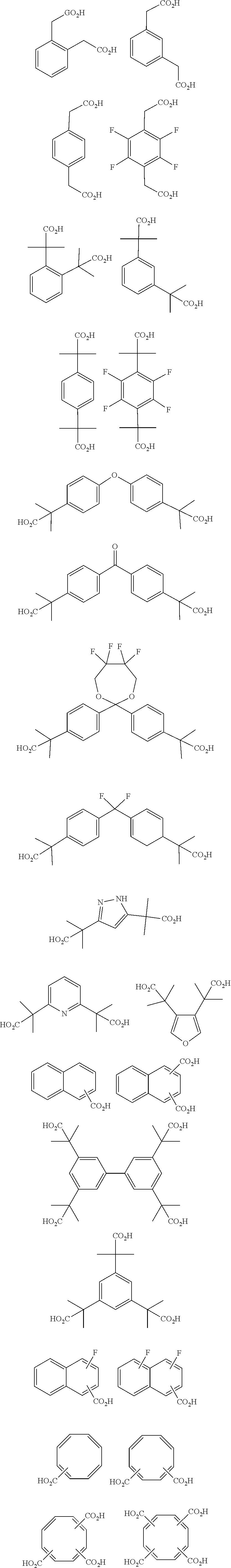

The polymer including the repeating unit represented by the general formula (2) may further include repeating units (hereinafter, also referred to as “other repeating units”) other than the repeating unit represented by the general formula (2). The other repeating units are not particularly limited, but those which may enhance the solubility of the polymer in a solvent are preferable, because of the polymer being hardly soluble when having only the repeating unit represented by the general formula (2). As the other repeating units, repeating units having a cyclic structure and repeating units including a styrene skeleton, the units having a rigid skeleton being expected to have high etching resistance, are preferable.

Specific examples of the other repeating units include the following, but are not limited thereto. Note that, in the following formulae, R^{A} is as defined above and each X^{B} independently represents —CH₂— or —O—.

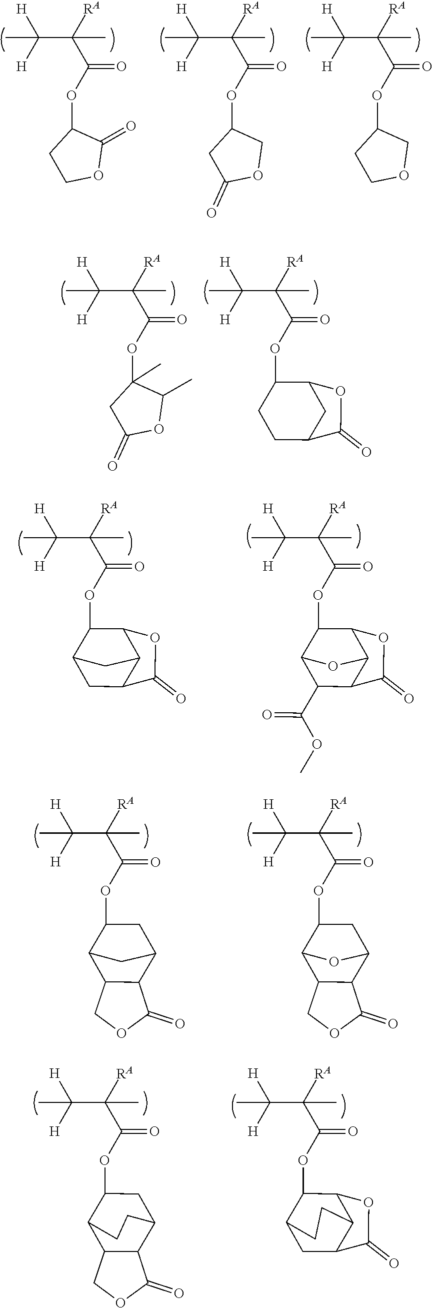

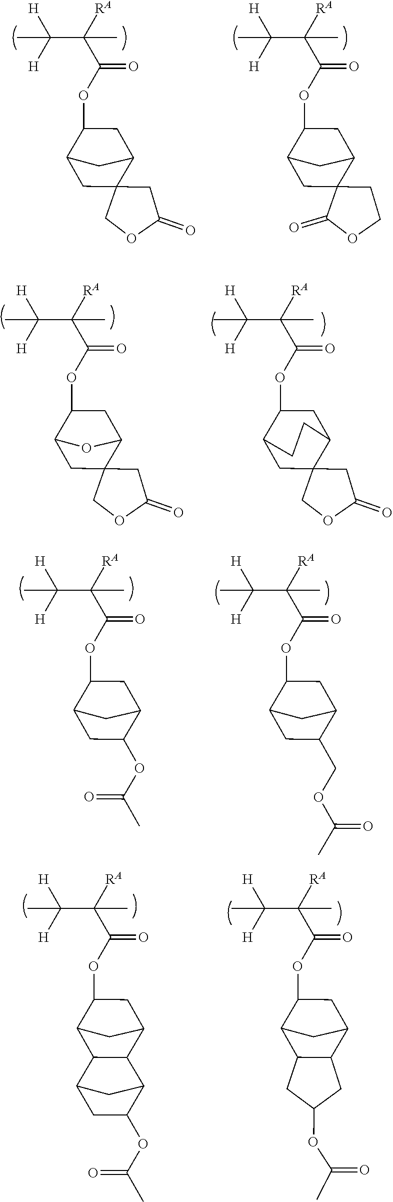

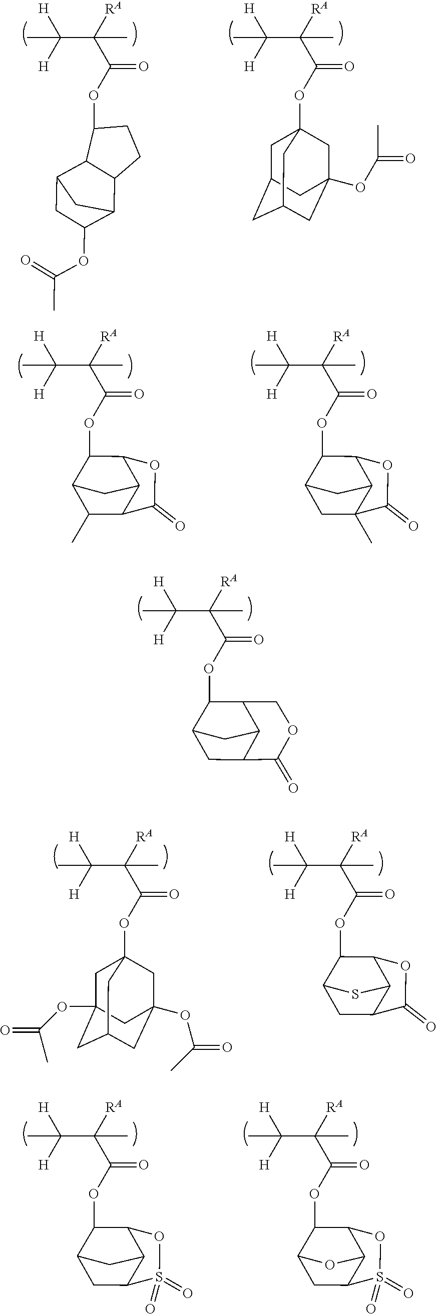

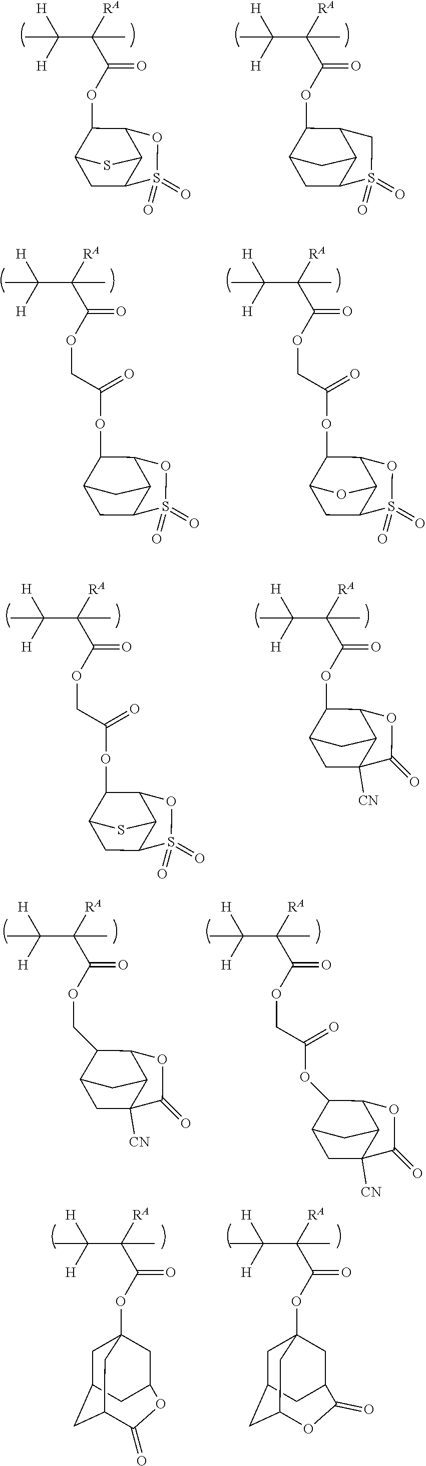

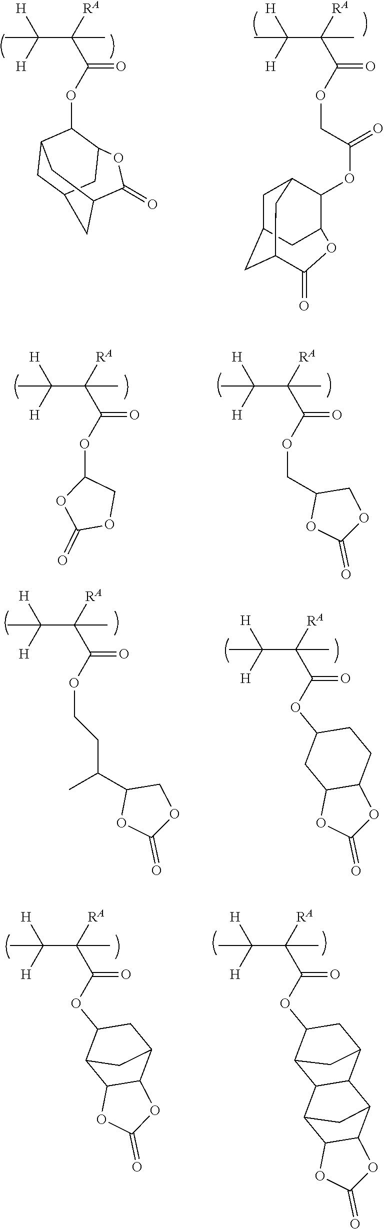

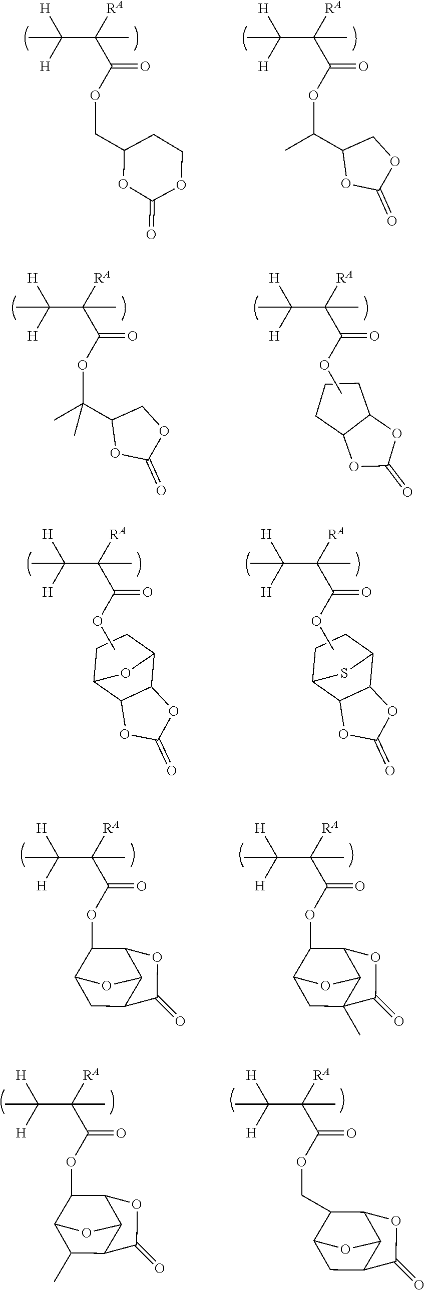

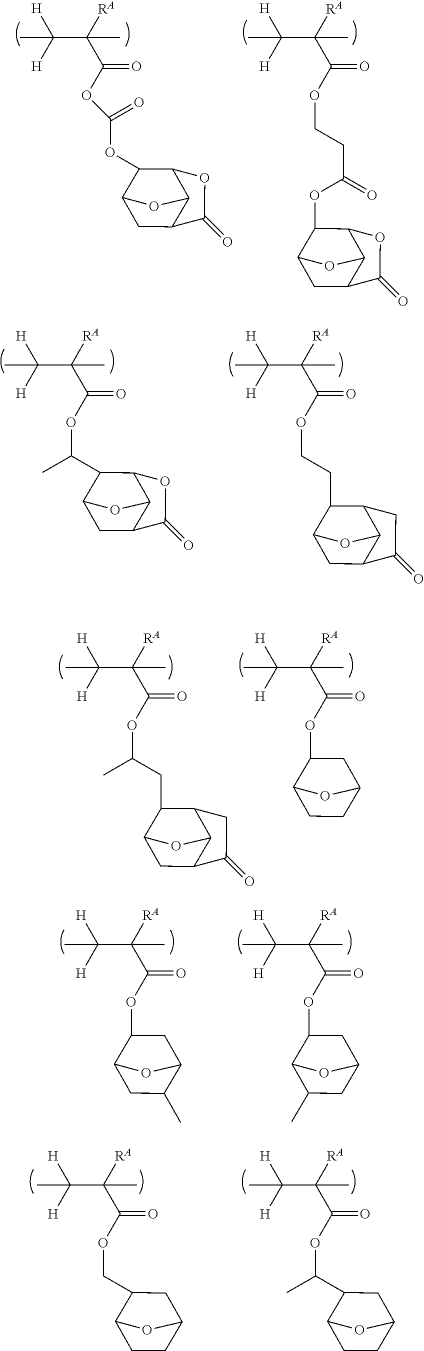

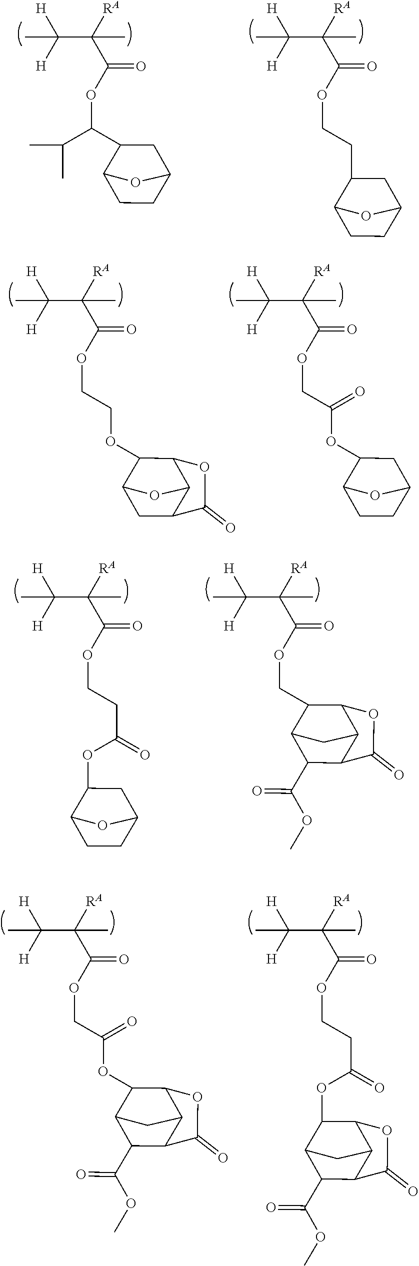

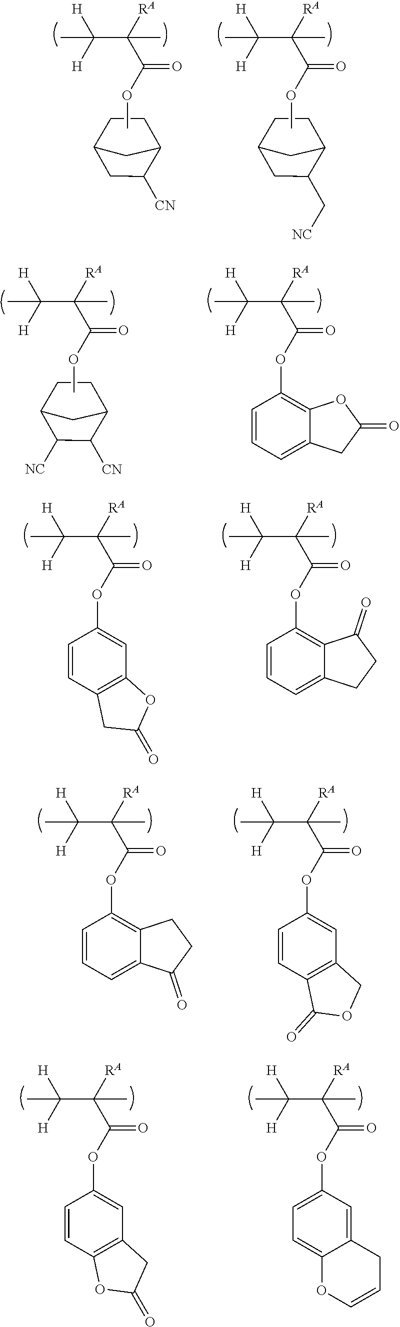

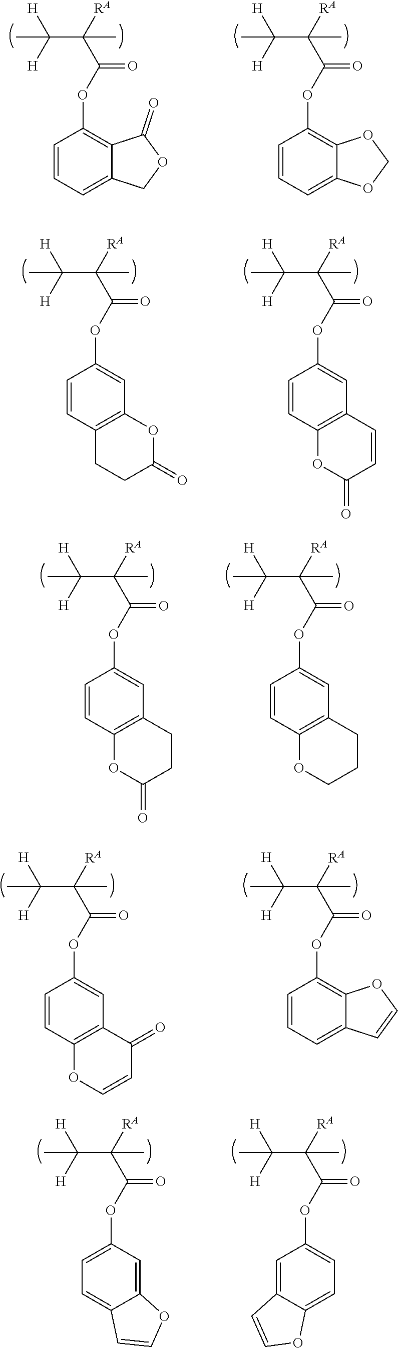

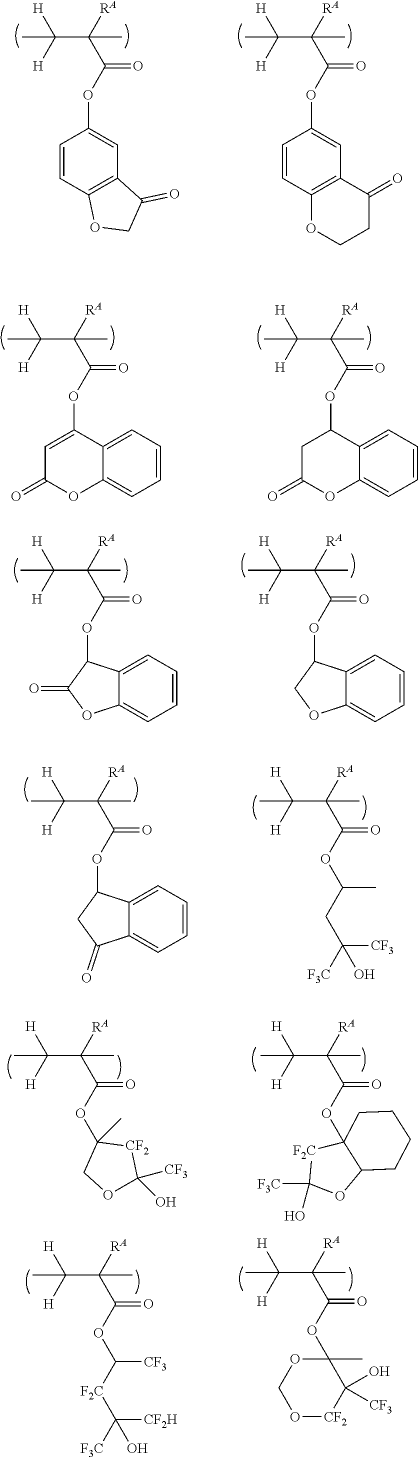

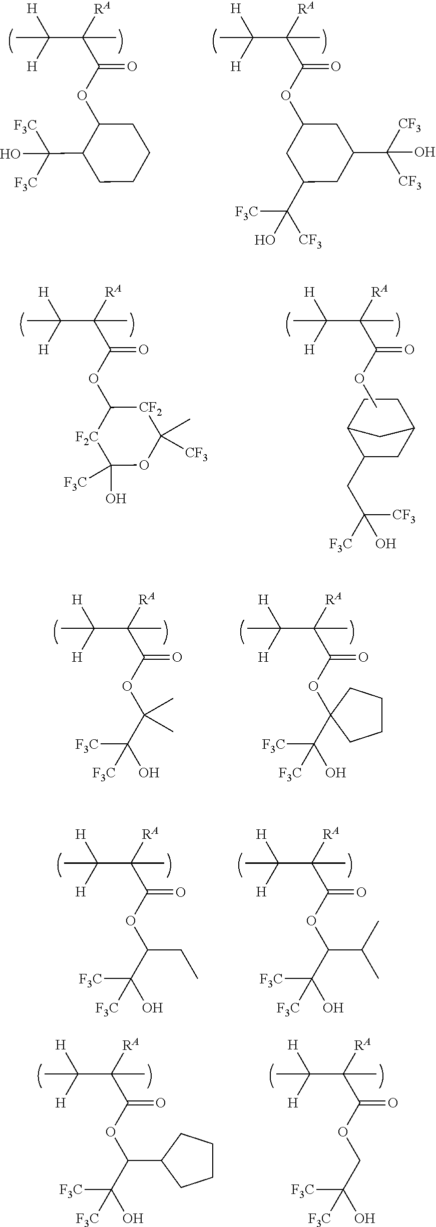

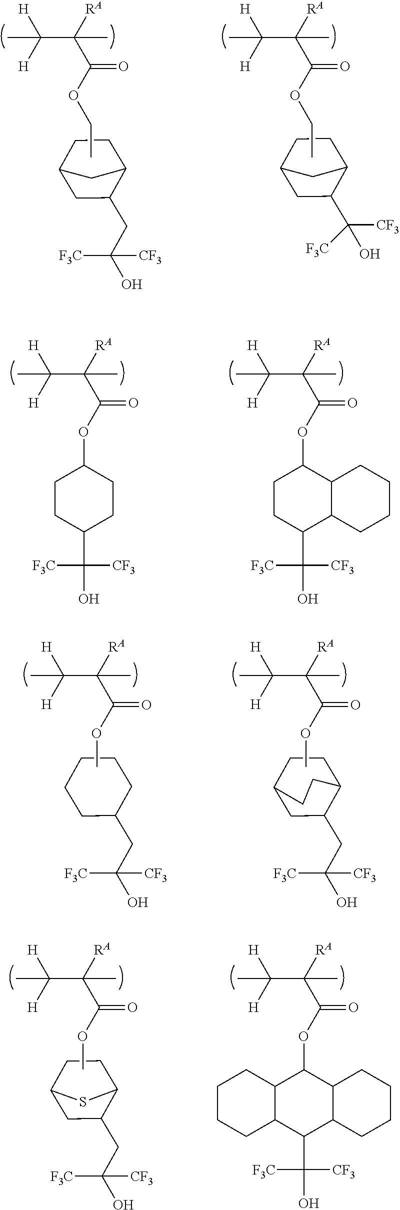

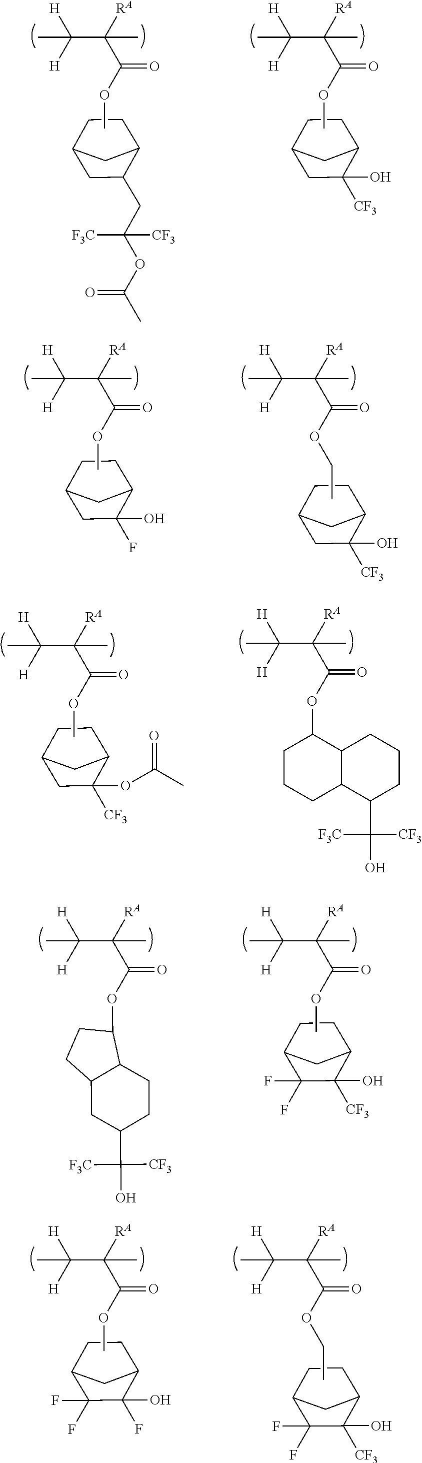

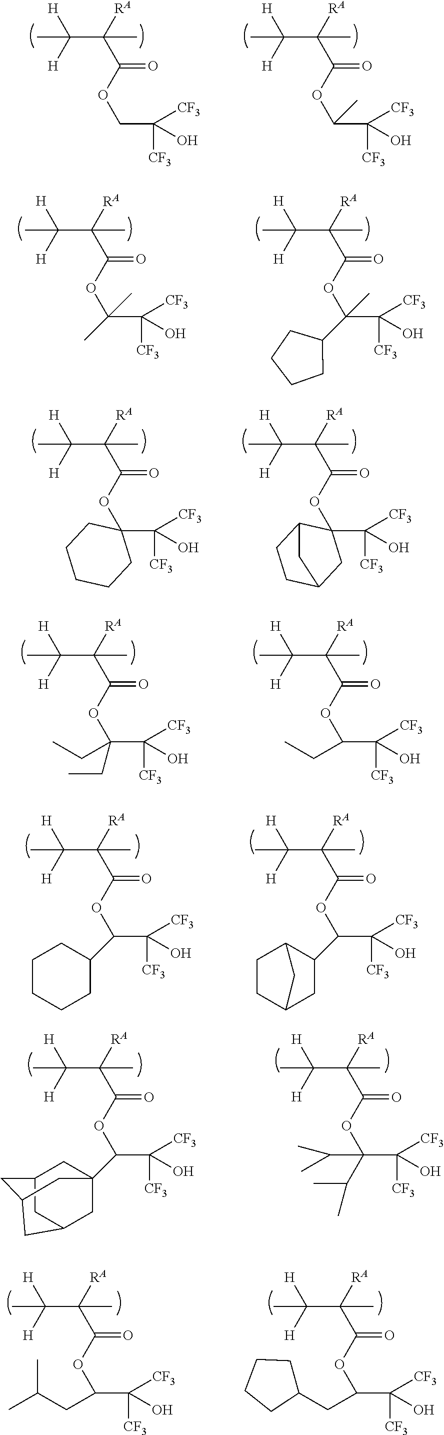

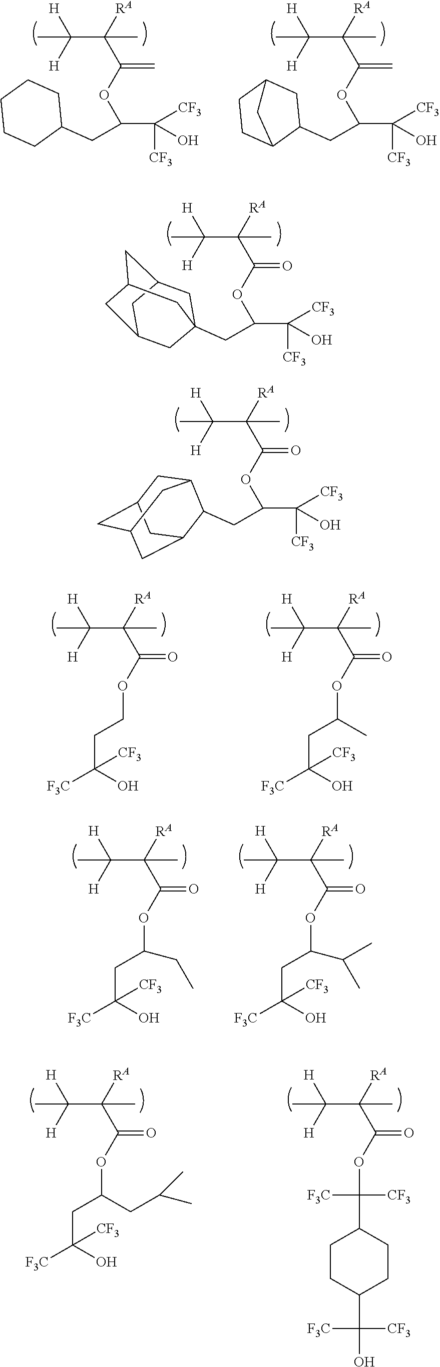

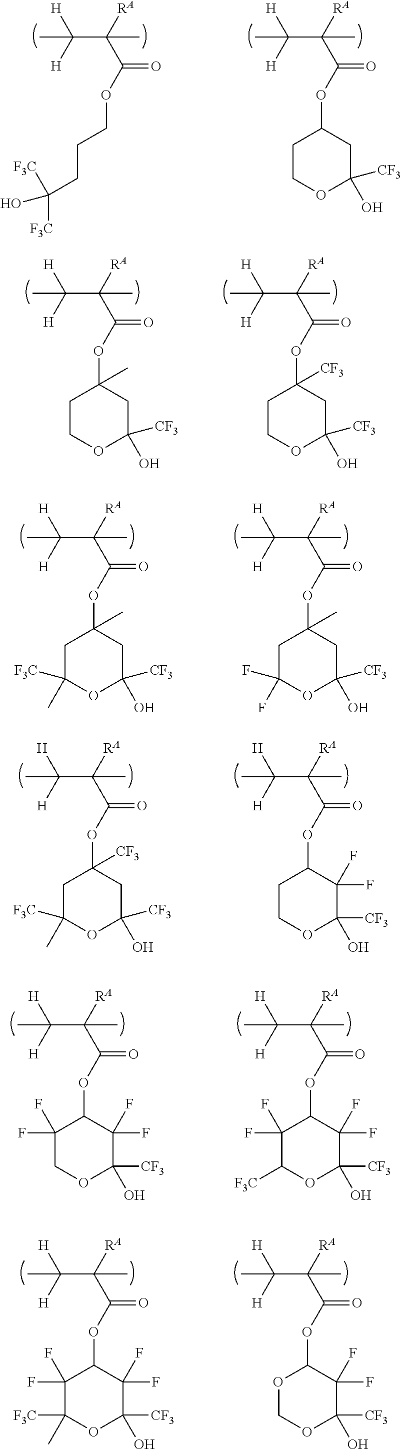

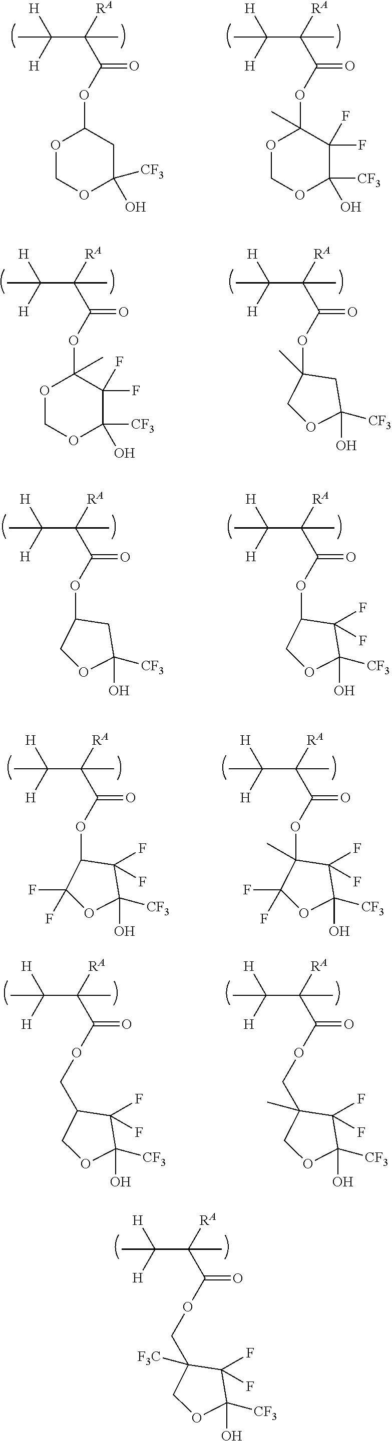

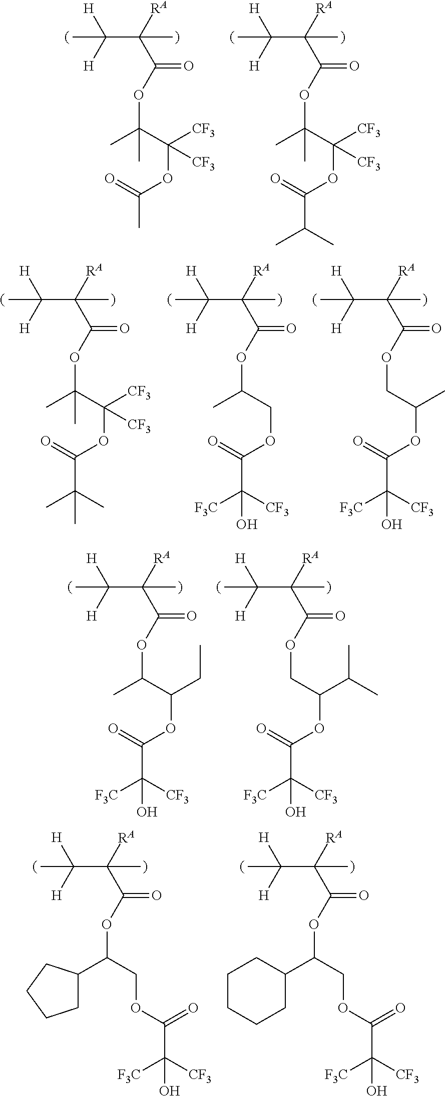

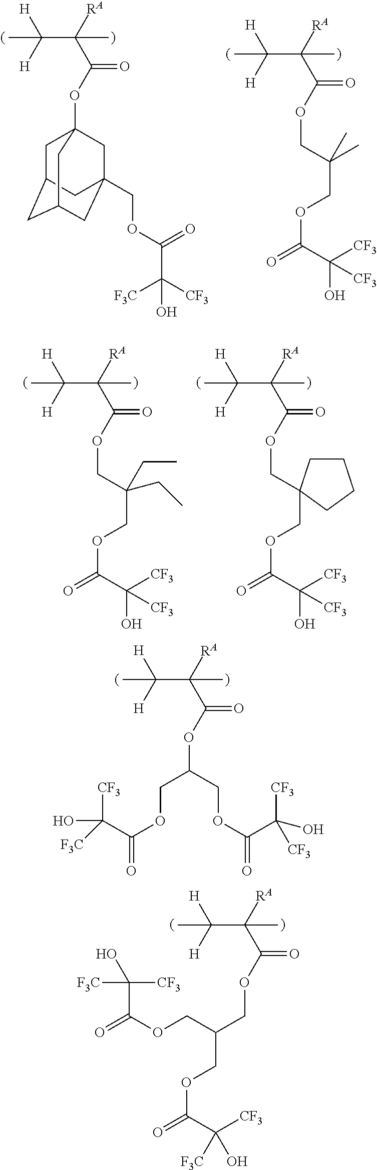

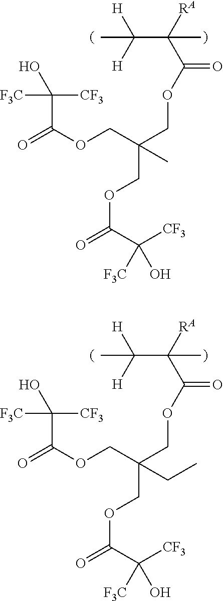

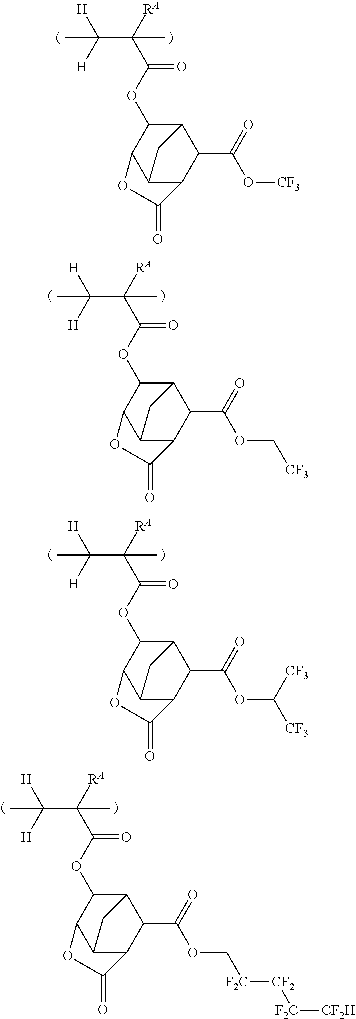

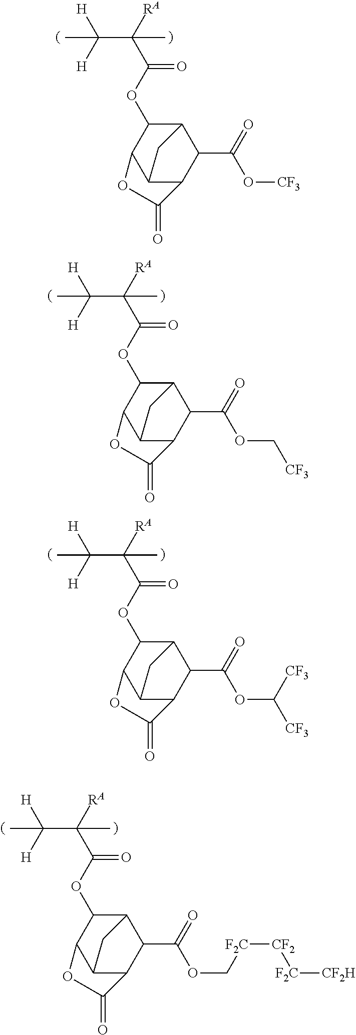

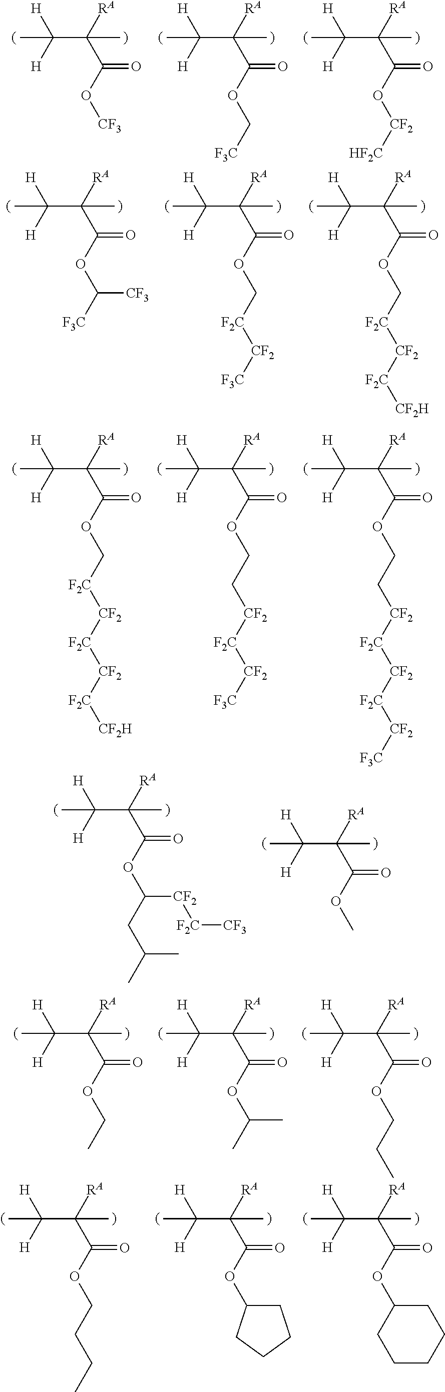

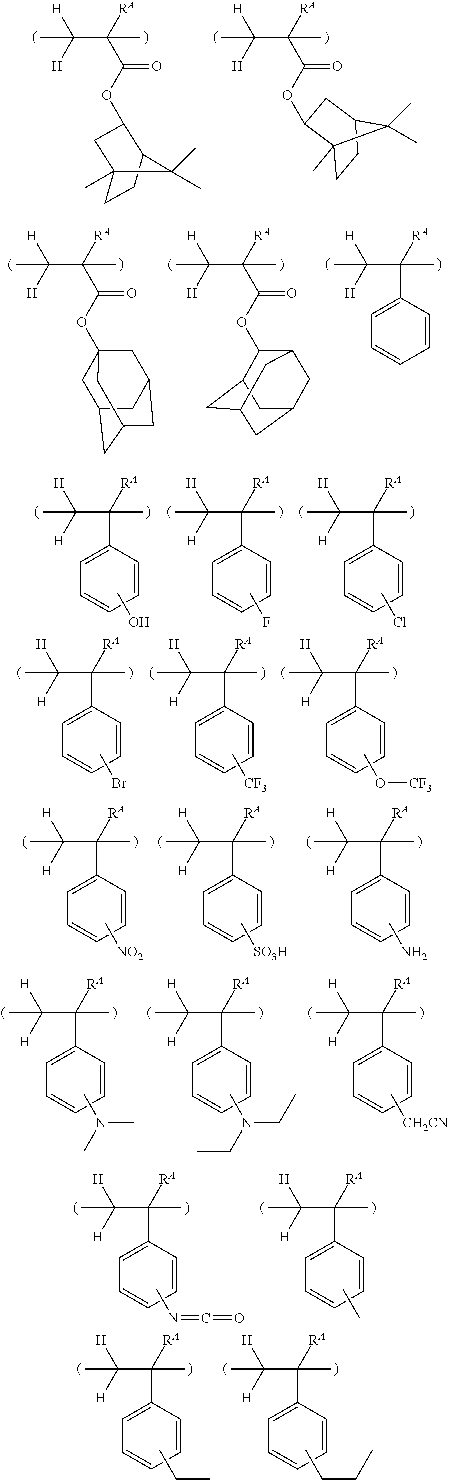

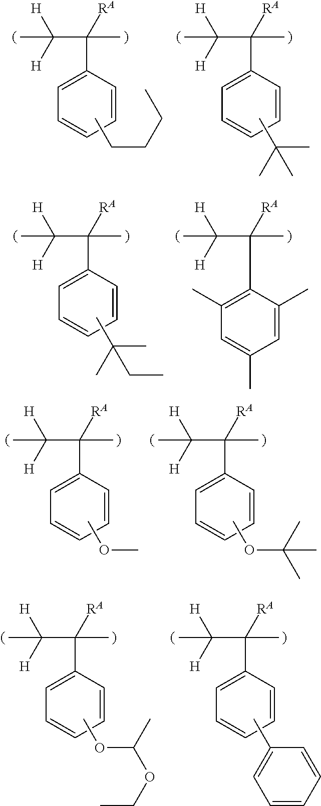

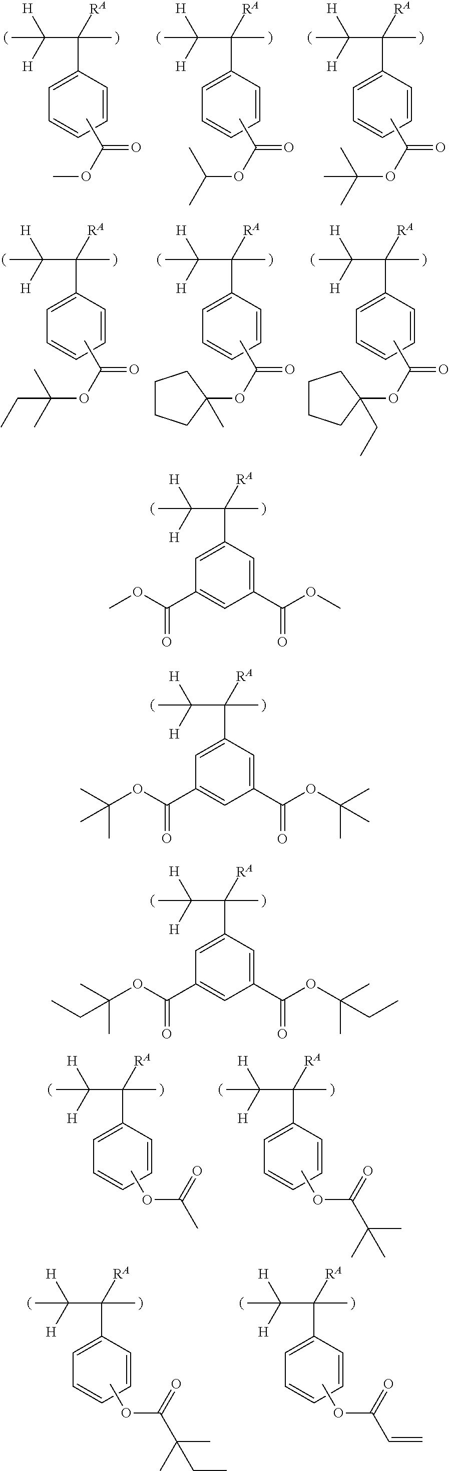

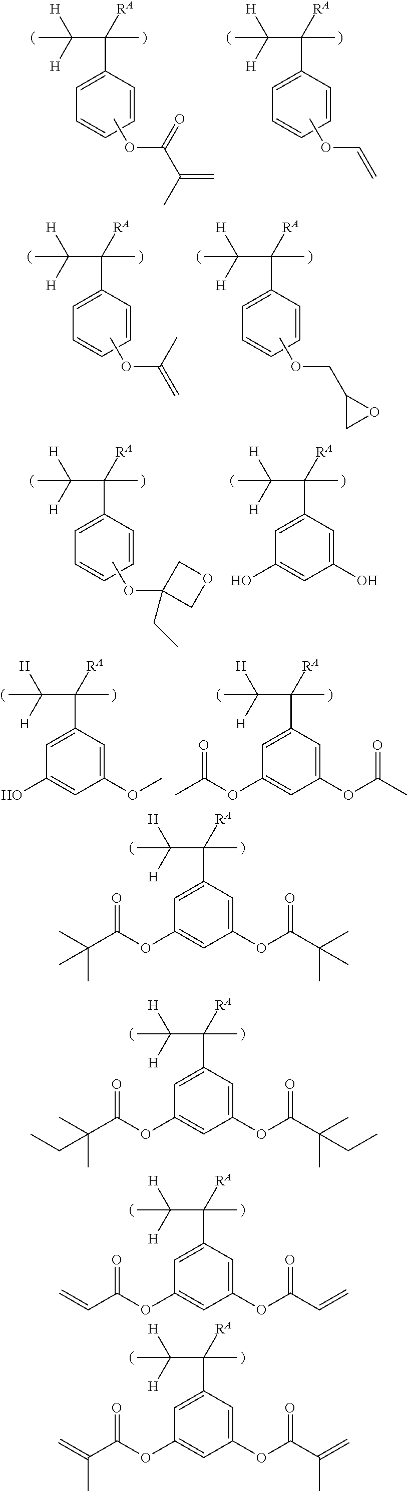

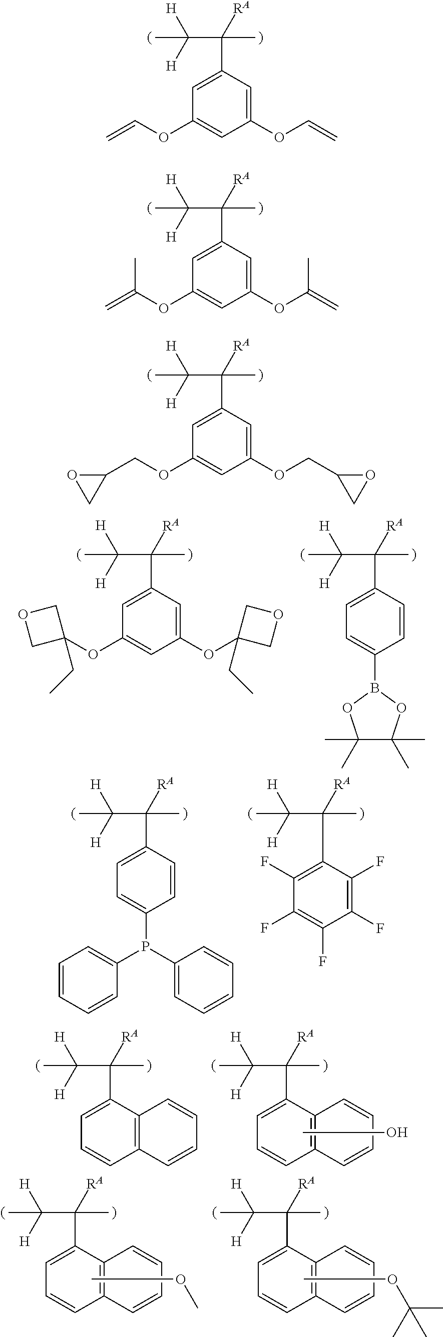

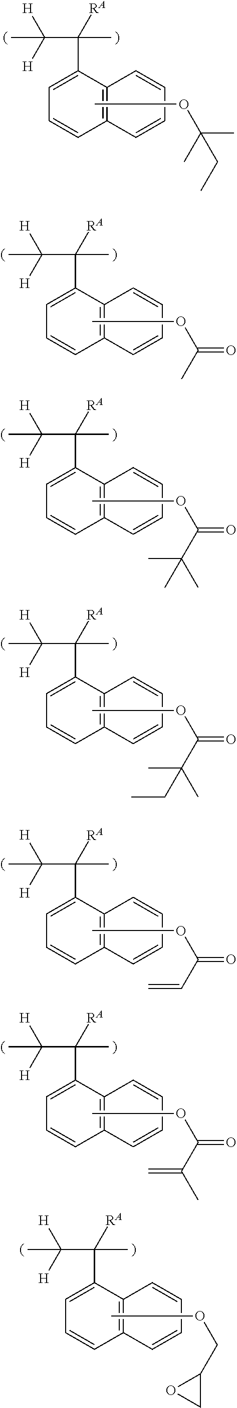

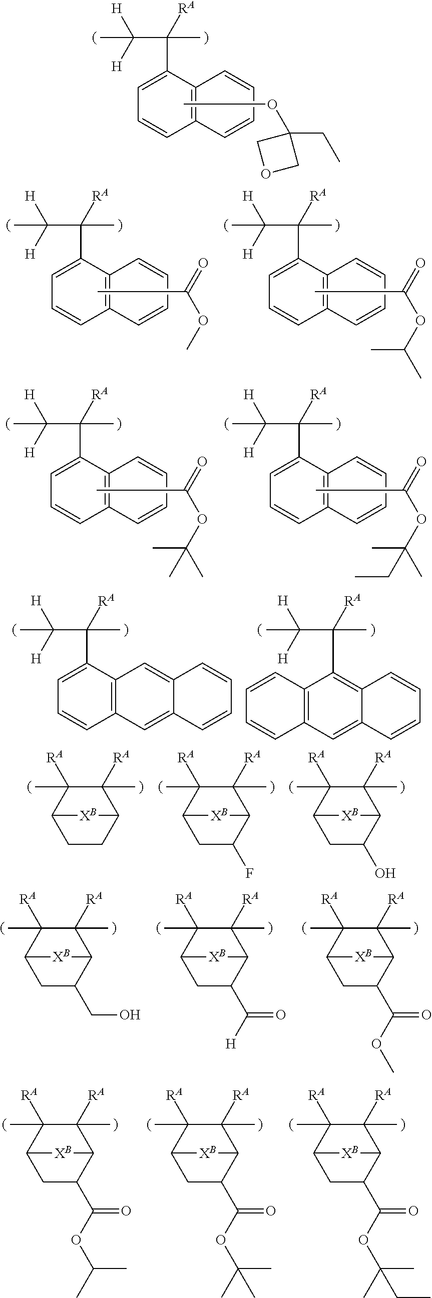

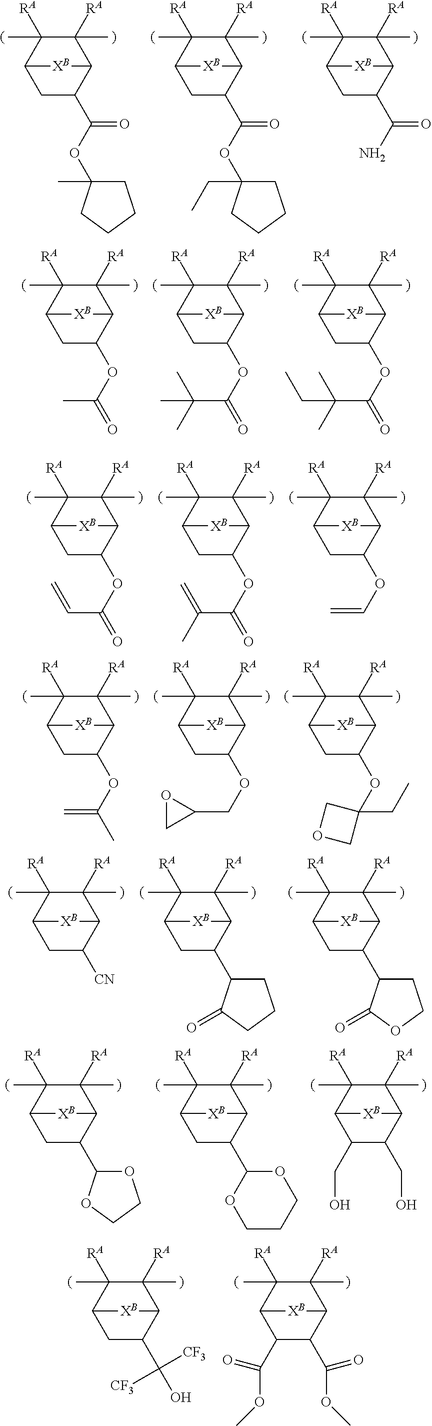

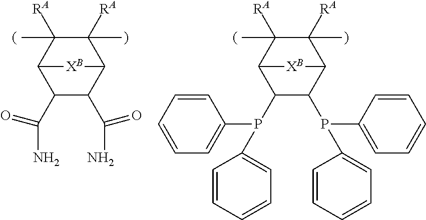

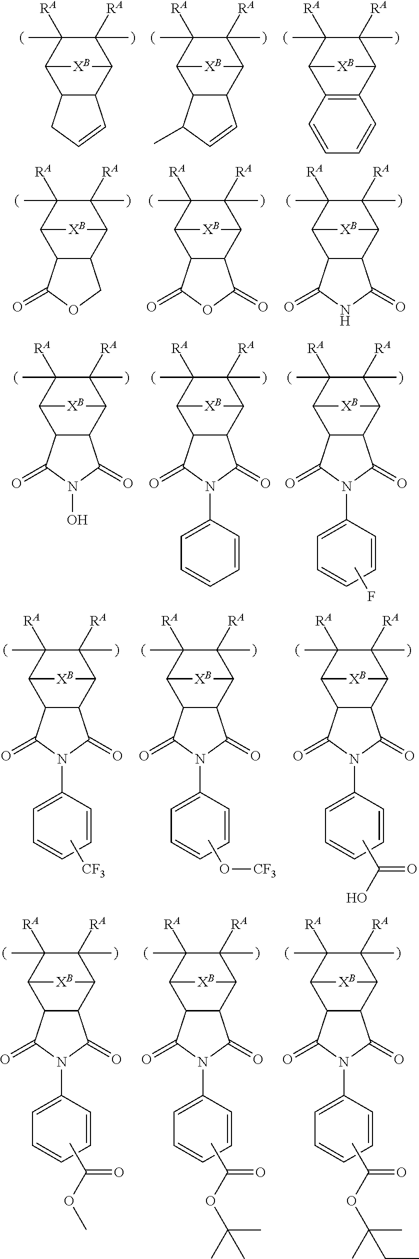

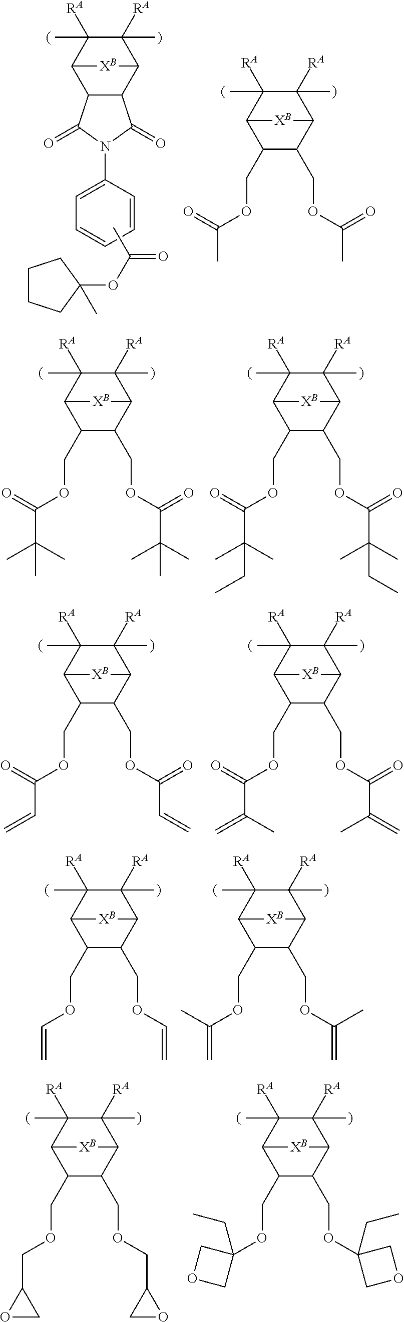

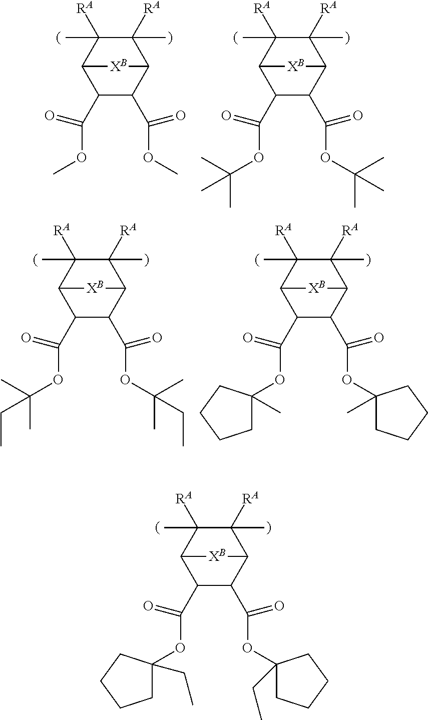

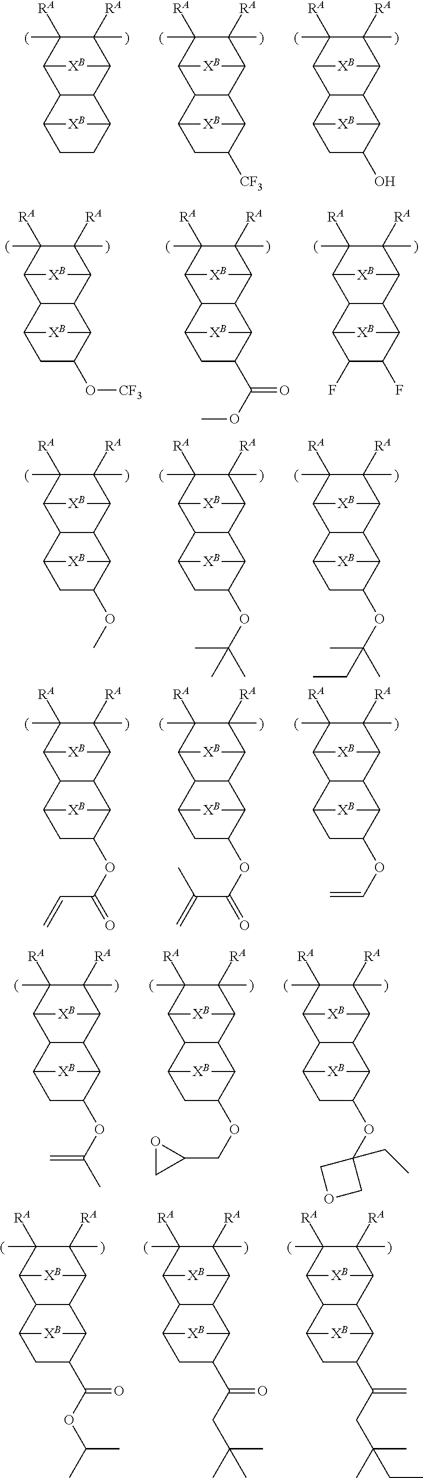

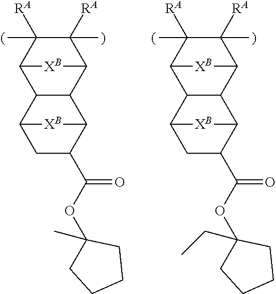

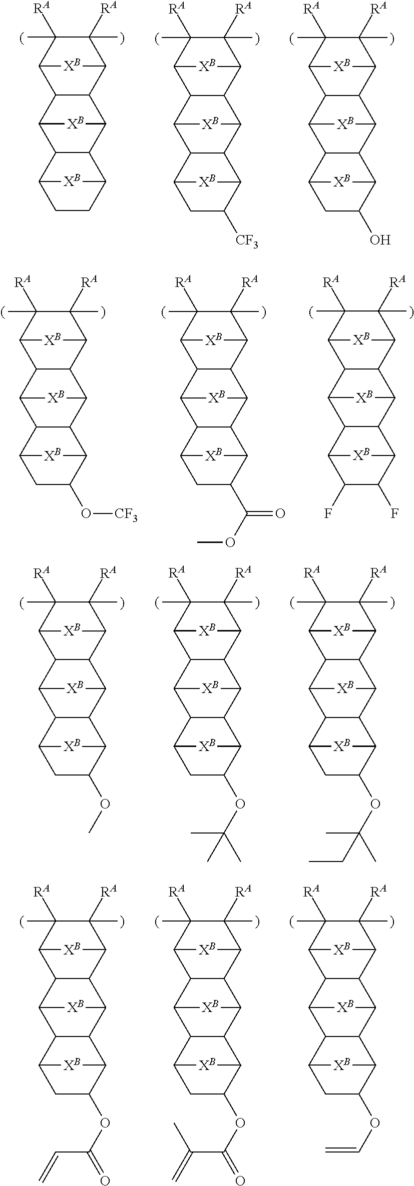

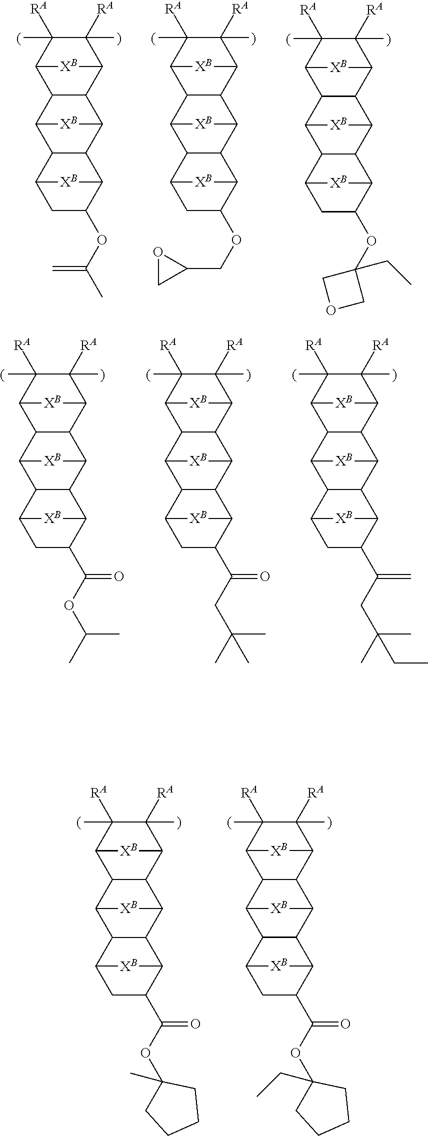

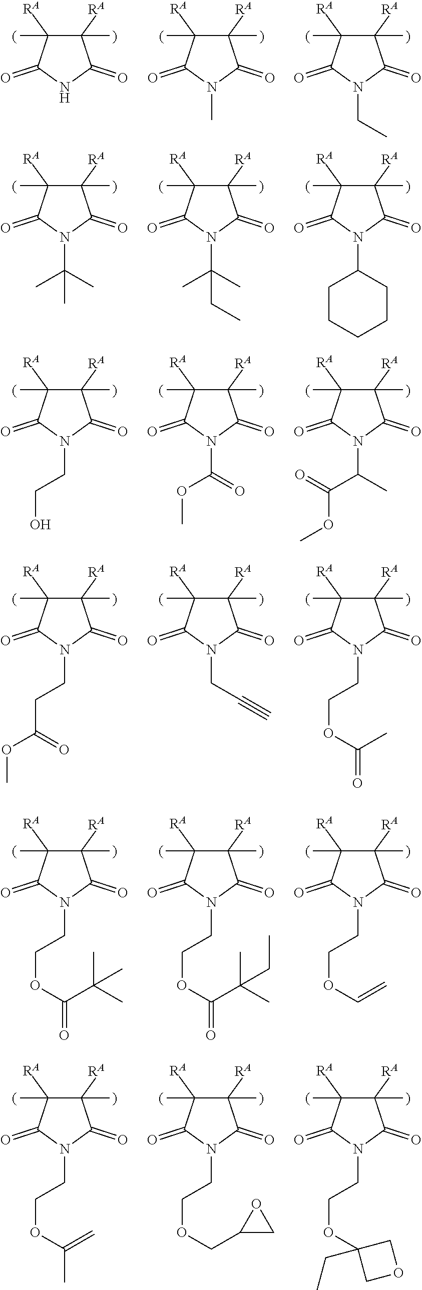

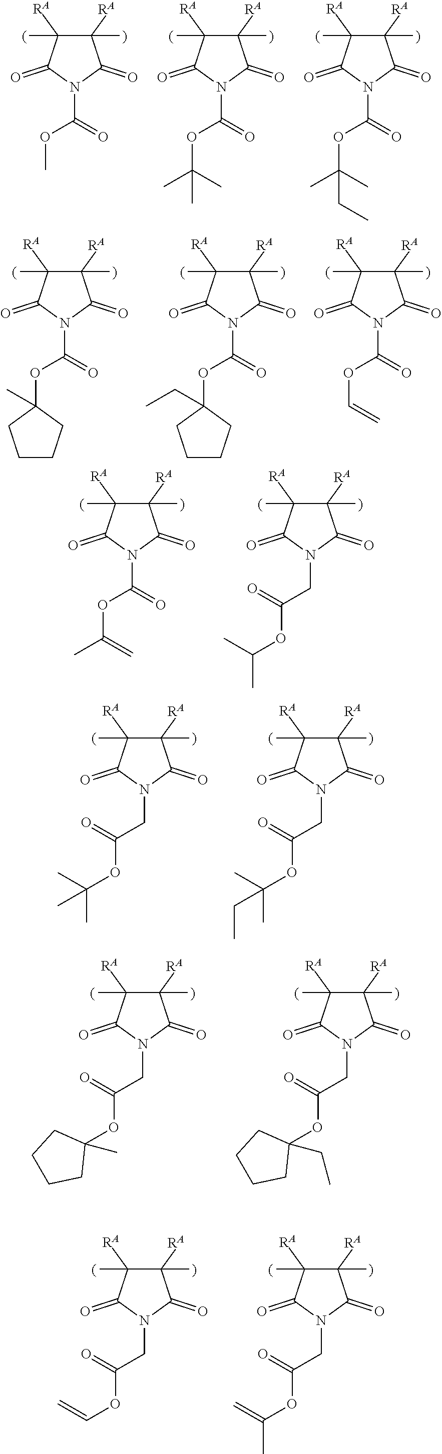

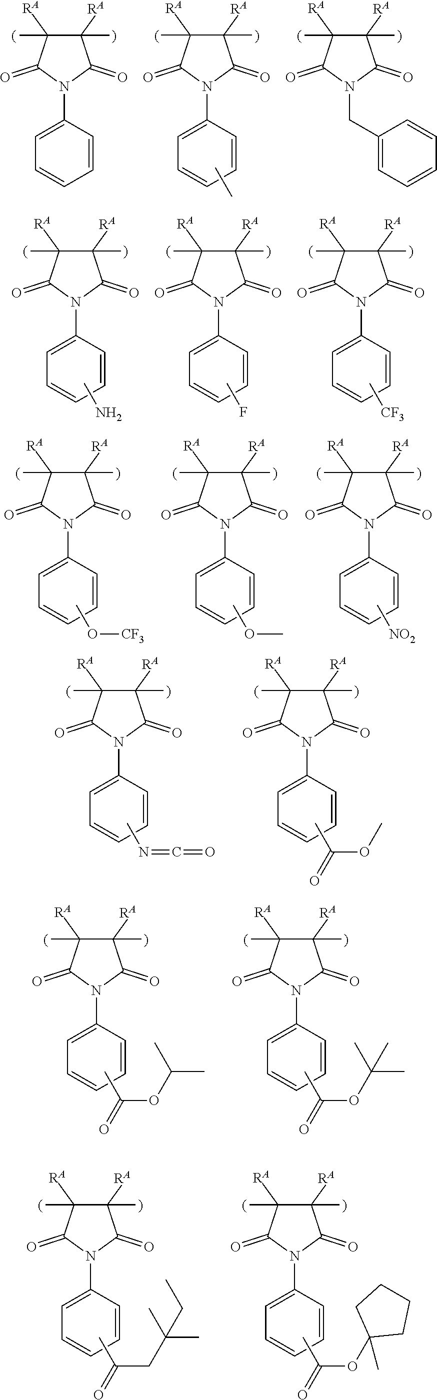

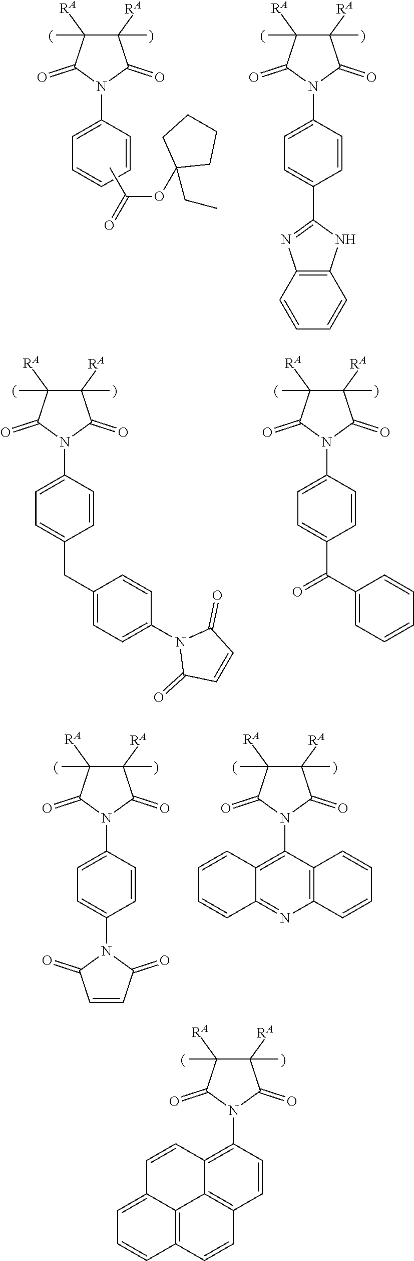

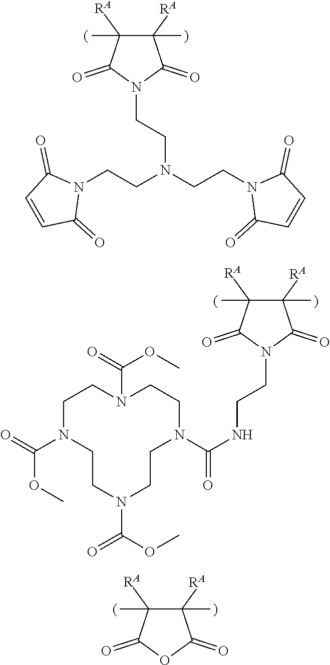

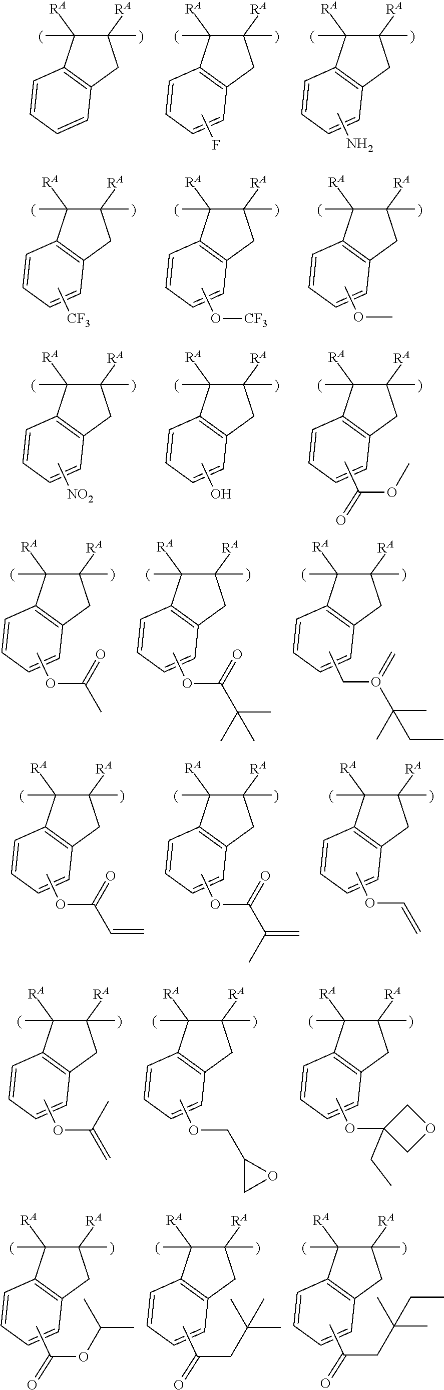

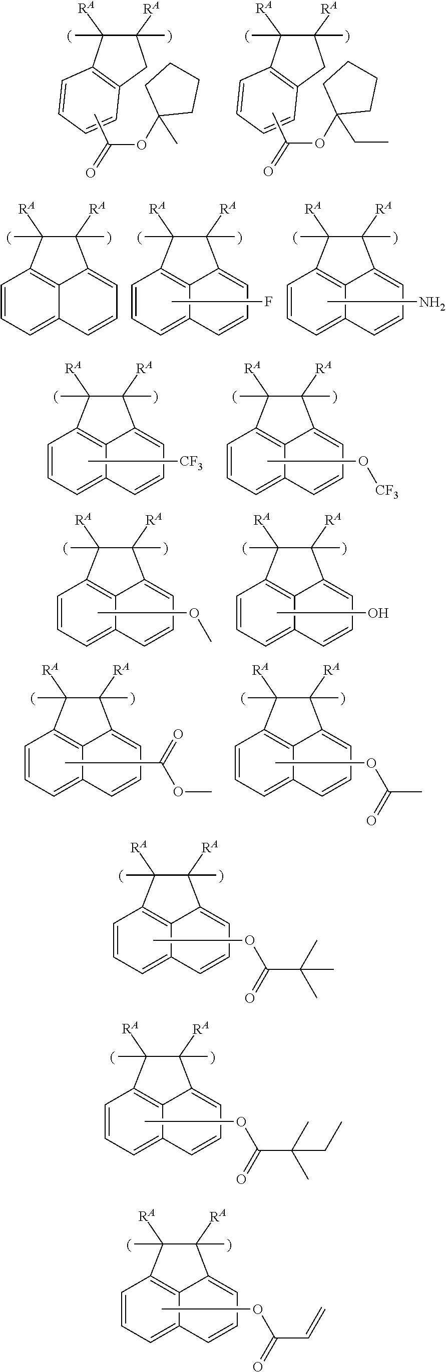

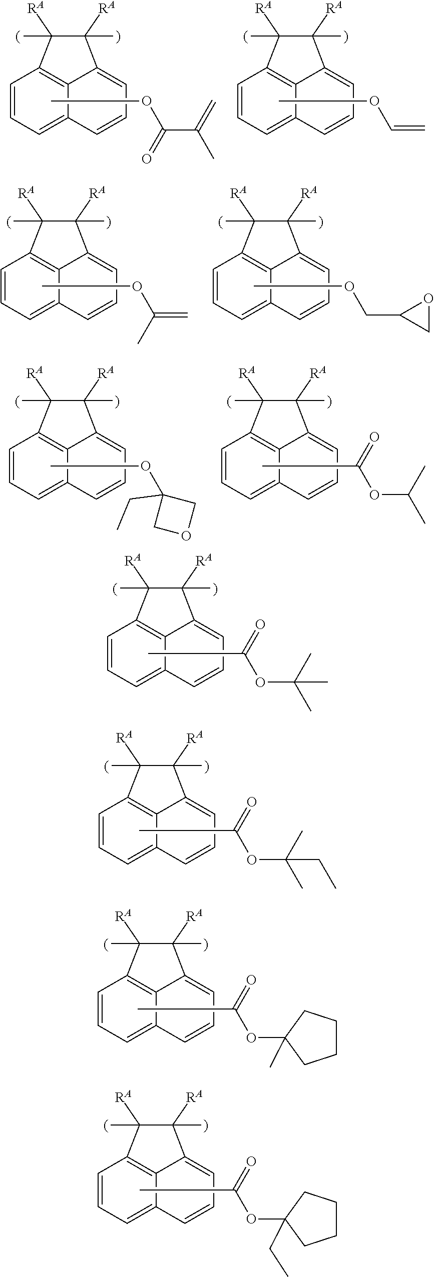

When the inventive resist composition contains a carboxy-group-containing compound as an optional component, the content ratio of the hypervalent iodine compound represented by the general formula (1) to the carboxy-group-containing compound (when the carboxy-group-containing compound is a carboxy-group-containing polymer, the content ratio of the hypervalent iodine compound represented by the general formula (1) to the repeating units having a carboxy group in the polymer) is preferably “hypervalent iodine compound represented by the general formula (1)”:“carboxy-group-containing compound”=100:0 to 50:50, more preferably 100:0 to 60:40, and further preferably 100:0 to 80:20 in molar ratio. One kind of the hypervalent iodine compound represented by the general formula (1) may be used, or two or more kinds thereof may be used in combination. Similarly, one kind of the carboxy-group-containing compound may be used, or two or more kinds thereof may be used in combination. When the carboxy-group-containing compound is a polymer, one kind thereof may be used, or two or more kinds thereof having different composition ratios, Mw, and/or Mw/Mn may be used in combination.

In the carboxy-group-containing polymer, the content ratio (molar ratio) of the repeating units having a carboxy group to the other repeating units is preferably “repeating units having a carboxy group”: “other repeating units”=10:90 to 90:10, more preferably 15:85 to 85:15, and further preferably 20:80 to 80:20.

The carboxy-group-containing polymer preferably has a weight-average molecular weight (Mw) of 1,000 to 500,000, more preferably 3,000 to 100,000. Note that, in the present invention, weight-average molecular weight Mw and number-average molecular weight Mn are values measured in terms of polystyrene by gel permeation chromatography (GPC) using tetrahydrofuran (THF) as an eluent.

Molecular weight distribution Mw/Mn can be calculated from the Mw and the Mn thus obtained. Furthermore, if the carboxy-group-containing polymer has a wide molecular weight distribution (Mw/Mn), polymers having a molecular weight lower or higher than the Mw are present, and therefore, there are risks that foreign substances may be found on the pattern after exposure, and the pattern shape may be degraded. Accordingly, as pattern rule is miniaturized, the influence of Mw and Mw/Mn is likely to be greater, and therefore, to obtain a resist composition that can be used suitably for a fine pattern size, the carboxy-group-containing polymer preferably has a narrow dispersity Mw/Mn of 1.00 to 2.00. Mw/Mn is preferably greater than 1.30, and the lower limit may be 1.40, 1.50, or 1.60, and the upper limit may be 1.70, 1.80, or 1.90.

Examples of methods for synthesizing the carboxy-group-containing polymer include a method of polymerizing a monomer to give a repeating unit described above by heating in an organic solvent in the presence of a radical polymerization initiator.

Specific examples of the organic solvent to be used in the polymerization reaction include toluene, benzene, THF, diethyl ether, dioxane, cyclohexane, cyclopentane, cyclopentanone, cyclohexanone, methyl ethyl ketone (MEK), propylene glycol monomethyl ether (PGME), propylene glycol monomethyl ether acetate (PGMEA), and γ-butyrolactone (GBL). Specific examples of the radical polymerization initiator include 2,2′-azobisisobutyronitrile (AIBN), 2,2′-azobis(2,4-dimethylvaleronitrile), dimethyl-2,2′-azobis(2-methylpropionate), 1,1′-azobis(1-acetoxy-1-phenylethane), benzoyl peroxide, and lauroyl peroxide. The amount of the radical polymerization initiator to be added is preferably 0.01 to 25 mol % of the total amount of the monomers to be polymerized. The reaction temperature is preferably 50 to 150° C., more preferably 60 to 100° C. The reaction time is preferably 2 to 24 hours, and from the viewpoint of production efficiency, more preferably 2 to 12 hours.

The radical polymerization initiator may be added to the solution of the monomer and supplied to the reaction vessel, or a solution of the initiator may be prepared separately from the solution of the monomer, and each may be supplied to the reaction vessel independently. There is a possibility that the polymerization reaction may progress due to radicals generated from the initiator during waiting time and an ultra-high molecular weight polymer may be generated, and therefore, from the viewpoint of quality control, it is preferable to prepare each of the monomer solution and the initiator solution independently and add the solutions dropwise. Furthermore, to adjust the molecular weight, a known chain transfer agent, such as dodecyl mercaptan and 2-mercaptoethanol, may also be used. In this case, the amount of the chain transfer agent to be added is preferably 0.01 to 20 mol % of the total amount of the monomers to be polymerized.

Note that the amount of each monomer in the monomer solution can be, for example, set appropriately to achieve the preferable content ratios of the above-described repeating units.

The inventive resist composition may further contain, as optional components, other hypervalent iodine compounds other than the hypervalent iodine compound represented by the general formula (1). When other hypervalent iodine compounds are contained, reactivity to light can be controlled, and sensitivity can be adjusted. As the other hypervalent iodine compounds, hypervalent iodine compounds represented by the following general formula (4) or (5) (hereinafter, also referred to as other hypervalent iodine compounds) are preferable.

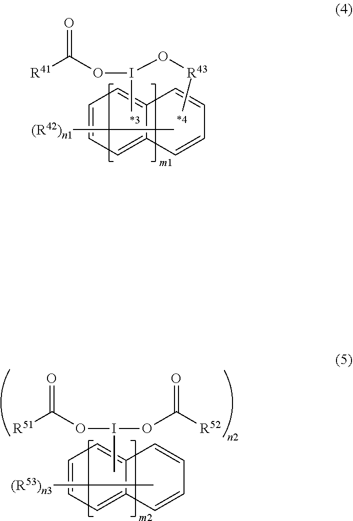

In the formulae, “m1” and “m2” each represent an integer of 0 to 2.

“n1” represents an integer of 0 to 4 when “m1” is 0, an integer of 0 to 6 when “m1” is 1, and an integer of 0 to 8 when “m1” is 2.

When “m2” is 0, “n2” represents an integer of 1 to 3, “n3” represents an integer of 0 to 5, and 1≤(n2+n3)≤6 is satisfied.

When “m2” is 1, “n2” represents an integer of 1 to 3, “n3” represents an integer of 0 to 7, and 1≤(n2+n3)≤8 is satisfied.

When “m2” is 2, “n2” represents an integer of 1 to 3, “n3” represents an integer of 0 to 9, and 1≤(n2+n3)≤10 is satisfied.

R⁴¹ represents a halogen atom or a hydrocarbyl group having 1 to 10 carbon atoms and optionally containing a heteroatom.

R⁴² represents a halogen atom or a hydrocarbyl group having 1 to 40 carbon atoms and optionally containing a heteroatom, when “n1” is 2 to 8, the R⁴²'s being identical to or different from each other and the R⁴²'s optionally being bonded to each other to form a ring together with the carbon atoms of the aromatic ring bonded to the R⁴²'s.

R⁴³ represents a carbonyl group or a hydrocarbylene group having 1 to 10 carbon atoms and optionally containing a heteroatom.

“*3” and “*4” each represent an attachment point to one of the carbon atoms of the aromatic ring in the formula. However, “*3” and “*4” need to be bonded to adjacent carbon atoms of the aromatic ring.

R⁵¹ and R⁵² each independently represent a halogen atom or a hydrocarbyl group having 1 to 10 carbon atoms and optionally containing a heteroatom, the R⁵¹ and the R⁵² optionally being bonded to each other to form a ring together with the carbon atoms bonded thereto and the atoms between the carbon atoms.

R⁵³ represents a halogen atom or a hydrocarbyl group having 1 to 40 carbon atoms and optionally containing a heteroatom, when “n3” is 2 to 9, the R⁵³'s being identical to or different from each other and the R⁵³'s optionally being bonded to each other to form a ring together with the carbon atoms of the aromatic ring bonded to the R⁵³'s.

In the general formula (4), “m1” represents an integer of 0 to 2. “n1” represents an integer of 0 to 4 when “m1” is 0, an integer of 0 to 6 when “m1” is 1, and an integer of 0 to 8 when “m1” is 2. “n1” is preferably 0, 1, 2, 3, or 4, more preferably 0, 1, 2, or 3, further preferably 0, 1, or 2, and most preferably 0 or 1.

In the general formula (4), R⁴¹ represents a halogen atom or a hydrocarbyl group having 1 to 10 carbon atoms and optionally containing a heteroatom. Specific examples of the halogen atom include a fluorine atom, a chlorine atom, a bromine atom, and an iodine atom. The hydrocarbyl group having 1 to 10 carbon atoms may be saturated or unsaturated, and may be linear, branched, or cyclic. Specific examples thereof include: alkyl groups having 1 to 10 carbon atoms, such as a methyl group, an ethyl group, an n-propyl group, an isopropyl group, an n-butyl group, an isobutyl group, a sec-butyl group, a tert-butyl group, an n-pentyl group, a tert-pentyl group, an n-hexyl group, an n-octyl group, a 2-ethylhexyl group, an n-nonyl group, and an n-decyl group; cyclic saturated hydrocarbyl groups having 3 to 10 carbon atoms, such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, a tricyclo[5.2.1.0^{2,6}]decanyl group, and an adamantyl group; alkenyl groups having 2 to 10 carbon atoms, such as a vinyl group and an allyl group; aryl groups having 6 to 10 carbon atoms, such as a phenyl group and a naphthyl group; and groups which are combinations of these groups. Furthermore, part or all of the hydrogen atoms of the hydrocarbyl group may be substituted with a group containing a heteroatom, such as an oxygen atom, a sulfur atom, a nitrogen atom, or a halogen atom, and part of the —CH₂— of the hydrocarbyl group may be substituted with a group containing a heteroatom such as an oxygen atom, a sulfur atom, or a nitrogen atom. The resulting hydrocarbyl group may contain a hydroxy group, a cyano group, a halogen atom, a carbonyl group, an ether bond, a thioether bond, an ester bond, a sulfonic acid ester bond, a carbonate bond, a carbamate bond, a lactone ring, a sultone ring, a carboxylic anhydride (—C(═O)—O—C(═O)—), etc. As the R⁴¹, a hydrocarbyl group having 1 to 4 carbon atoms or a fluorinated hydrocarbyl group having 1 to 4 carbon atoms is preferable, and a hydrocarbyl group having 1 to 4 carbon atoms is more preferable.

In the general formula (4), R⁴² represents a halogen atom or a hydrocarbyl group having 1 to 40 carbon atoms and optionally containing a heteroatom. Specific examples of the halogen atom include a fluorine atom, a chlorine atom, a bromine atom, and an iodine atom. The hydrocarbyl group having 1 to 40 carbon atoms may be saturated or unsaturated, and may be linear, branched, or cyclic. Specific examples thereof include: alkyl groups having 1 to 40 carbon atoms, such as a methyl group, an ethyl group, an n-propyl group, an isopropyl group, an n-butyl group, an isobutyl group, a sec-butyl group, a tert-butyl group, an n-pentyl group, a tert-pentyl group, an n-hexyl group, an n-octyl group, a 2-ethylhexyl group, an n-nonyl group, and an n-decyl group; cyclic saturated hydrocarbyl groups having 3 to 40 carbon atoms, such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, a tricyclo[5.2.1.0^{2,6}]decanyl group, an adamantyl group, and an adamantylmethyl group; and aryl groups having 6 to 40 carbon atoms, such as a phenyl group, a naphthyl group, and an anthracenyl group. Furthermore, part or all of the hydrogen atoms of the hydrocarbyl group may be substituted with a group containing a heteroatom, such as an oxygen atom, a sulfur atom, a nitrogen atom, or a halogen atom, and part of the —CH₂— of the hydrocarbyl group may be substituted with a group containing a heteroatom such as an oxygen atom, a sulfur atom, or a nitrogen atom. The resulting hydrocarbyl group may contain a hydroxy group, a cyano group, a halogen atom, a carbonyl group, an ether bond, a thioether bond, an ester bond, a sulfonic acid ester bond, a carbonate bond, a carbamate bond, a lactone ring, a sultone ring, a carboxylic anhydride (—C(═O)—O—C(═O)—), etc. When “n1” is 2 to 8, the R⁴²'s are identical to or different from each other and the R⁴²'s are optionally bonded to each other to form a ring together with the carbon atoms of the aromatic ring bonded to the R⁴²'s.

In the general formula (4), R⁴³ represents a carbonyl group or a hydrocarbylene group having 1 to 10 carbon atoms and optionally containing a heteroatom. The hydrocarbylene group having 1 to 10 carbon atoms may be saturated or unsaturated, and may be linear, branched, or cyclic. Specific examples thereof include: alkylene groups having 1 to 10 carbon atoms, such as a methanediyl group, an ethane-1,1-diyl group, an ethane-1,2-diyl group, a propane-1,1-diyl group, a propane-1,2-diyl group, a propane-1,3-diyl group, a propane-2,2-diyl group, a butane-2,3-diyl group, a butane-1,4-diyl group, a 2-methylpropane-1,2-diyl group, a pentane-1,5-diyl group, a hexane-1,6-diyl group, a heptane-1,7-diyl group, an octane-1,8-diyl group, a nonane-1,9-diyl group, and a decane-1,10-diyl group; cyclic saturated hydrocarbylene groups having 3 to 10 carbon atoms, such as a cyclopentanediyl group, a cyclohexanediyl group, a norbornanediyl group, an adamantanediyl group, and a tricyclo[5.2.1.0^{2,6}]decanediyl group; alkenylene groups having 2 to 10 carbon atoms, such as a vinylene group and a propynylene group; arylene groups having 6 to 10 carbon atoms, such as a phenylene group, a methylphenylene group, an ethylphenylene group, an n-propylphenylene group, an isopropylphenylene group, an n-butylphenylene group, and a naphthylene group; and groups which are combinations of these groups. Furthermore, part or all of the hydrogen atoms of the hydrocarbylene group may be substituted with a group containing a heteroatom, such as an oxygen atom, a sulfur atom, a nitrogen atom, or a halogen atom, and part of the —CH₂— of the hydrocarbylene group may be substituted with a group containing a heteroatom such as an oxygen atom, a sulfur atom, or a nitrogen atom. The resulting hydrocarbylene group may contain a hydroxy group, a cyano group, a halogenated alkyl group, a halogen atom, a carbonyl group, an ether bond, a thioether bond, an ester bond, a sulfonic acid ester bond, a carbonate bond, a carbamate bond, a lactone ring, a sultone ring, a carboxylic anhydride (—C(═O)—O—C(═O)—), etc. As the R⁴³, a carbonyl group, a hydrocarbylene group having 1 to 4 carbon atoms, or a fluorinated hydrocarbylene group having 1 to 4 carbon atoms is preferable.

In the general formula (4), “*3” and “*4” each represent an attachment point to one of the carbon atoms of the aromatic ring in the formula, provided that “*3” and “*4” are bonded to adjacent carbon atoms of the aromatic ring. As combinations of such “*3”, “*4”, and “m1”, the seven cases shown below are possible.

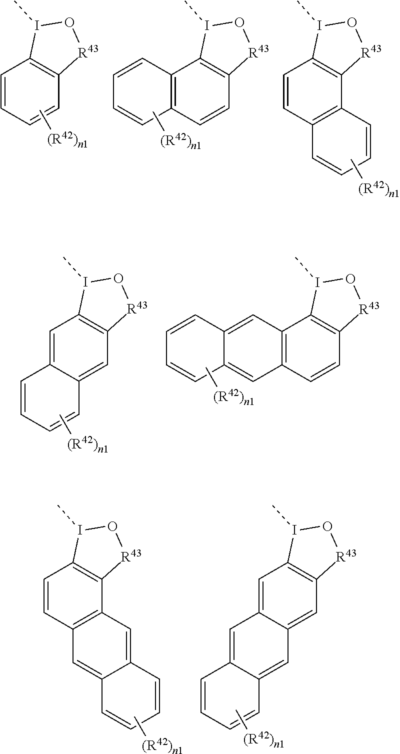

In the formulae, “n1”, R⁴², and R⁴³ are as defined above. A broken line represents an attachment point to R⁴¹—C(═O)—O—.

Specific examples of the hypervalent iodine compound represented by the general formula (4) include the following, but are not limited thereto. Note that, in the following formulae, Me represents a methyl group.

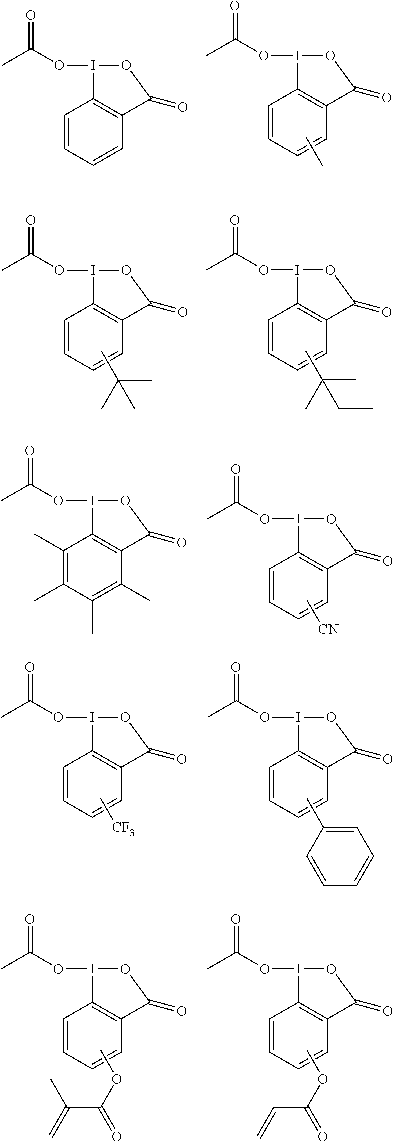

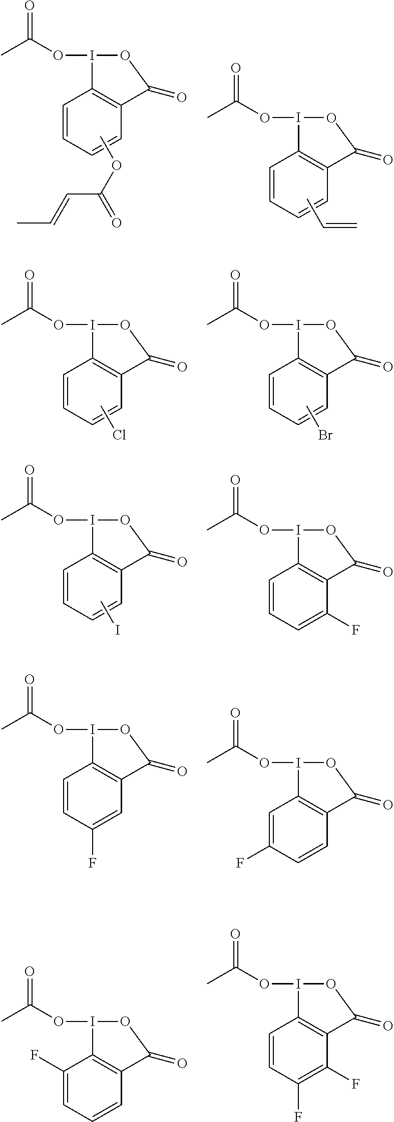

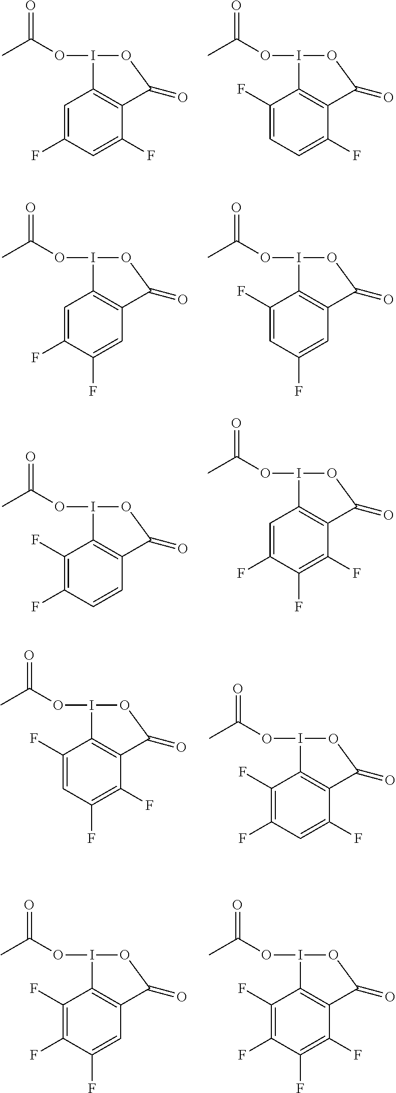

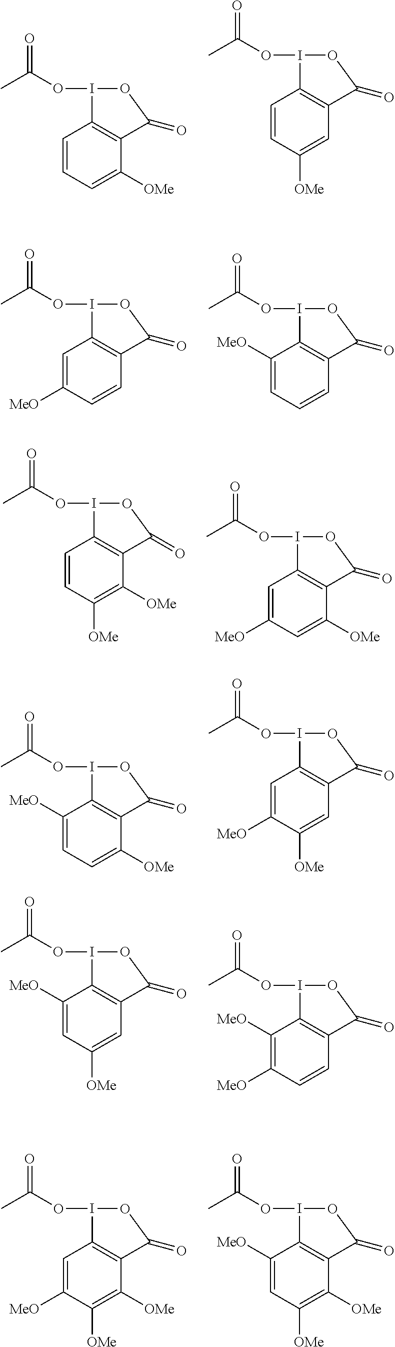

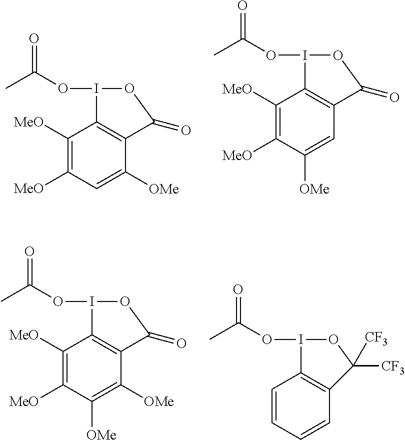

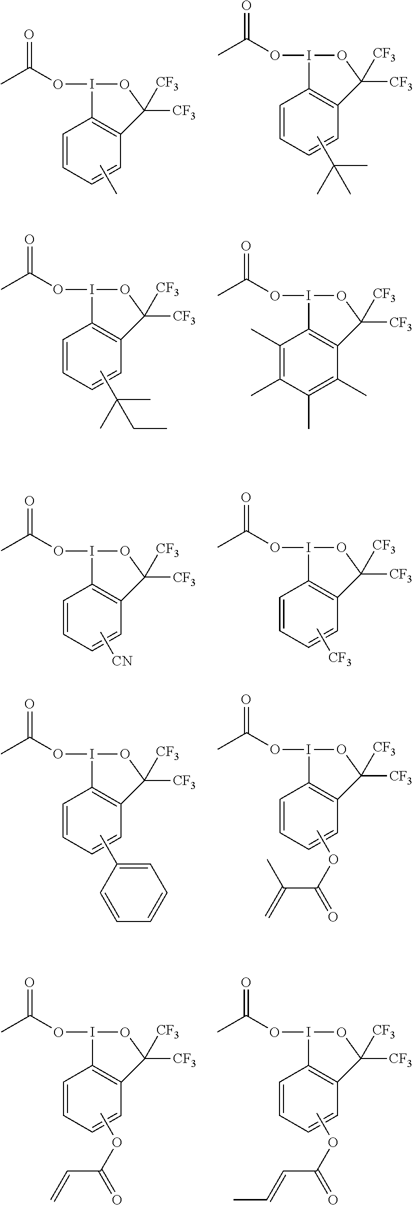

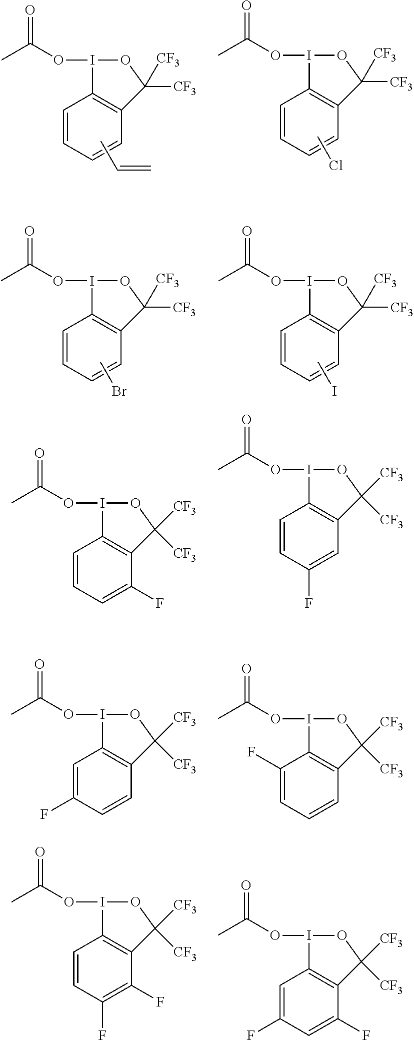

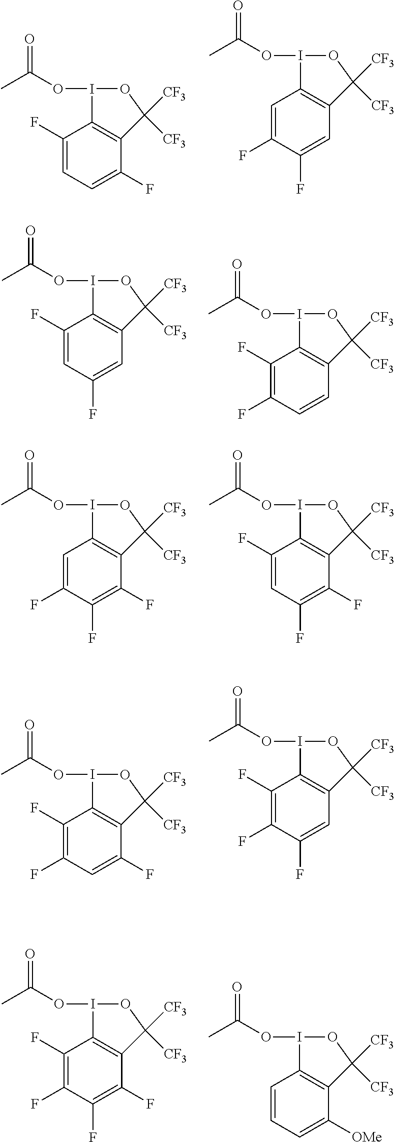

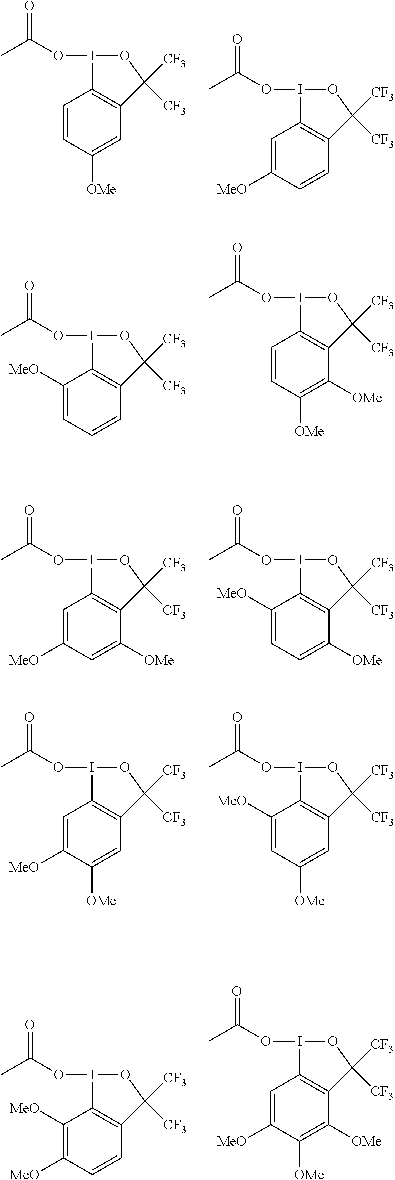

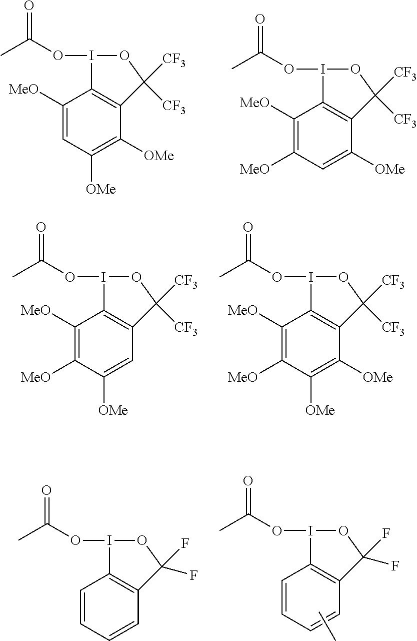

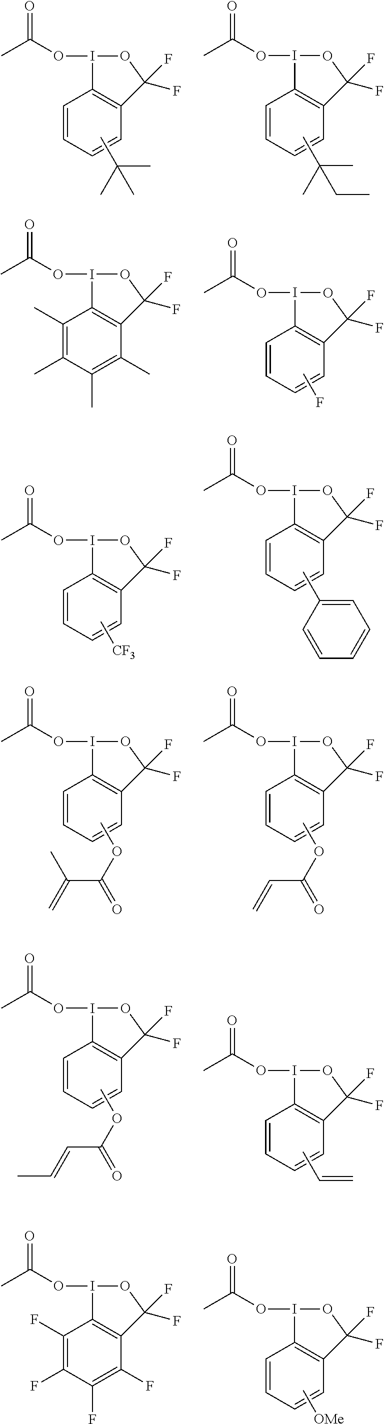

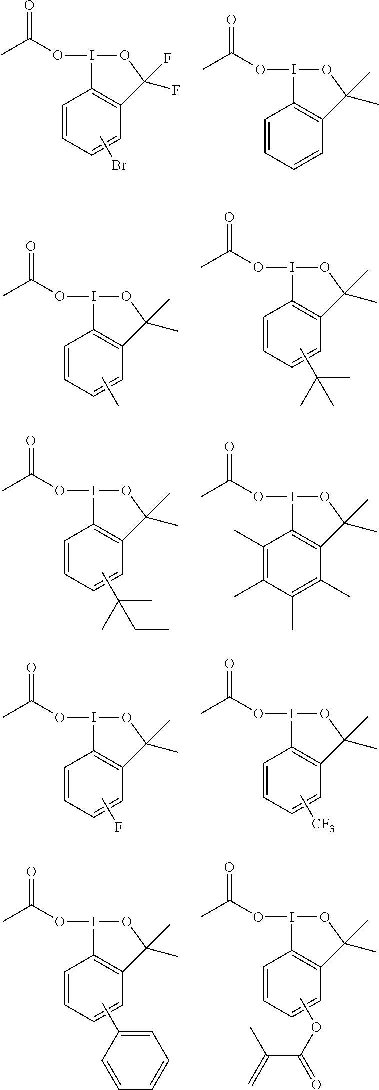

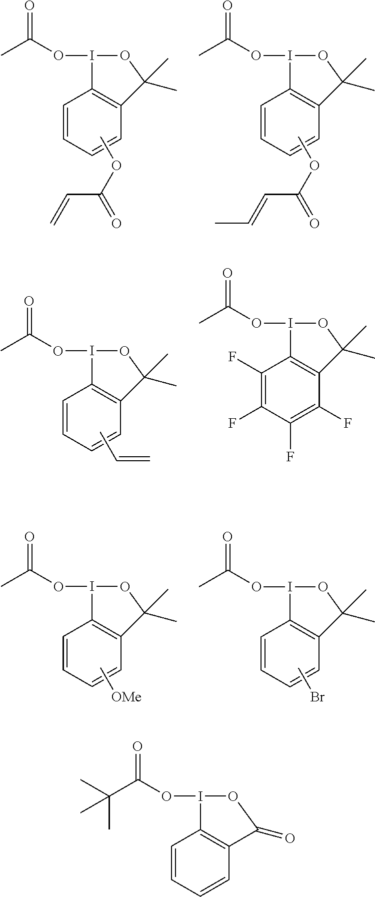

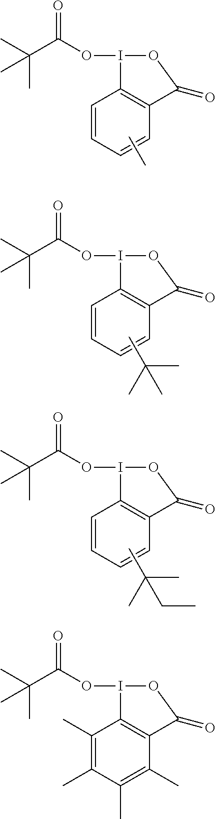

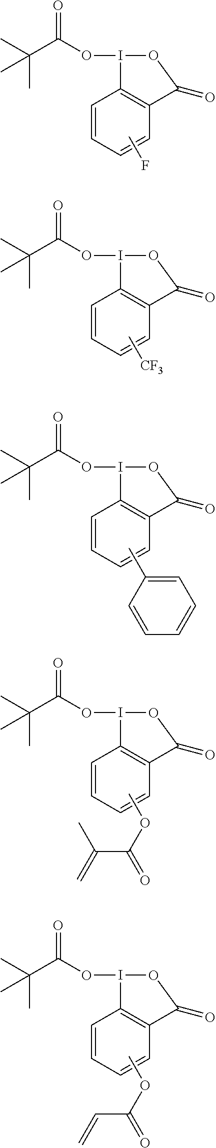

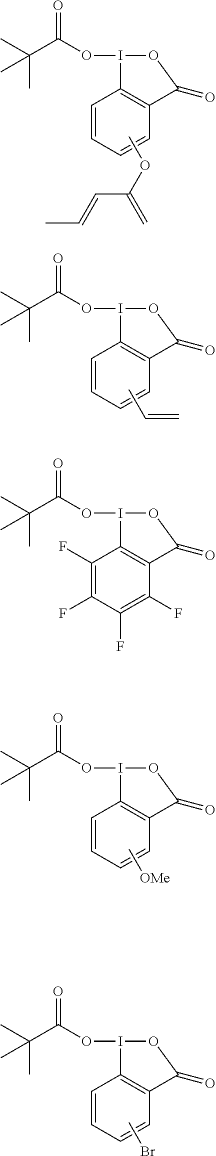

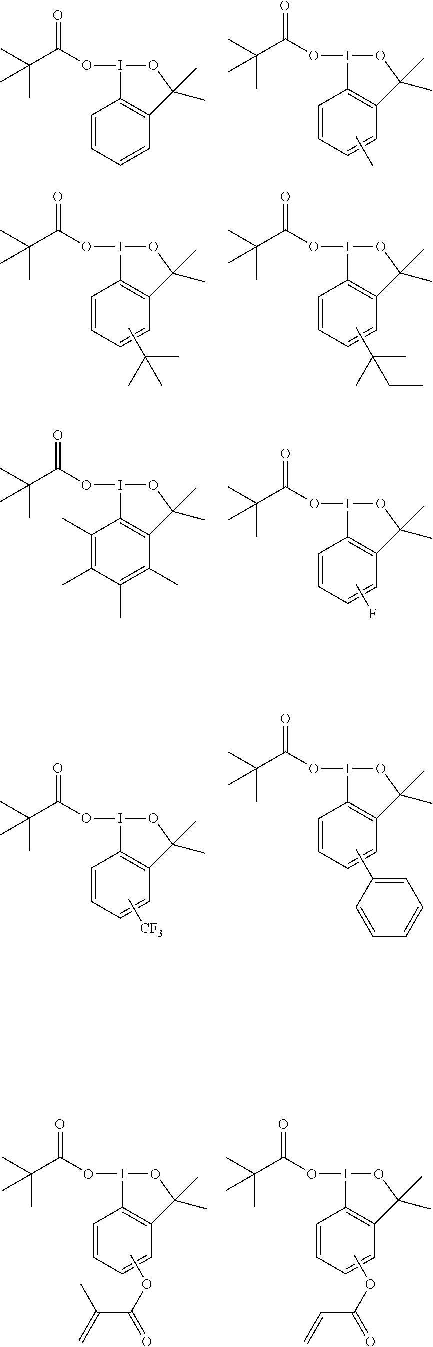

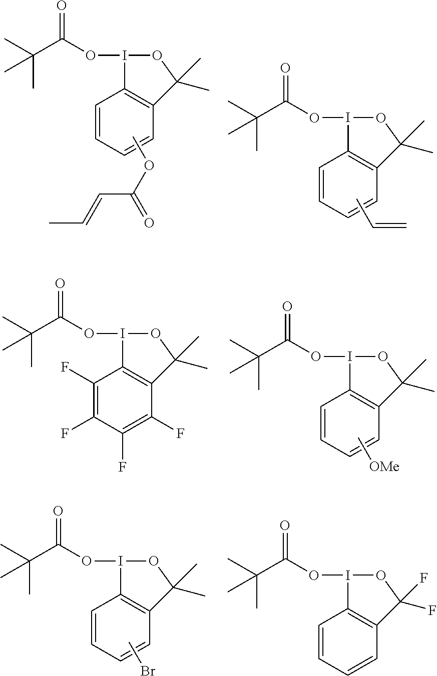

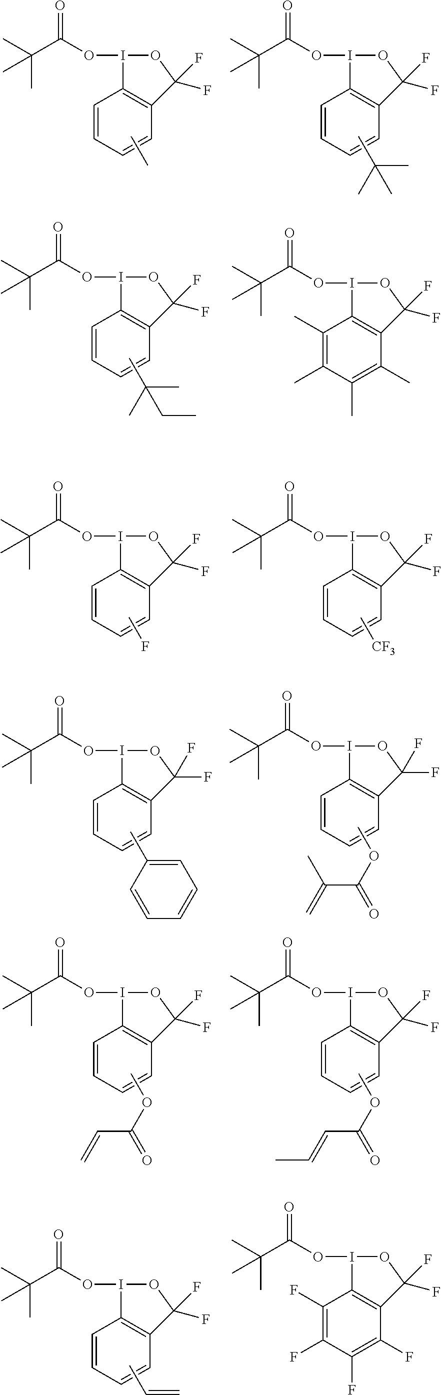

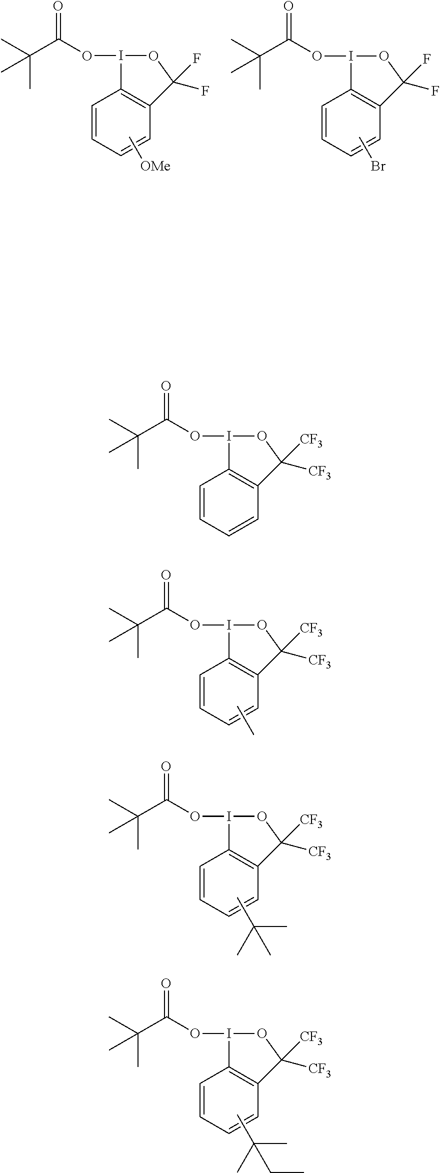

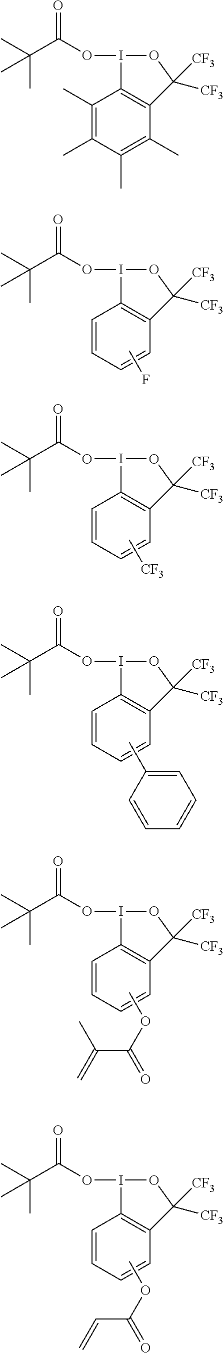

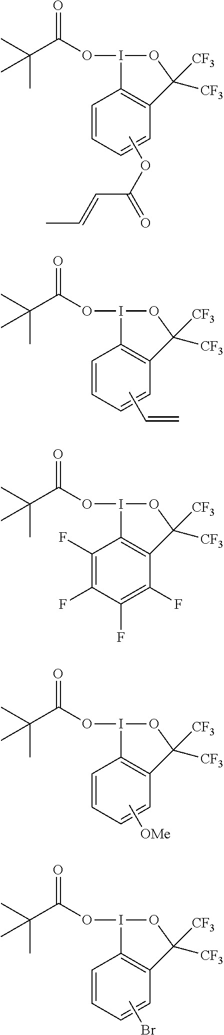

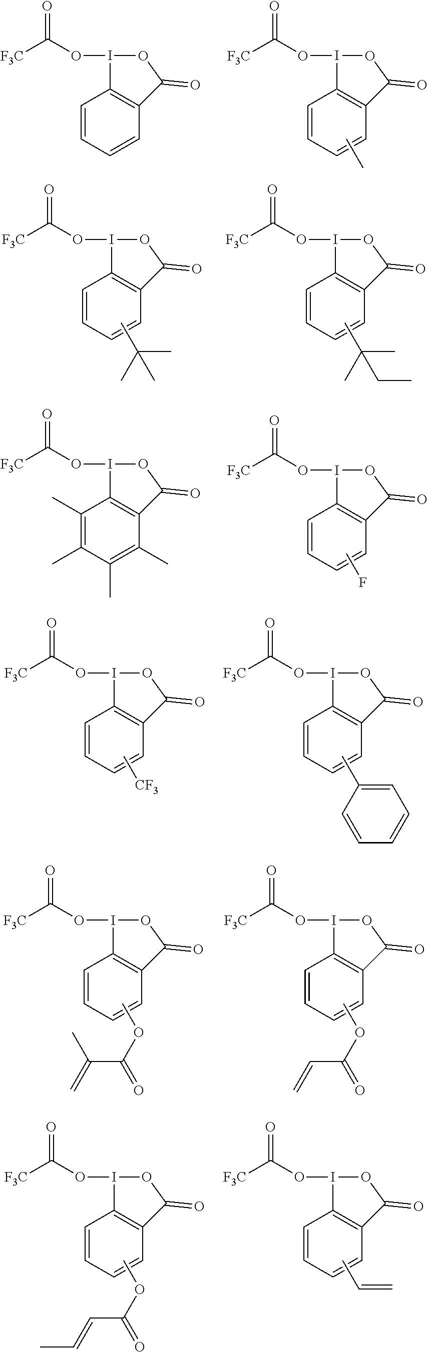

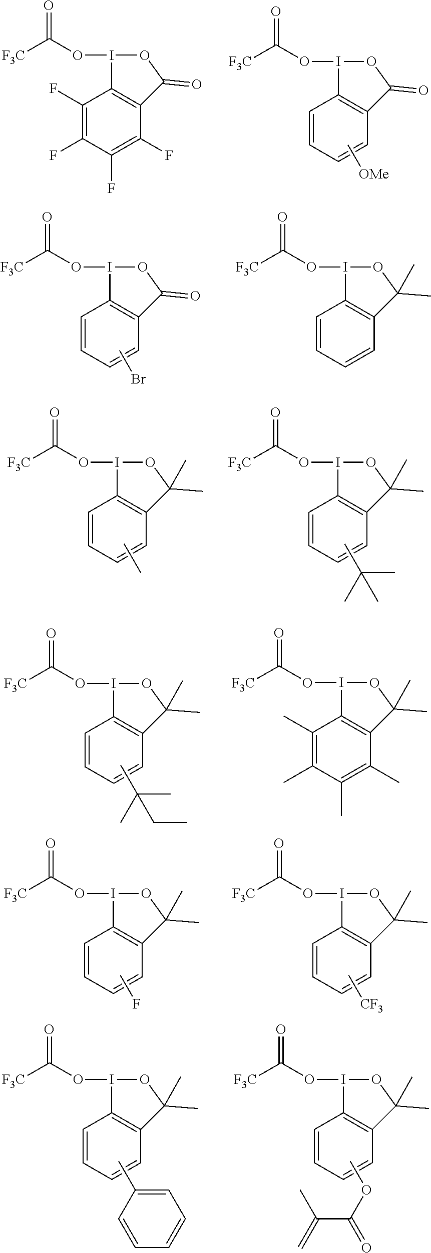

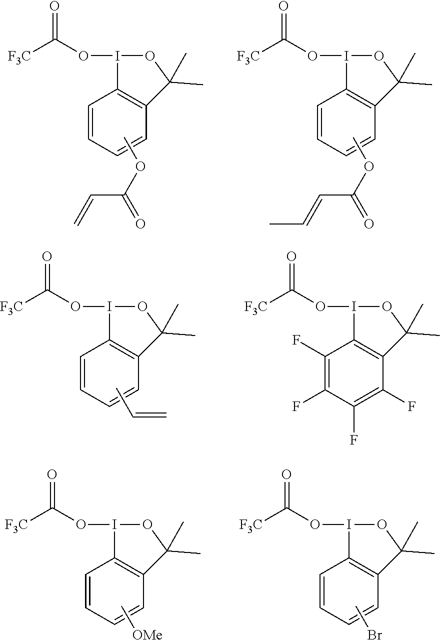

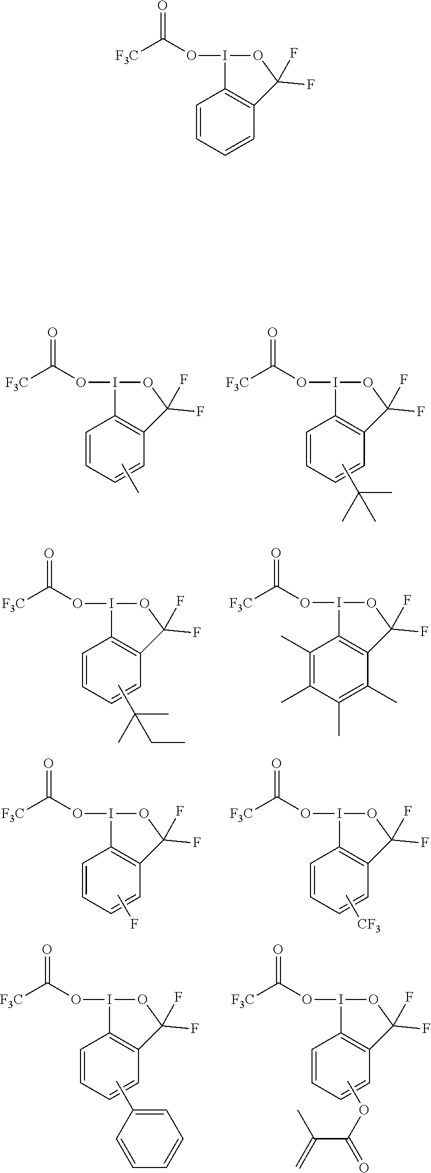

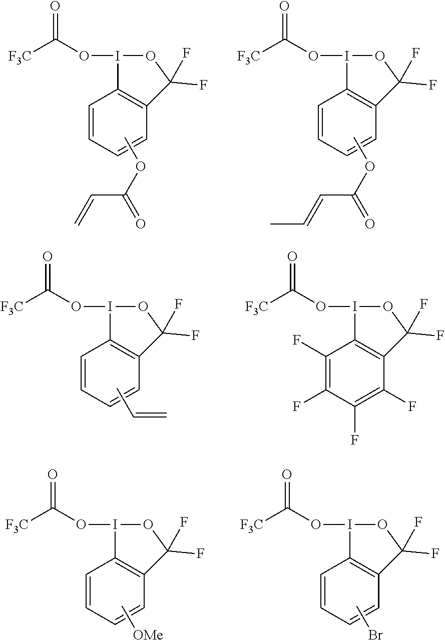

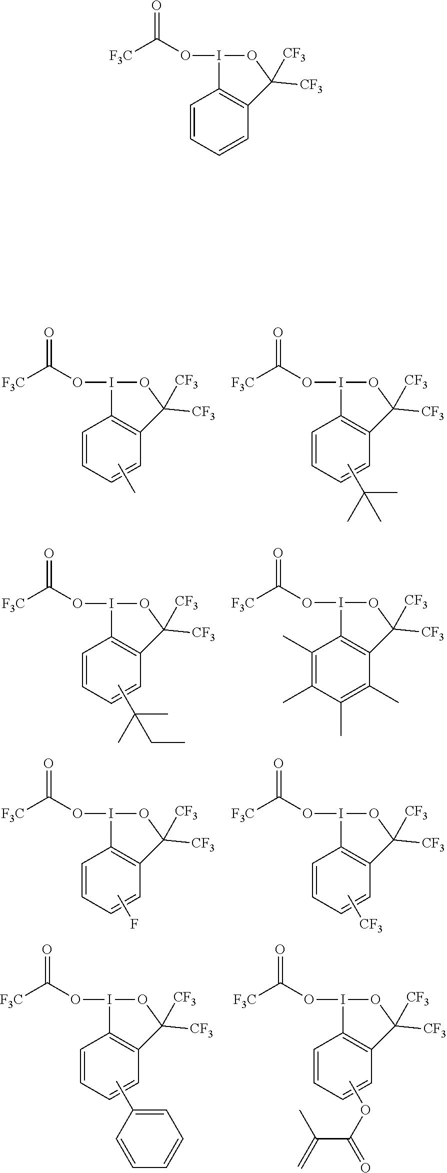

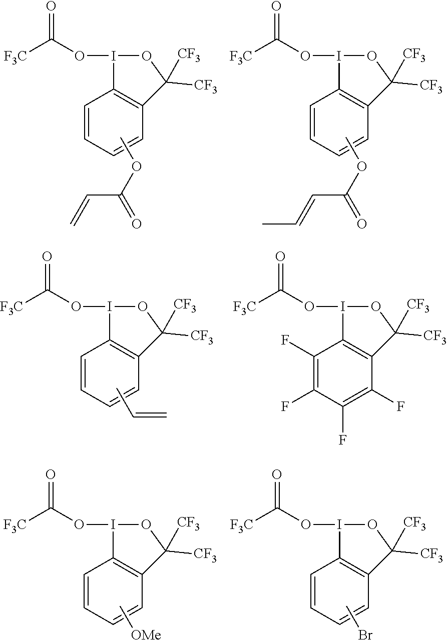

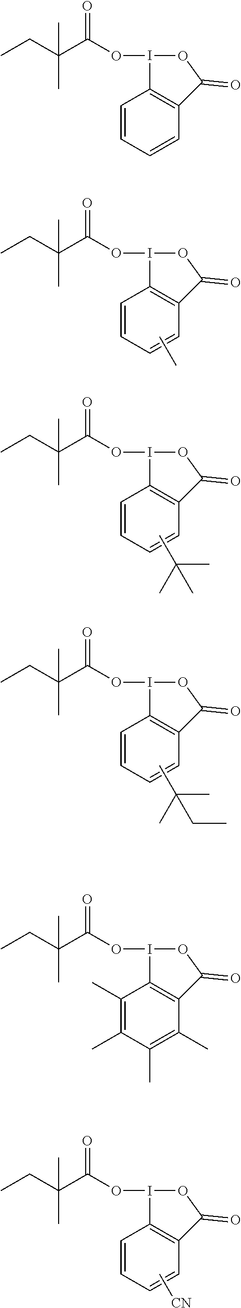

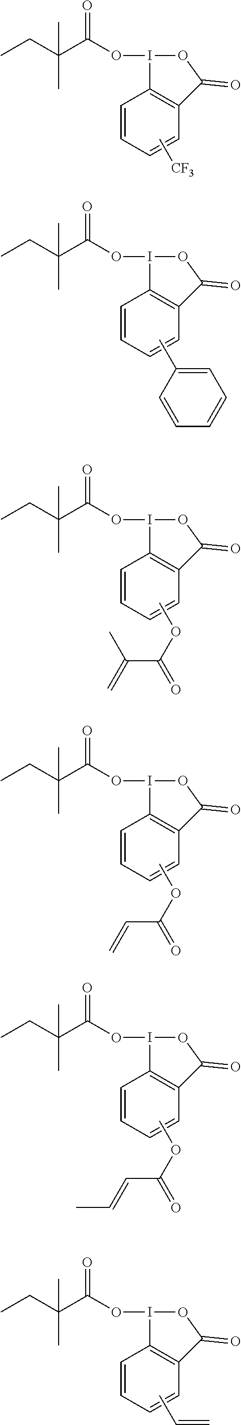

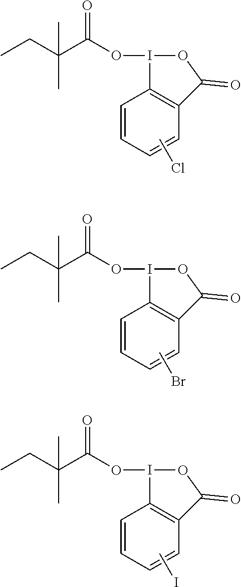

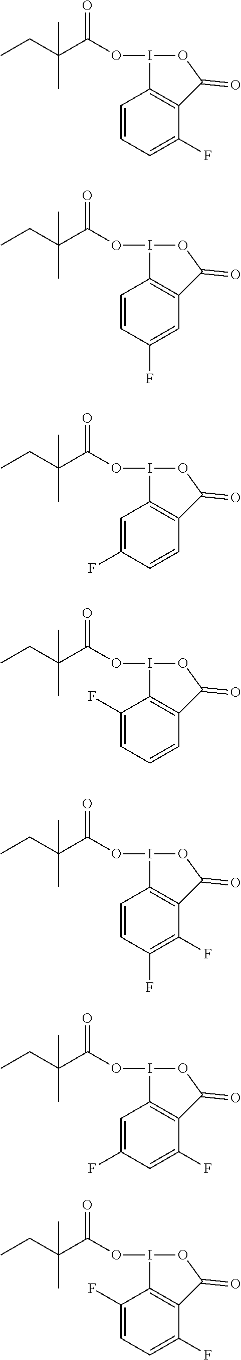

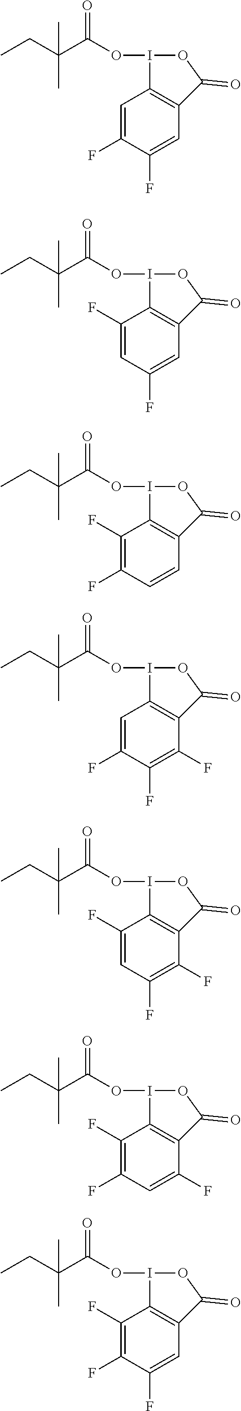

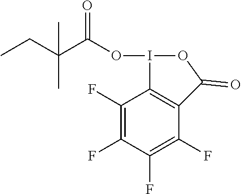

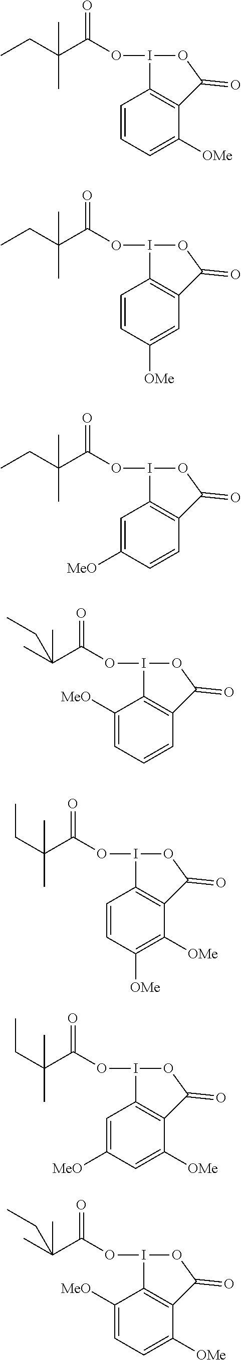

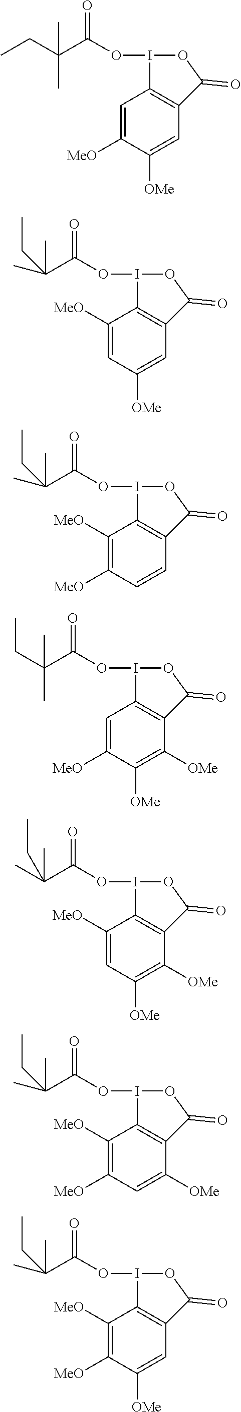

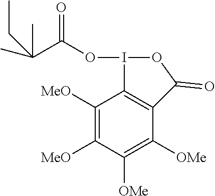

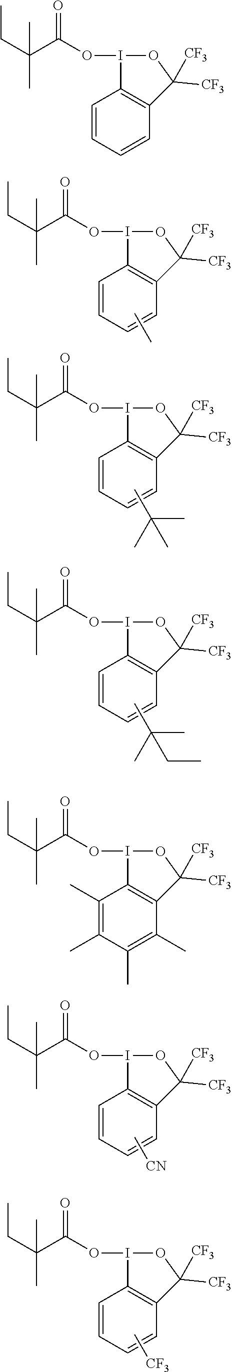

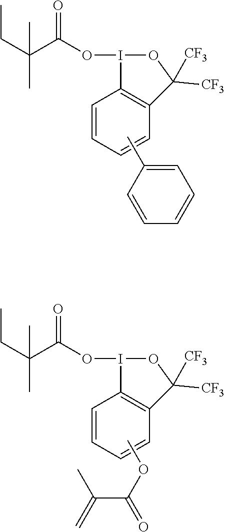

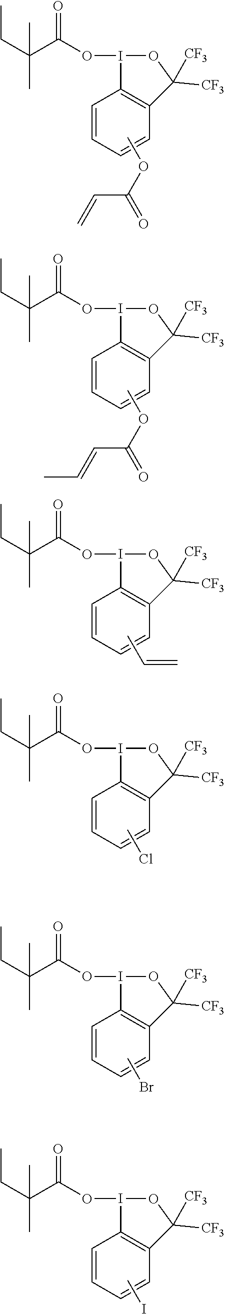

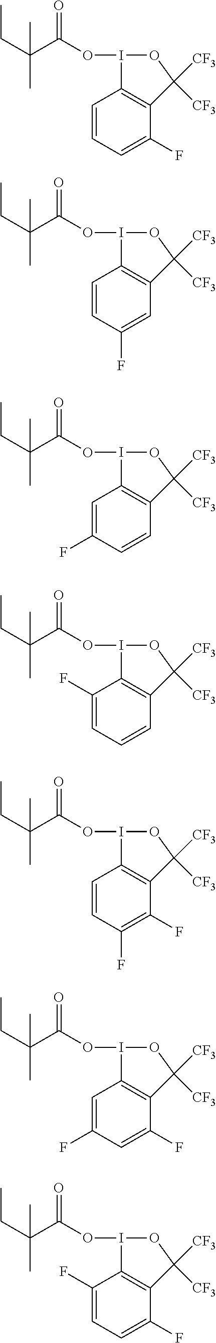

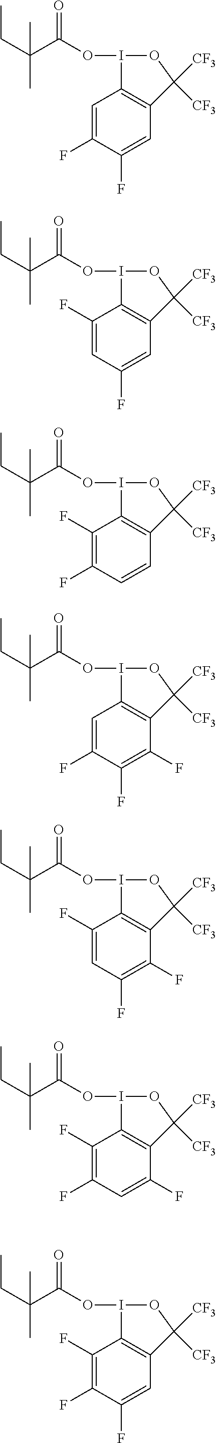

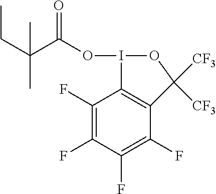

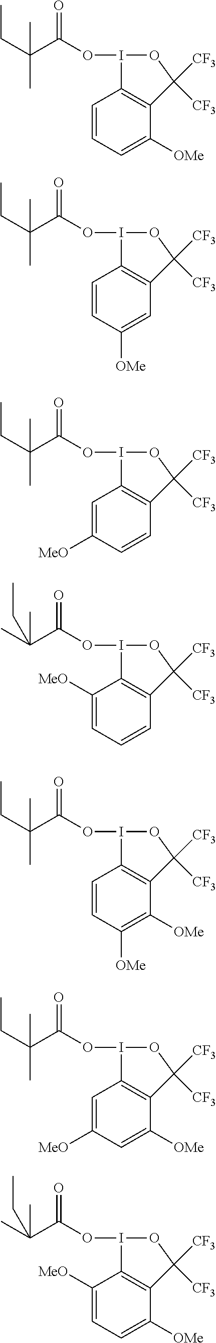

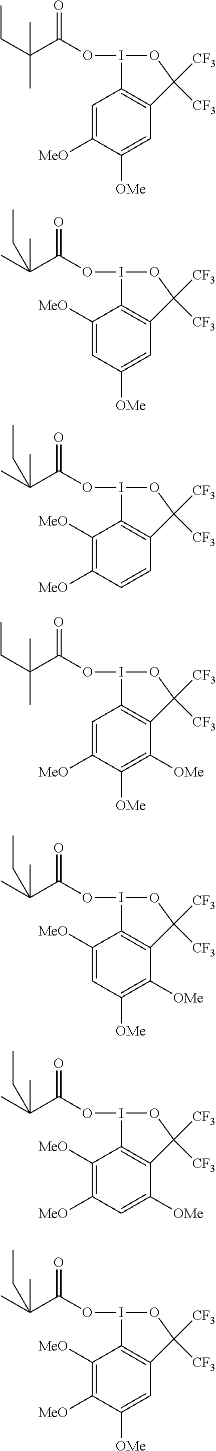

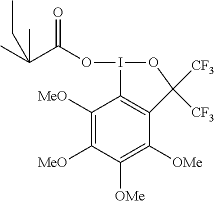

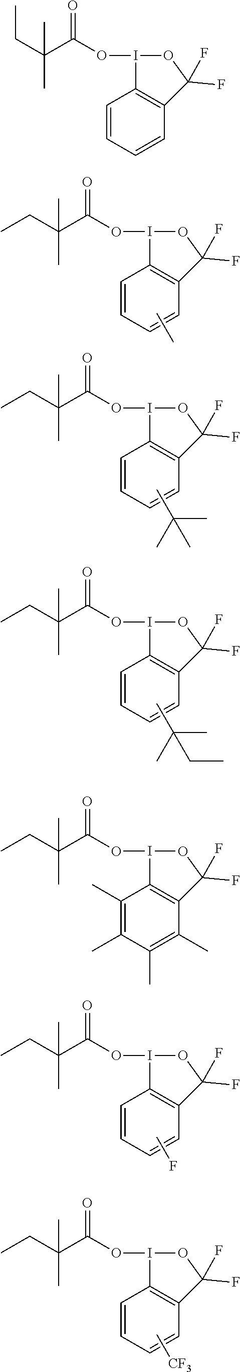

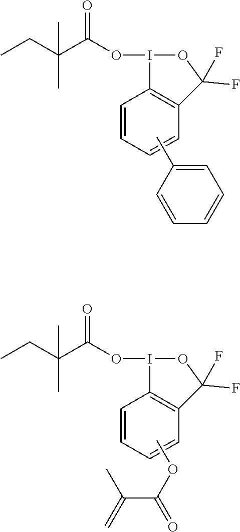

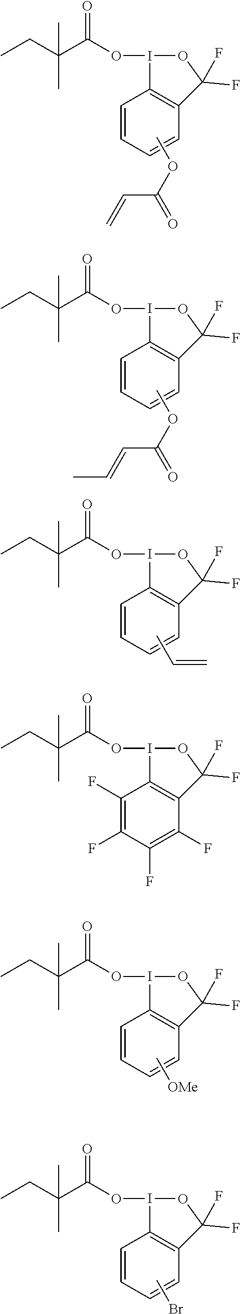

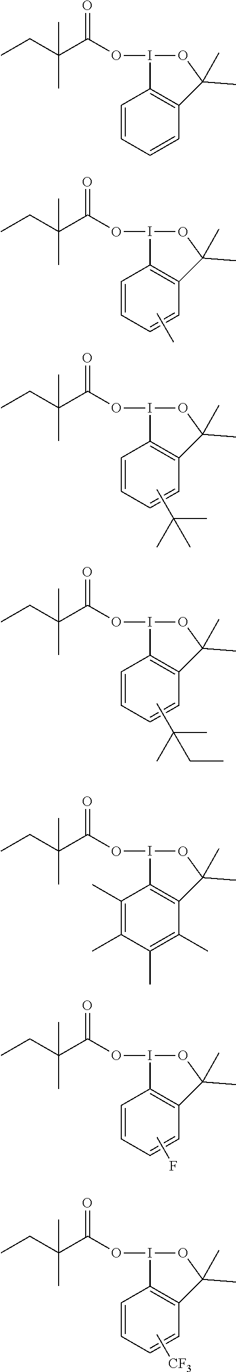

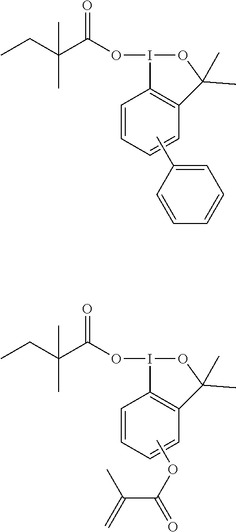

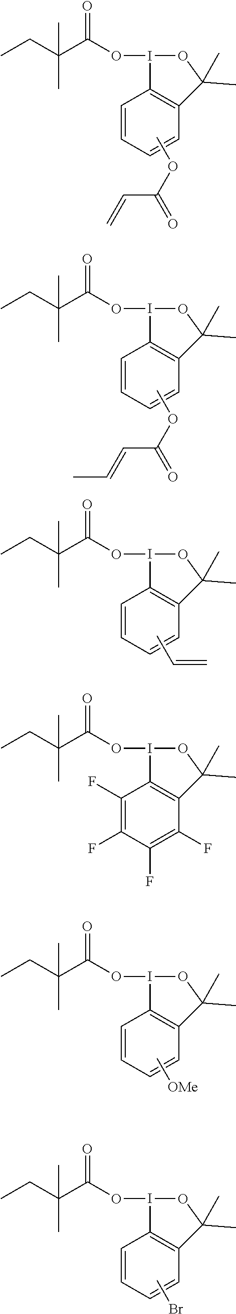

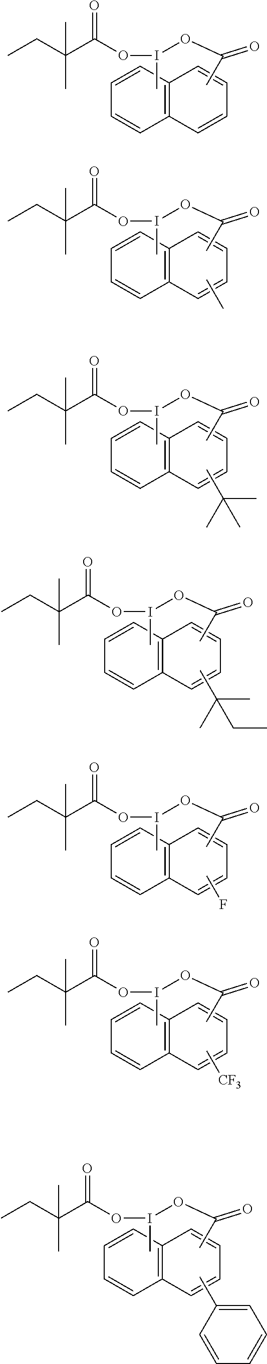

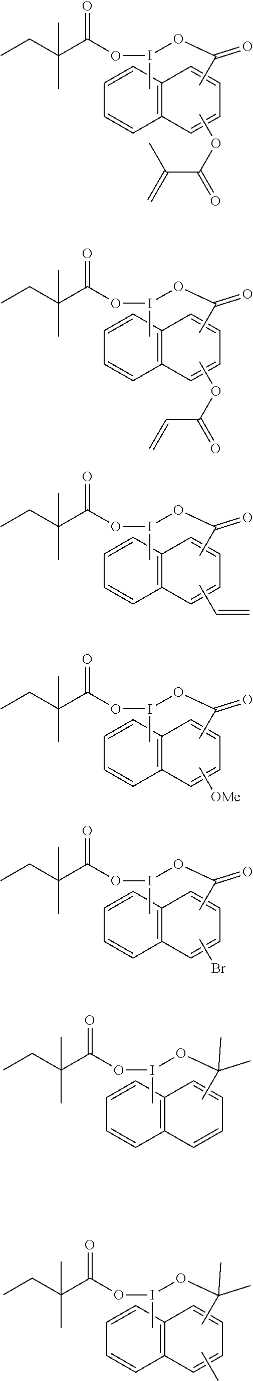

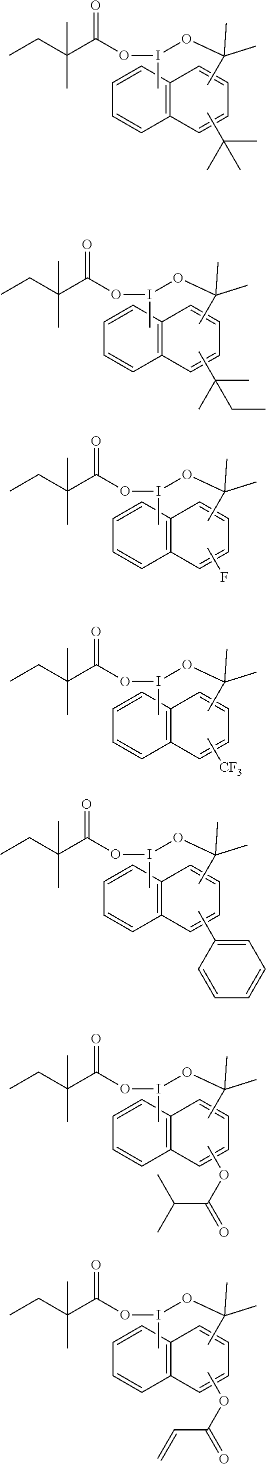

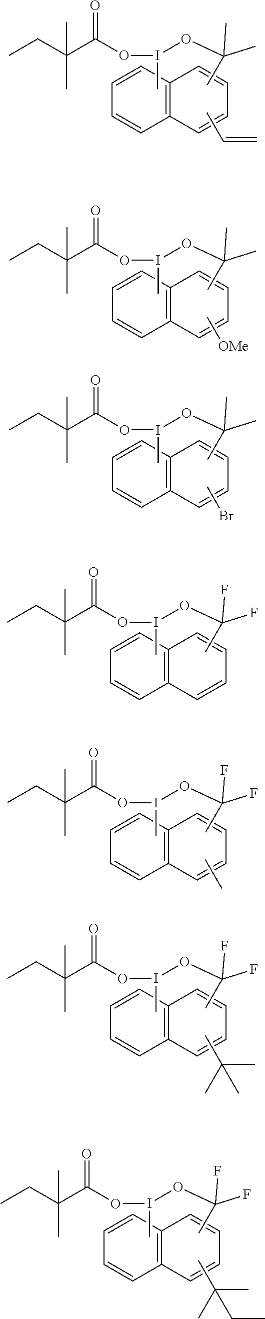

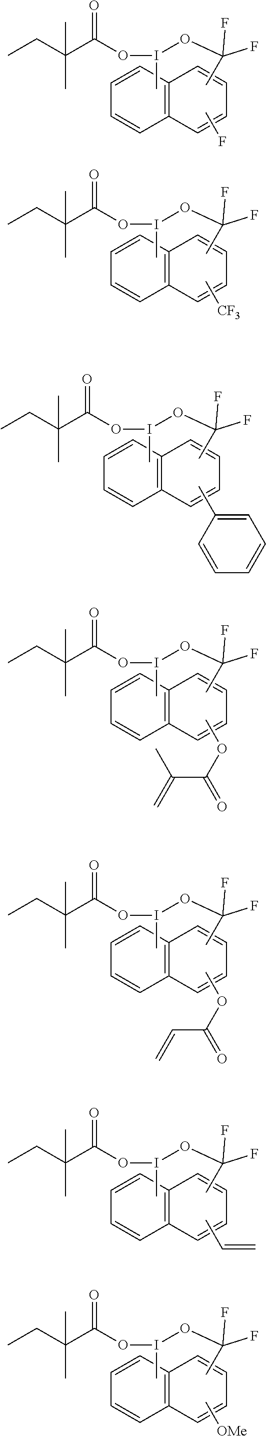

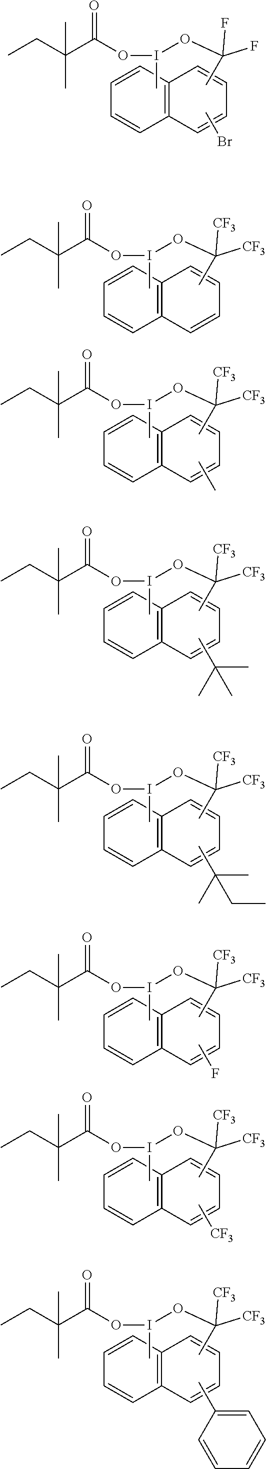

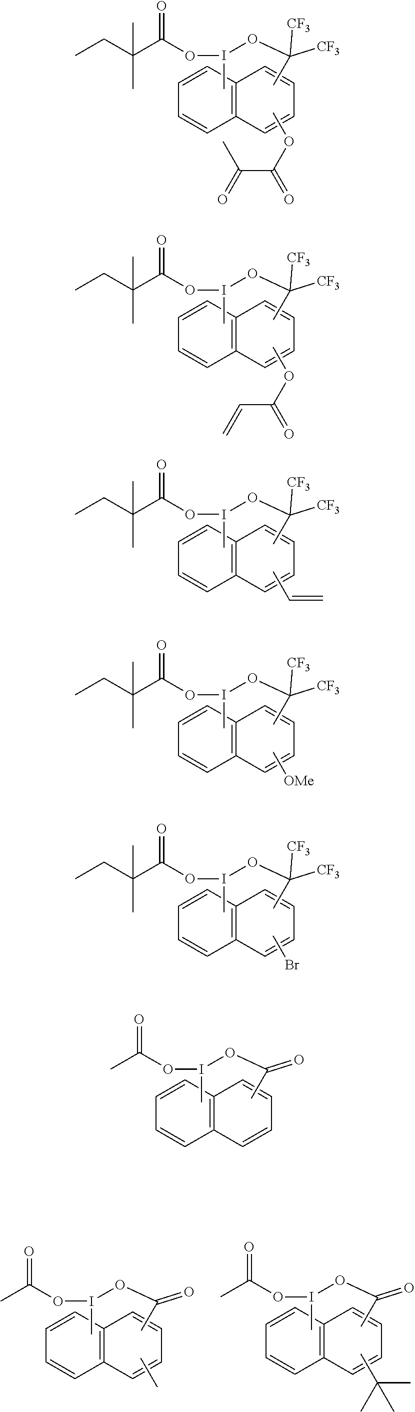

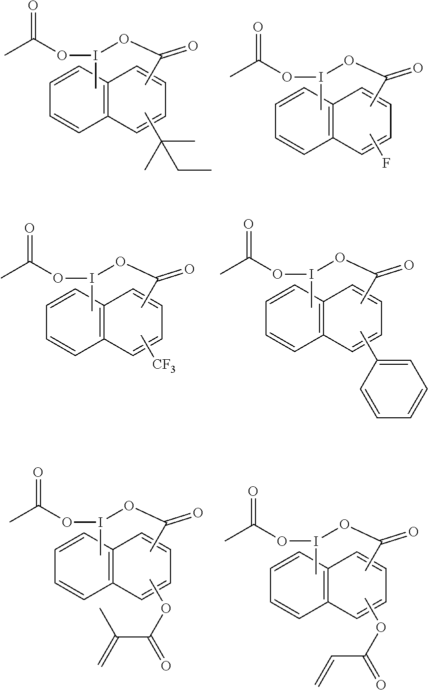

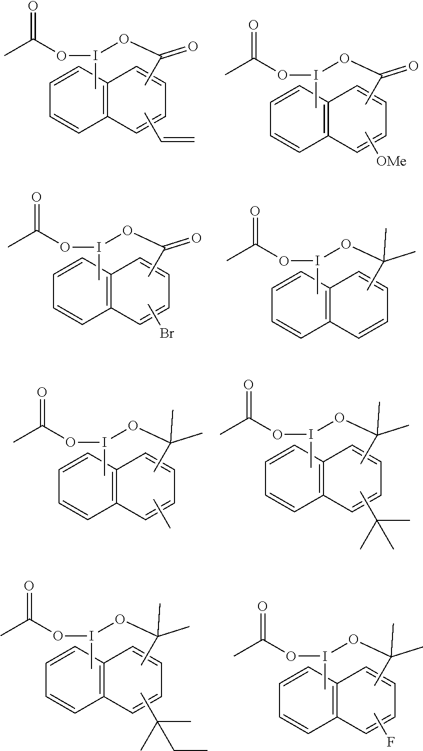

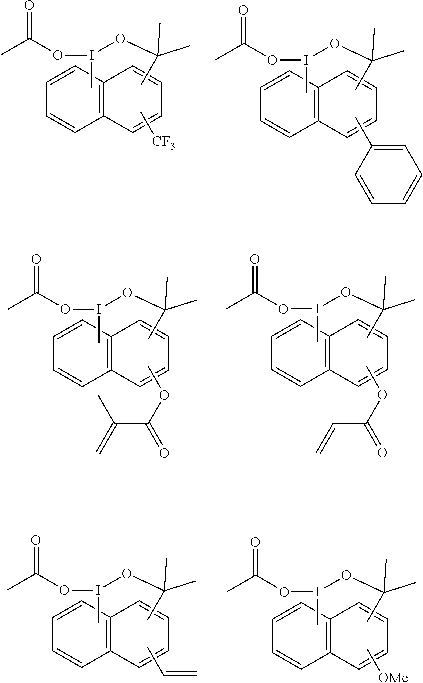

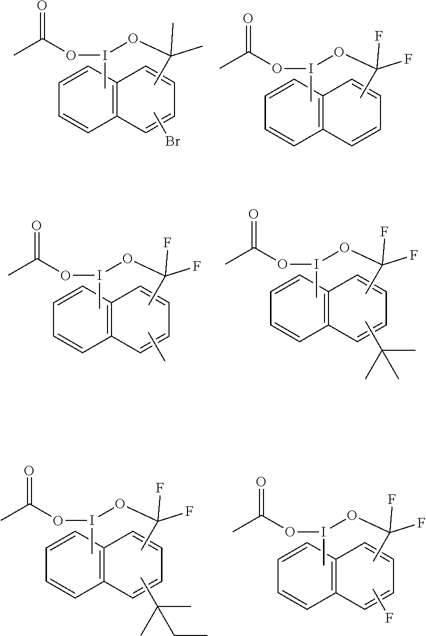

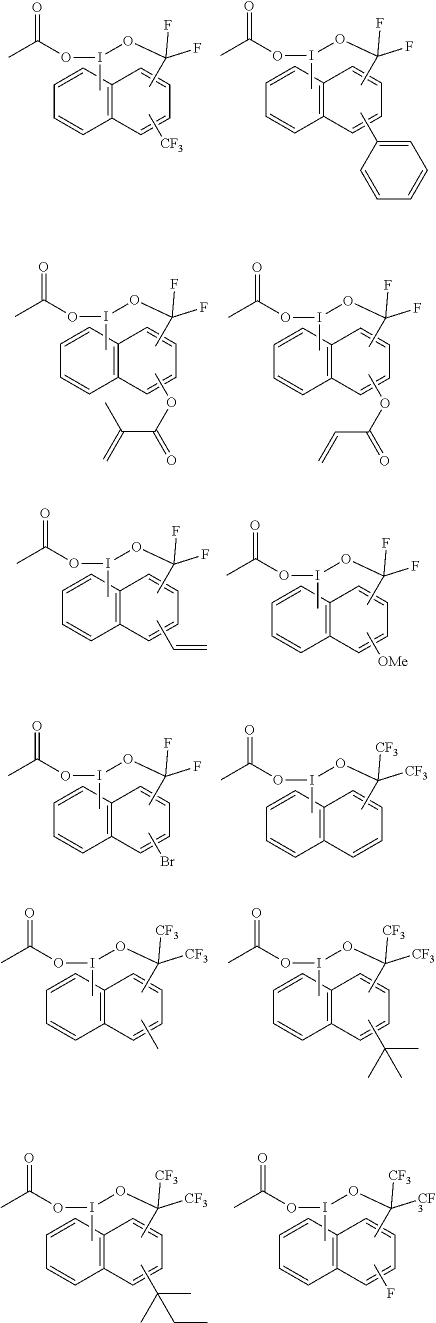

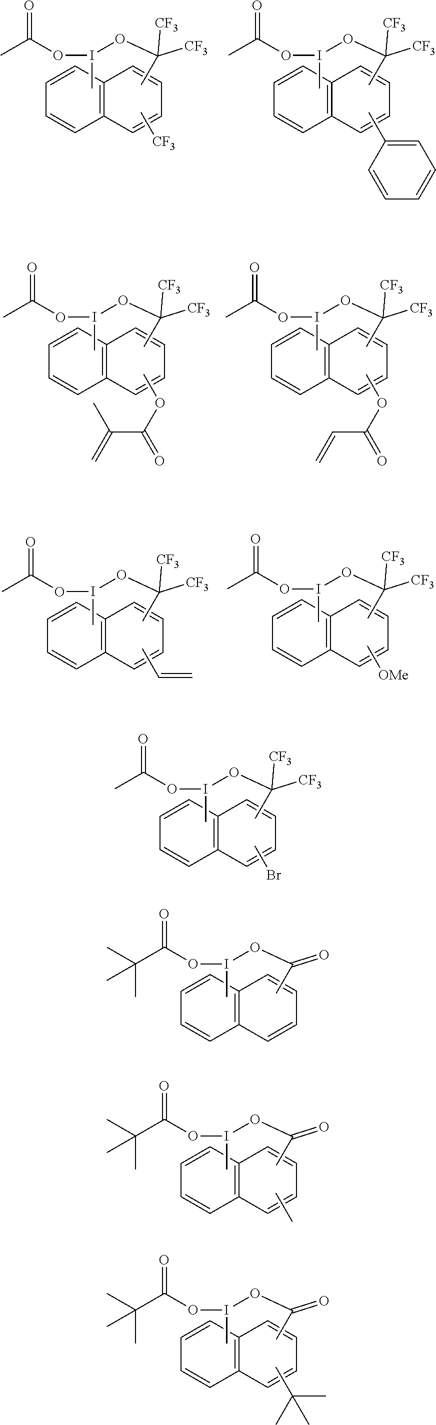

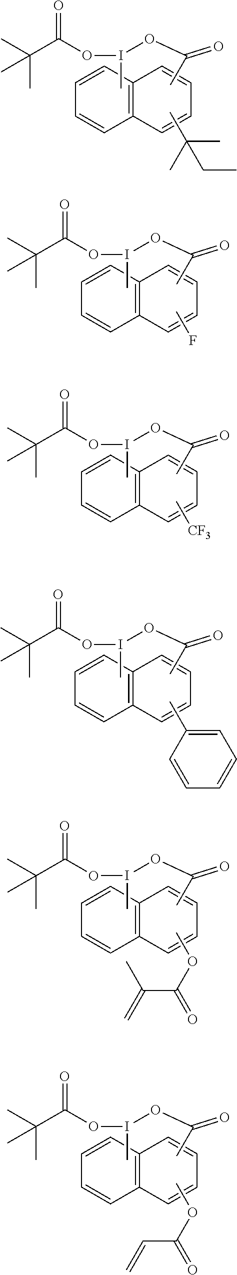

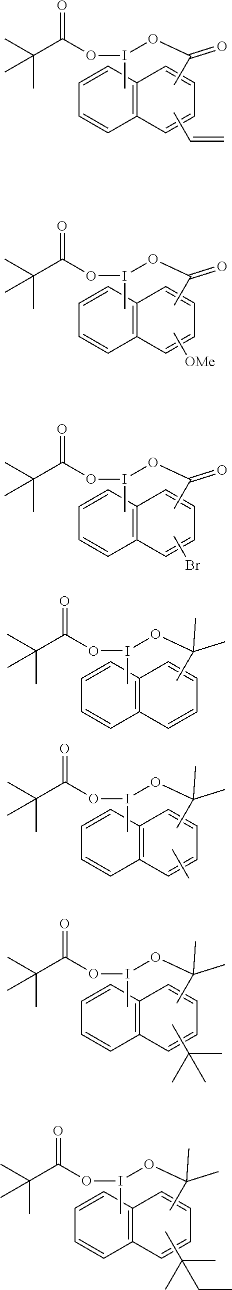

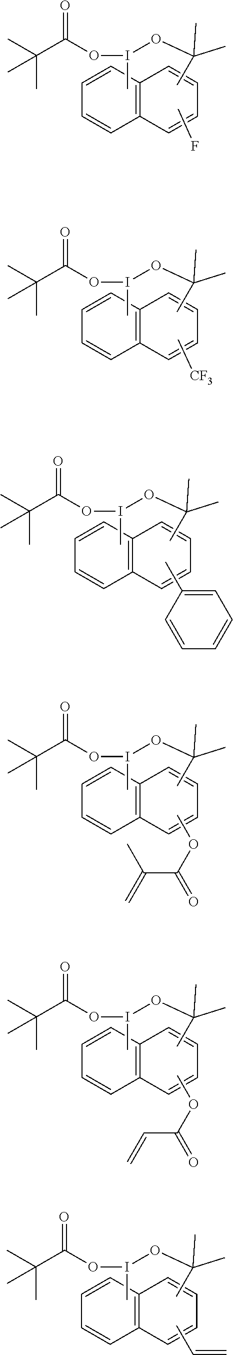

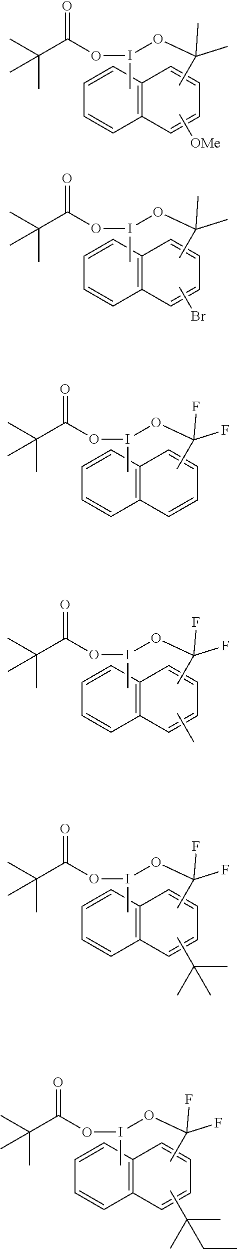

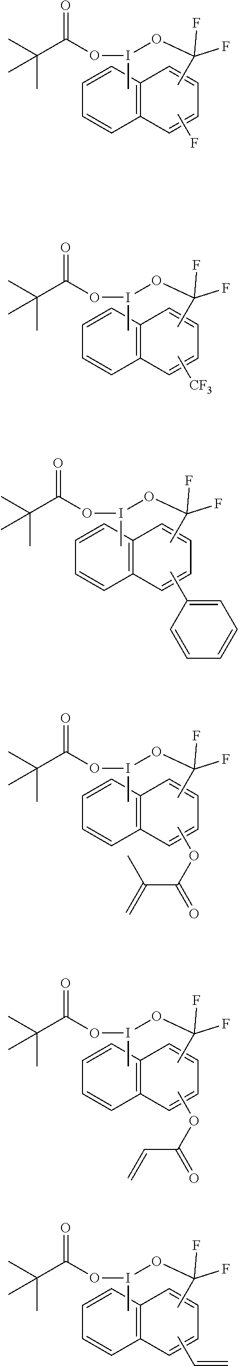

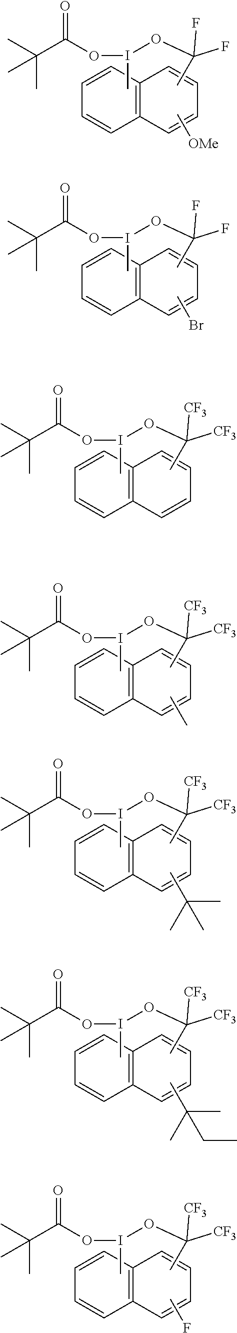

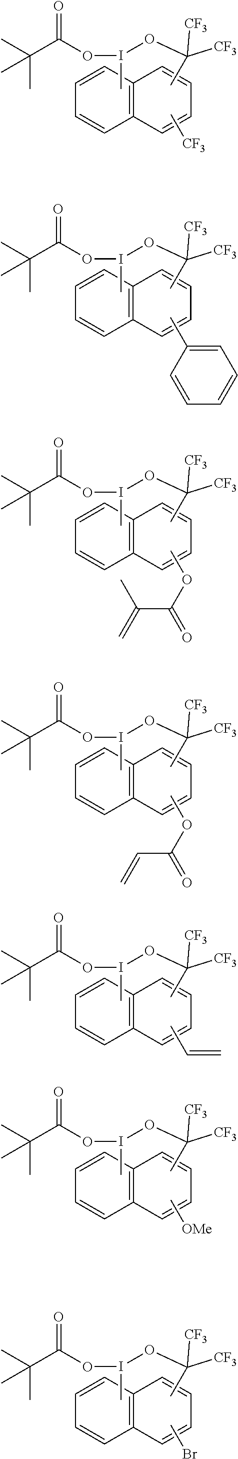

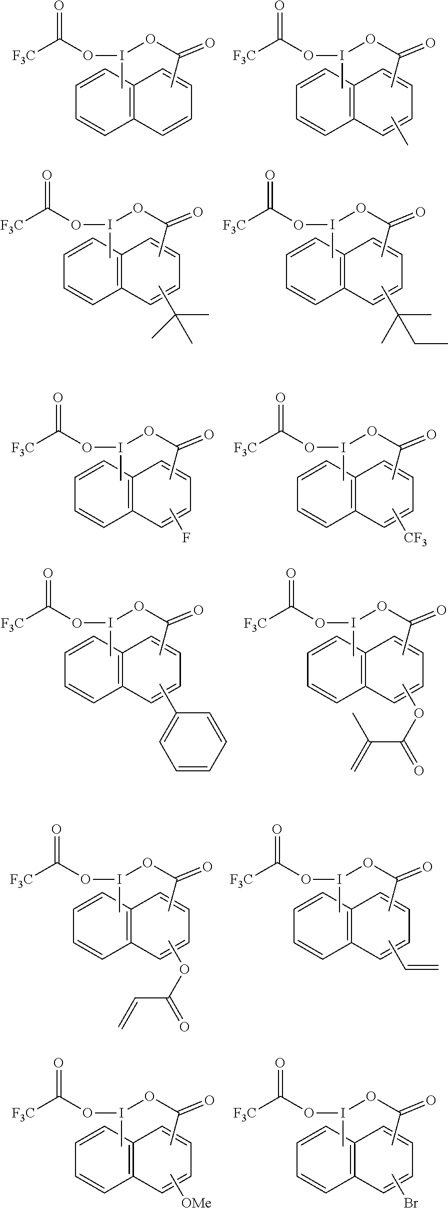

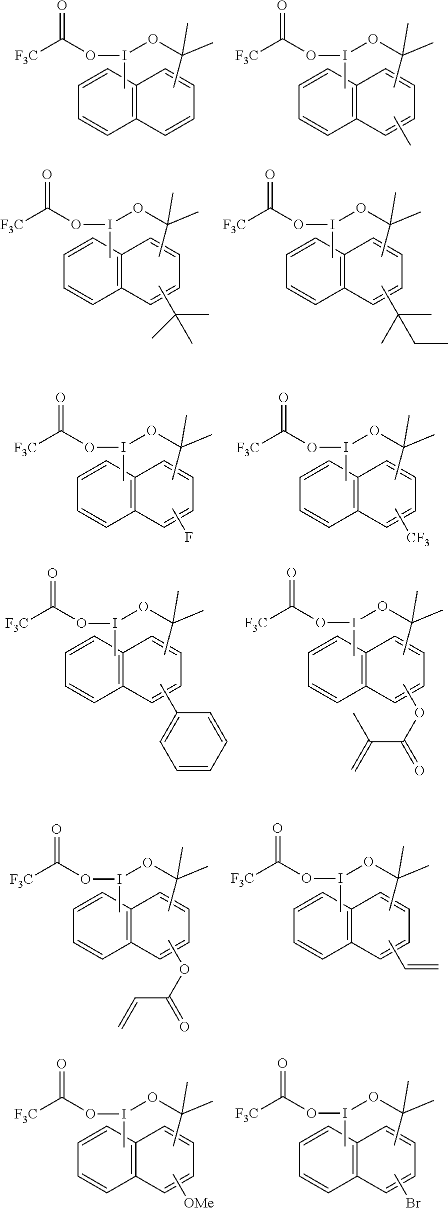

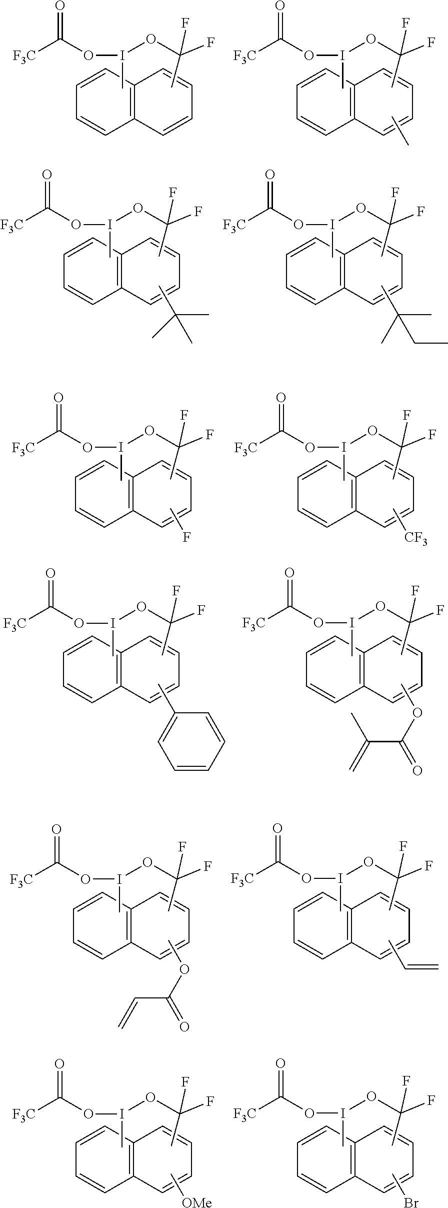

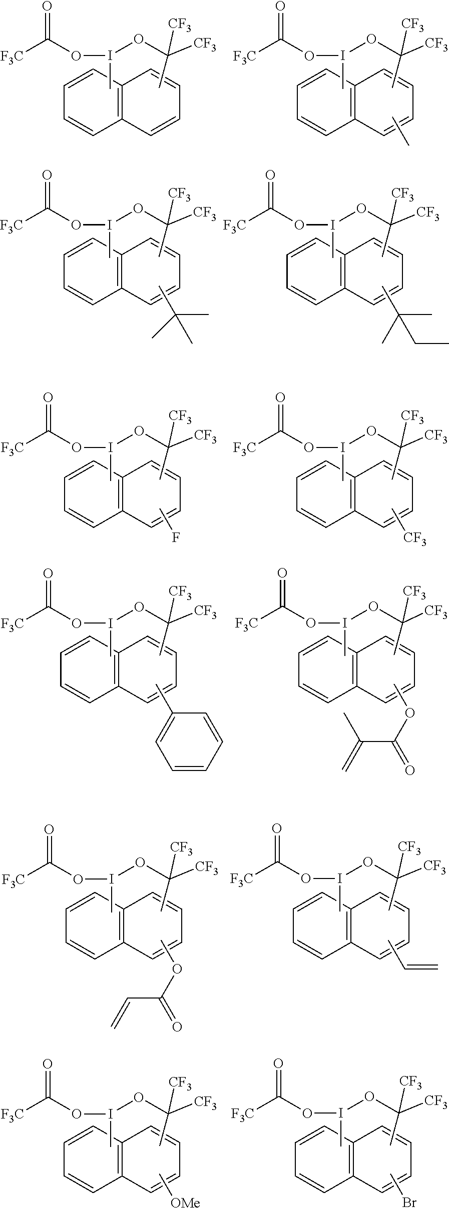

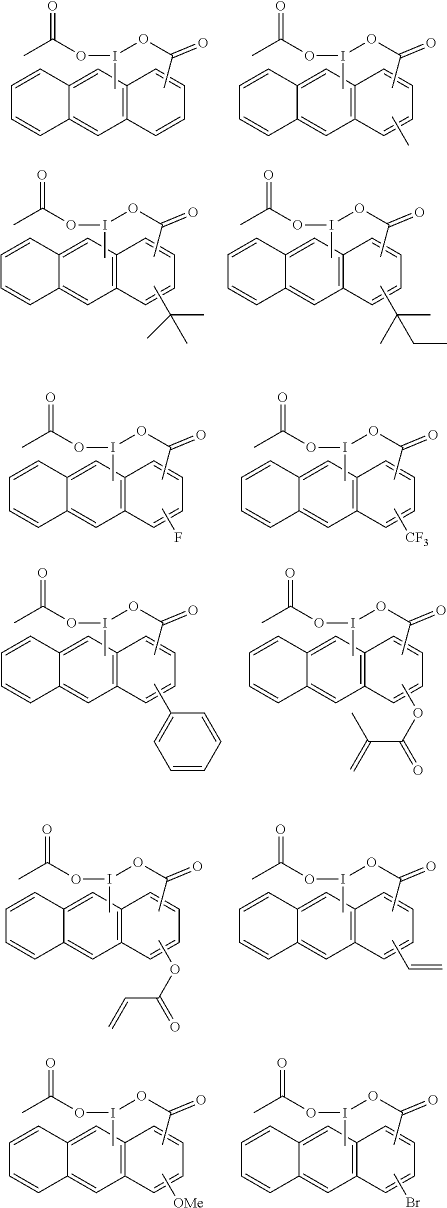

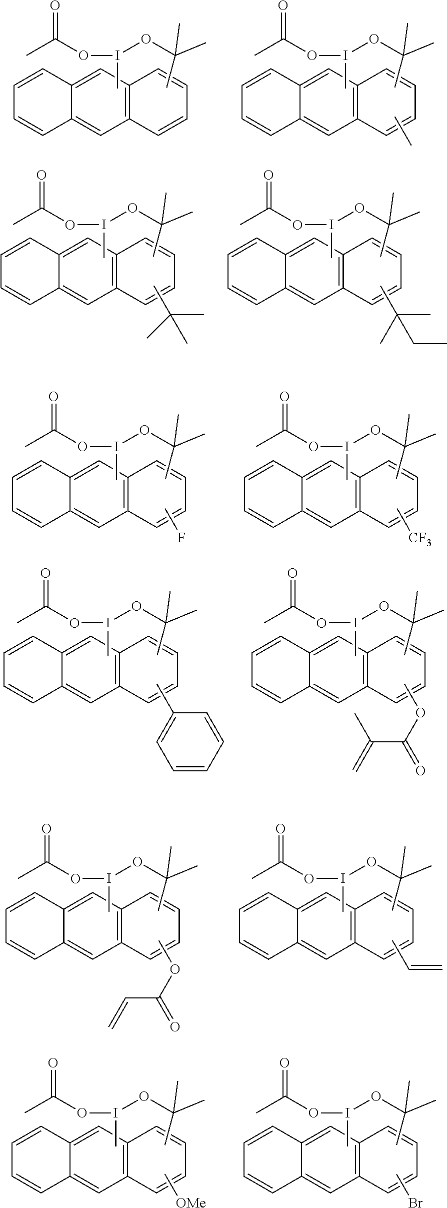

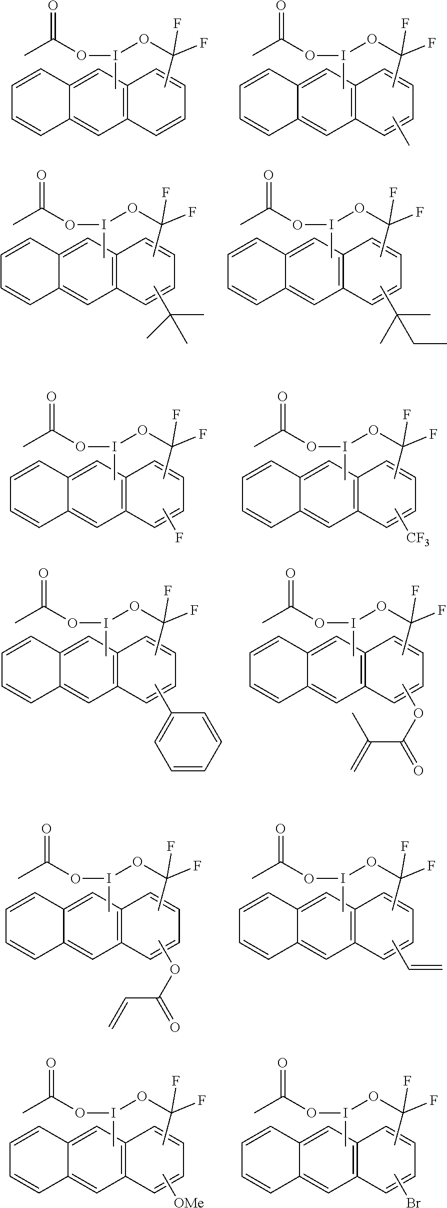

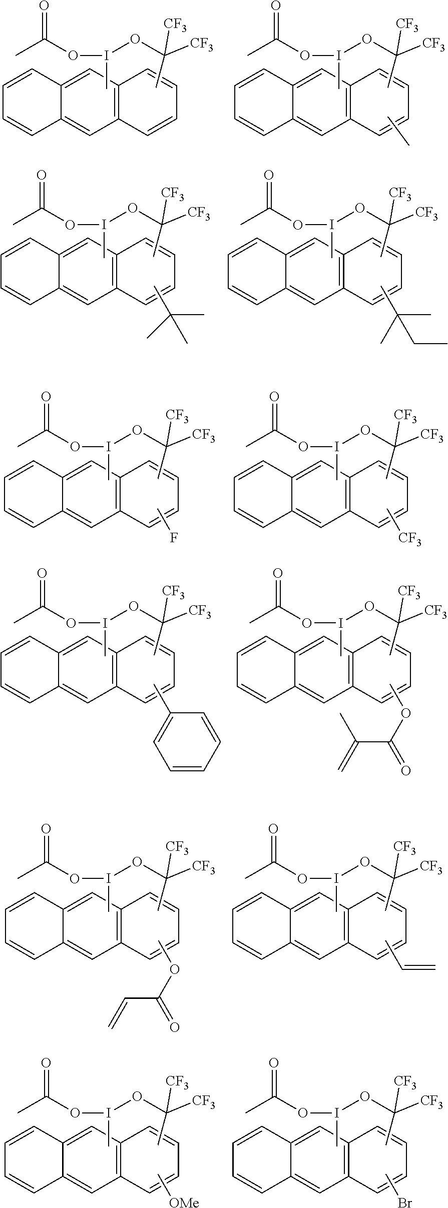

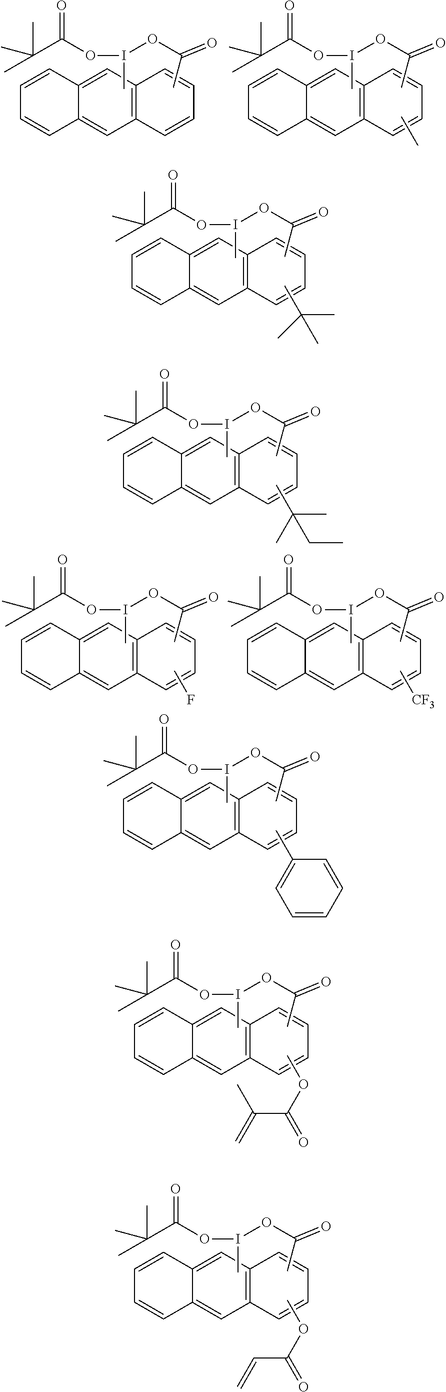

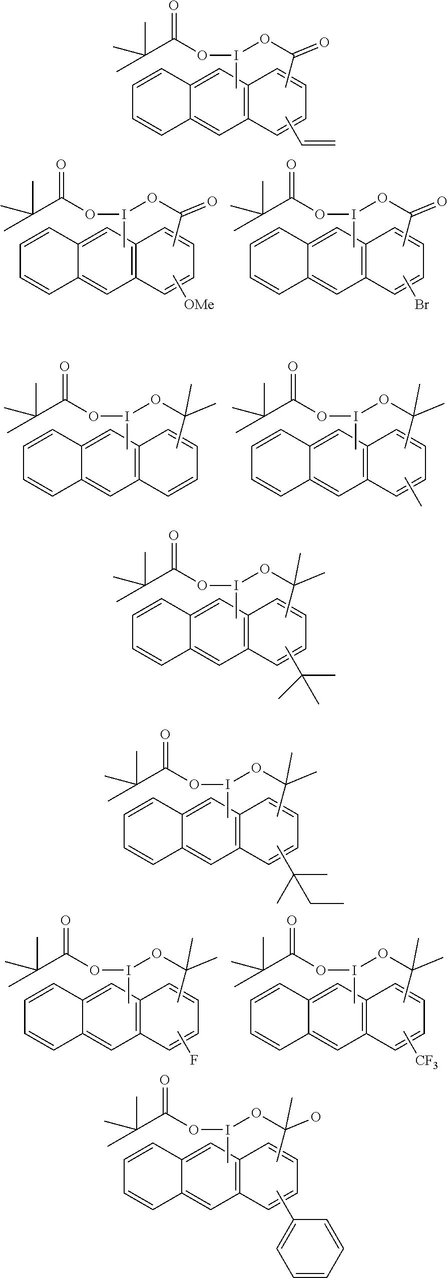

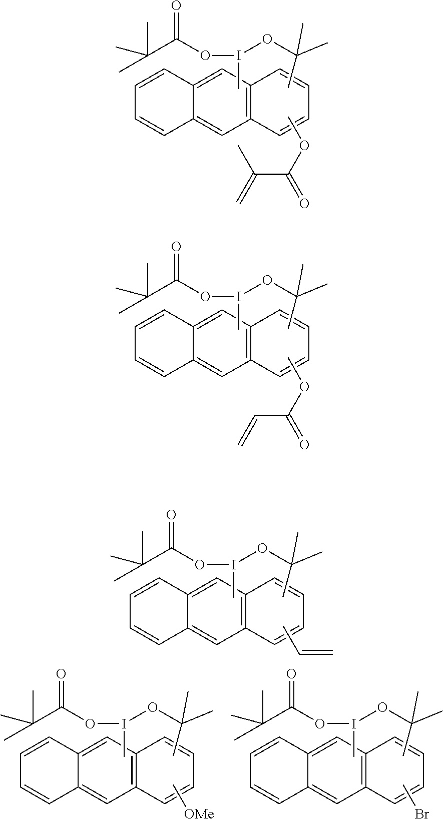

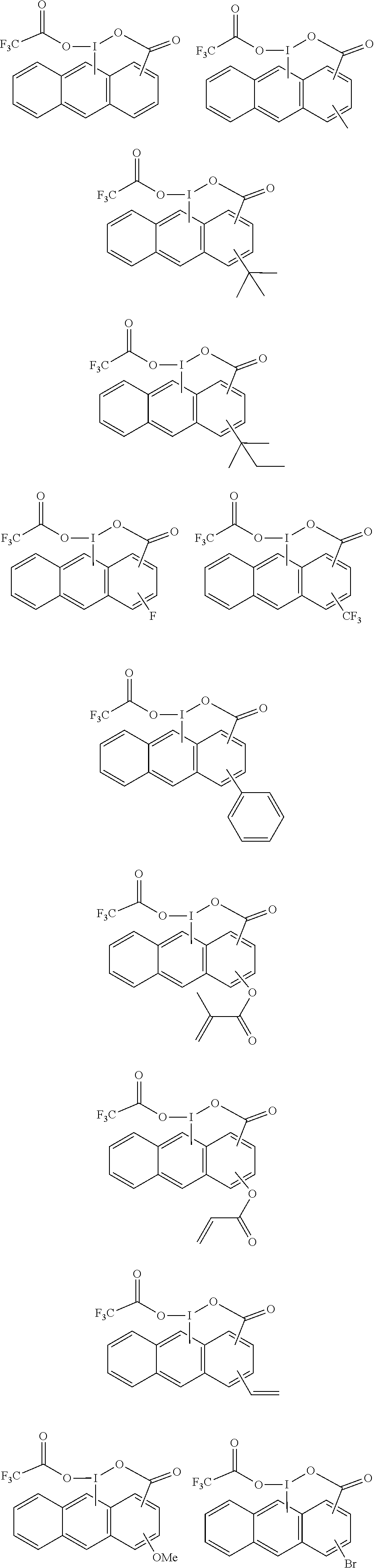

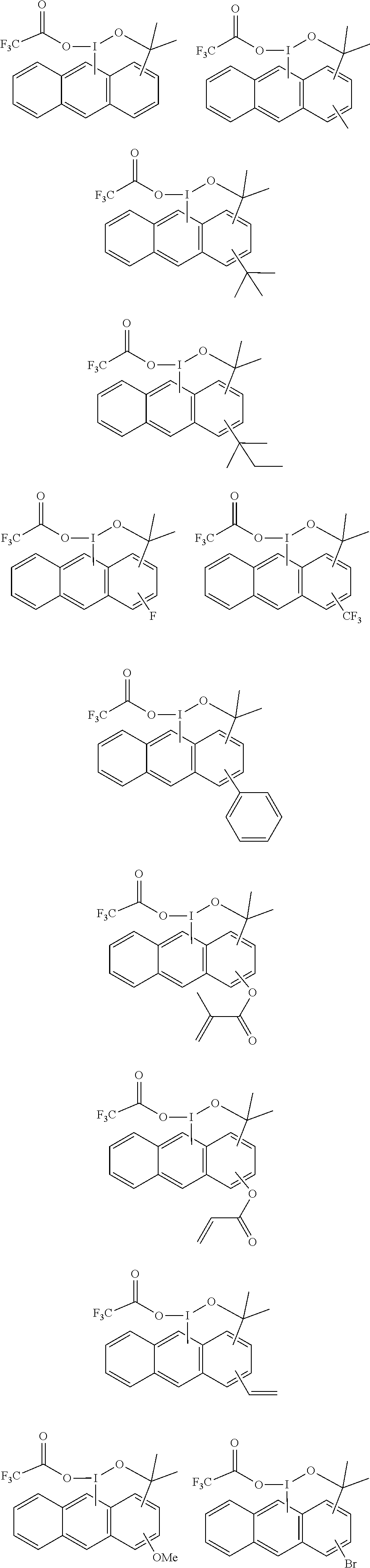

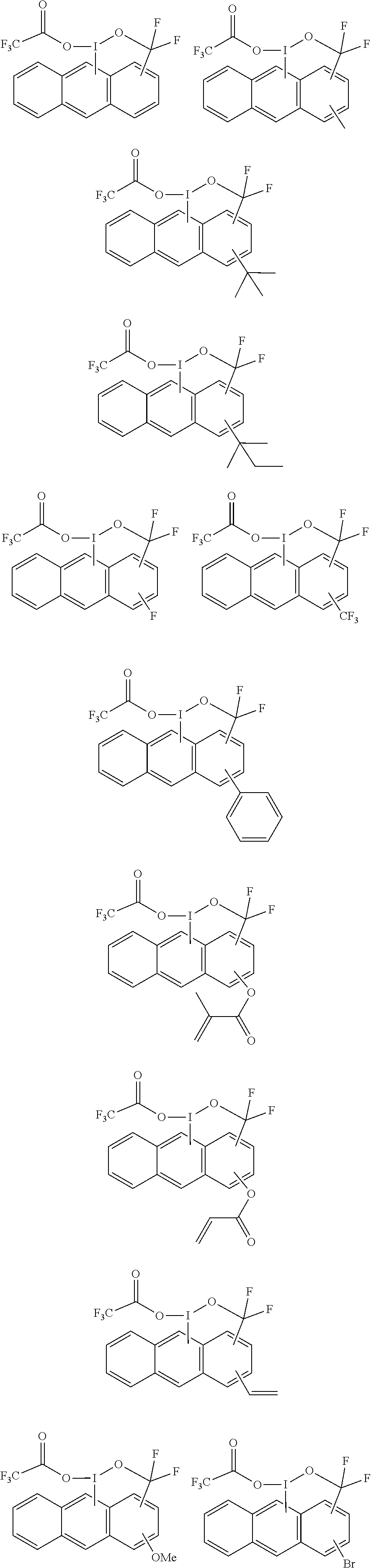

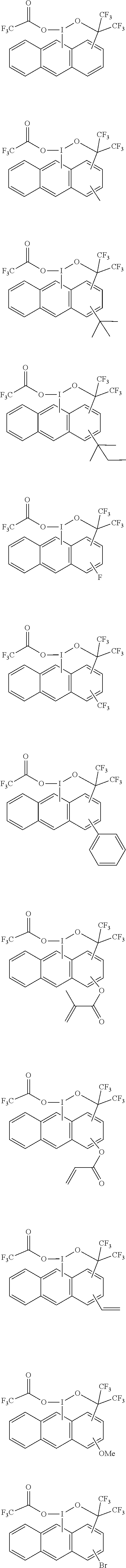

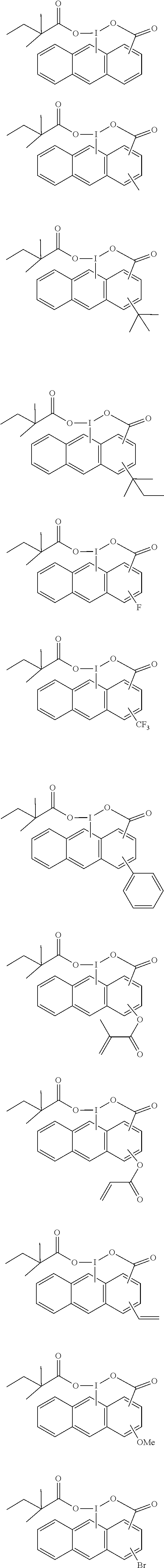

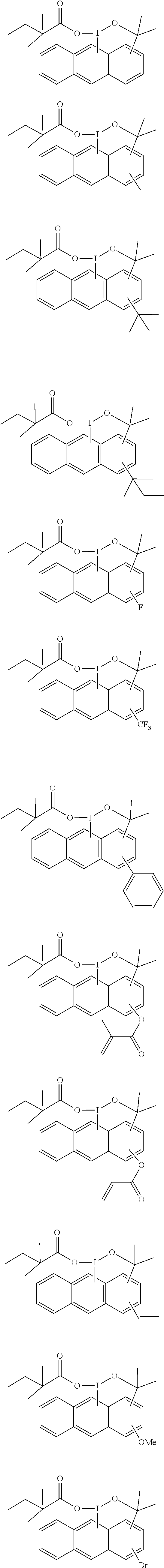

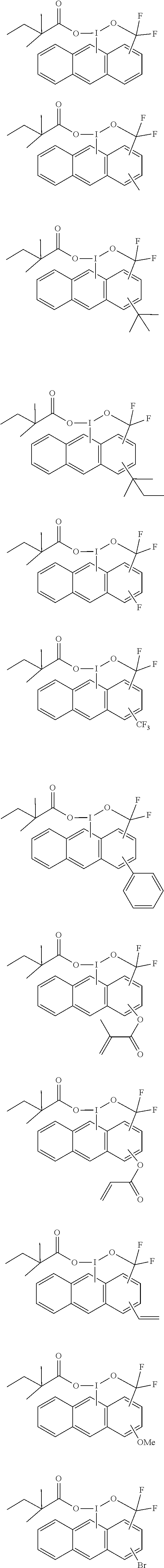

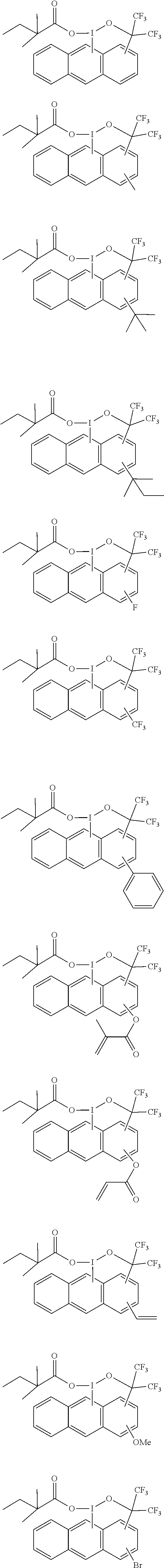

In the general formula (5), “m2” represents an integer of 0 to 2. When “m2” is 0, “n2” represents an integer of 1 to 3, “n3” represents an integer of 0 to 5, and 1≤(n2+n3)≤6 is satisfied. When “m2” is 1, “n2” represents an integer of 1 to 3, “n3” represents an integer of 0 to 7, and 1≤(n2+n3)≤8 is satisfied. When “m2” is 2, “n2” represents an integer of 1 to 3, “n3” represents an integer of 0 to 9, and 1≤(n2+n3)≤10 is satisfied.

In the general formula (5), R⁵¹ and R⁵² each independently represent a halogen atom or a hydrocarbyl group having 1 to 10 carbon atoms and optionally containing a heteroatom, the R⁵¹ and the R⁵² optionally being bonded to each other to form a ring together with the carbon atoms bonded thereto and the atoms between the carbon atoms. Examples of the halogen atom include a fluorine atom, a chlorine atom, a bromine atom, and an iodine atom. The hydrocarbyl group having 1 to 10 carbon atoms may be saturated or unsaturated, and may be linear, branched, or cyclic. Specific examples thereof include: alkyl groups having 1 to 10 carbon atoms, such as a methyl group, an ethyl group, a propyl group, an isopropyl group, an n-butyl group, an isobutyl group, a sec-butyl group, a tert-butyl group, an n-pentyl group, a tert-pentyl group, an n-hexyl group, an n-octyl group, a 2-ethylhexyl group, an n-nonyl group, and an n-decyl group; cyclic saturated hydrocarbyl groups having 3 to 10 carbon atoms, such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, a tricyclo[5.2.1.0^{2,6}]decanyl group, and an adamantyl group; alkenyl groups, such as a vinyl group and an allyl group; aryl groups having 6 to 10 carbon atoms, such as a phenyl group and a naphthyl group; and groups obtained by combining these groups. Furthermore, part or all of the hydrogen atoms of the hydrocarbyl group may be substituted with a group containing a heteroatom, such as an oxygen atom, a sulfur atom, a nitrogen atom, or a halogen atom, and part of the —CH₂— of the hydrocarbyl group may be substituted with a group containing a heteroatom such as an oxygen atom, a sulfur atom, or a nitrogen atom. The resulting hydrocarbyl group may contain a hydroxy group, a cyano group, a halogen atom, a carbonyl group, an ether bond, a thioether bond, an ester bond, a sulfonic acid ester bond, a carbonate bond, a carbamate bond, a lactone ring, a sultone ring, a carboxylic anhydride (—C(═O)—O—C(═O)—), etc. As the R⁵¹ and the R⁵², hydrocarbyl groups having 1 to 4 carbon atoms are preferable.

In the general formula (5), R⁵³ represents a halogen atom or a hydrocarbyl group having 1 to 40 carbon atoms and optionally containing a heteroatom. Examples of the halogen atom include a fluorine atom, a chlorine atom, a bromine atom, and an iodine atom. The hydrocarbyl group having 1 to 40 carbon atoms may be saturated or unsaturated, and may be linear, branched, or cyclic. Specific examples thereof include: alkyl groups having 1 to 40 carbon atoms, such as a methyl group, an ethyl group, a propyl group, an isopropyl group, an n-butyl group, an isobutyl group, a sec-butyl group, a tert-butyl group, an n-pentyl group, a tert-pentyl group, an n-hexyl group, an n-octyl group, a 2-ethylhexyl group, an n-nonyl group, and an n-decyl group; cyclic saturated hydrocarbyl groups having 3 to 40 carbon atoms, such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, a tricyclo[5.2.1.0^{2,6}]decanyl group, an adamantyl group, and an adamantylmethyl group; and aryl groups having 6 to 40 carbon atoms, such as a phenyl group, a naphthyl group, and an anthracenyl group. Furthermore, part or all of the hydrogen atoms of the hydrocarbyl group may be substituted with a group containing a heteroatom, such as an oxygen atom, a sulfur atom, a nitrogen atom, or a halogen atom, and part of the —CH₂— of the hydrocarbyl group may be substituted with a group containing a heteroatom such as an oxygen atom, a sulfur atom, or a nitrogen atom. The resulting hydrocarbyl group may contain a hydroxy group, a cyano group, a halogen atom, a carbonyl group, an ether bond, a thioether bond, an ester bond, a sulfonic acid ester bond, a carbonate bond, a carbamate bond, a lactone ring, a sultone ring, a carboxylic anhydride (—C(═O)—O—C(═O)—), etc. When “n3” is 2 to 9, the R⁵³'s are identical to or different from each other and the R⁵³'s are optionally bonded to each other to form a ring together with the carbon atoms of the aromatic ring bonded to the R⁵³'s.

Specific examples of the hypervalent iodine compound represented by the general formula (5) include the following, but are not limited thereto.

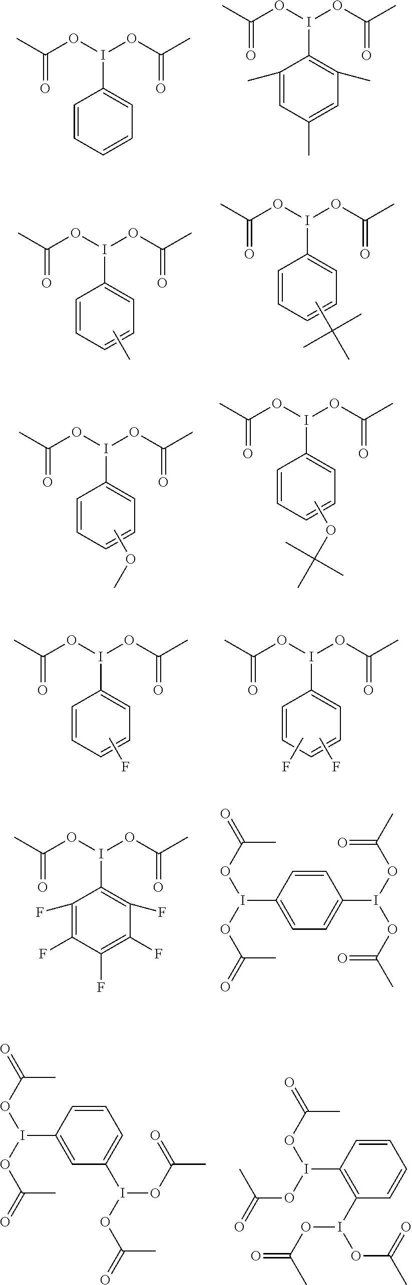

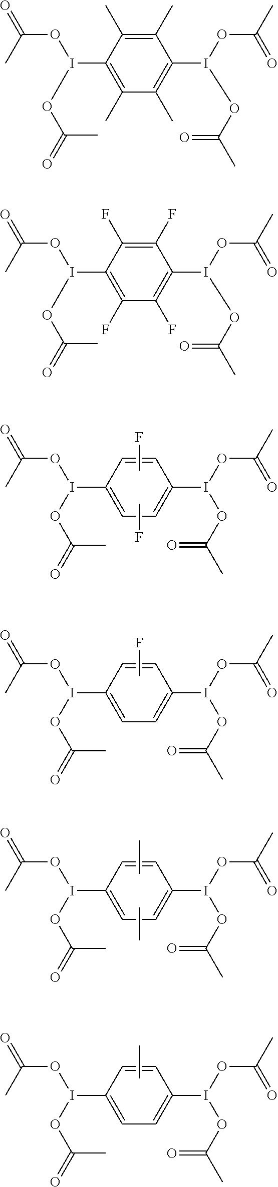

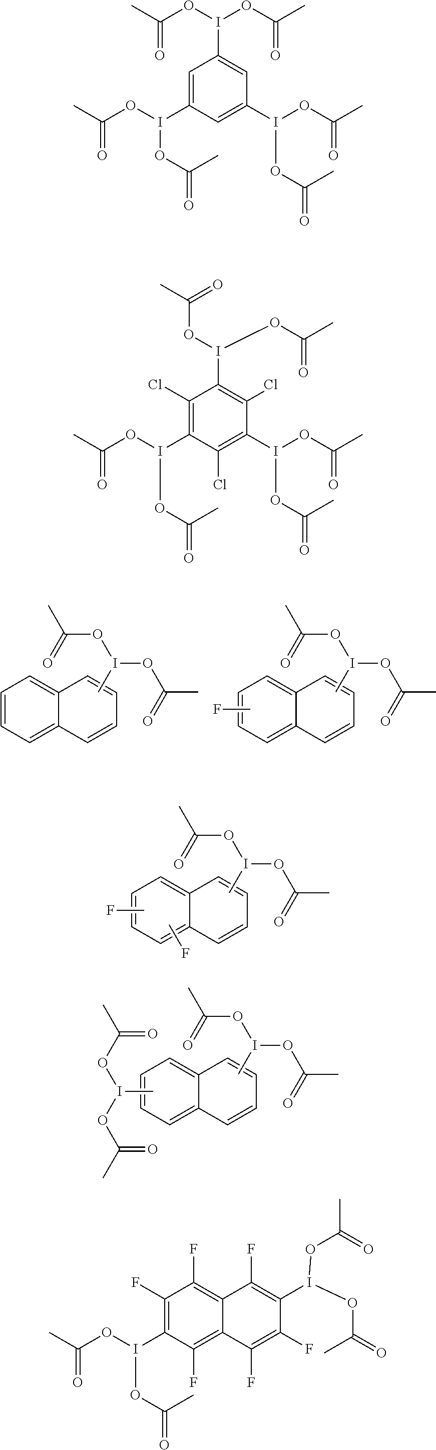

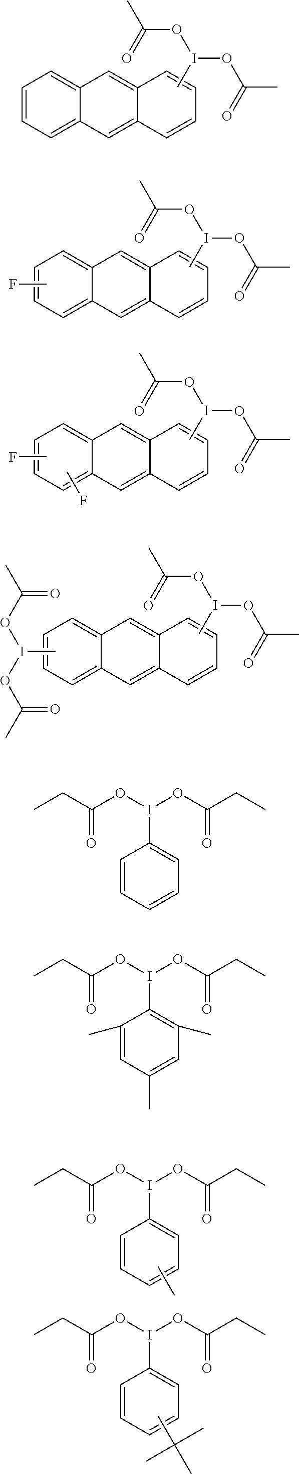

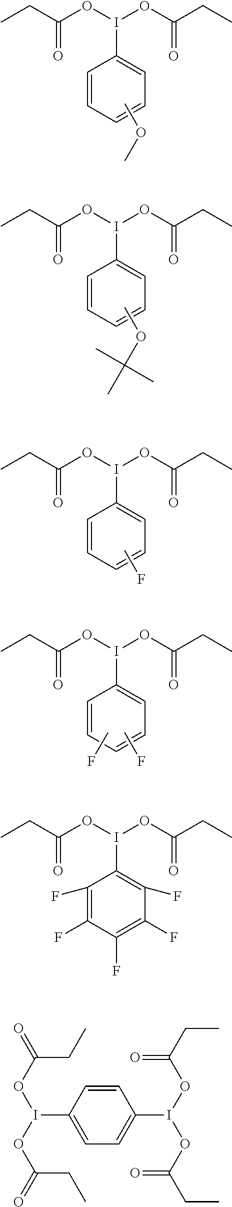

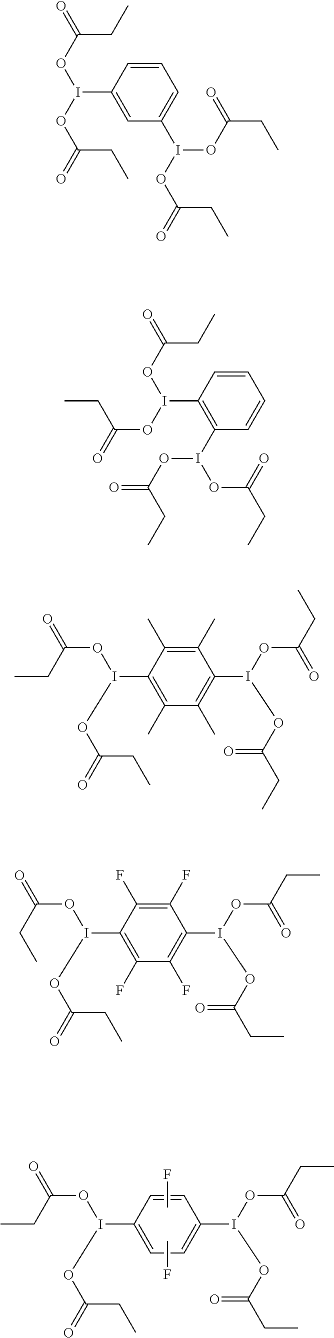

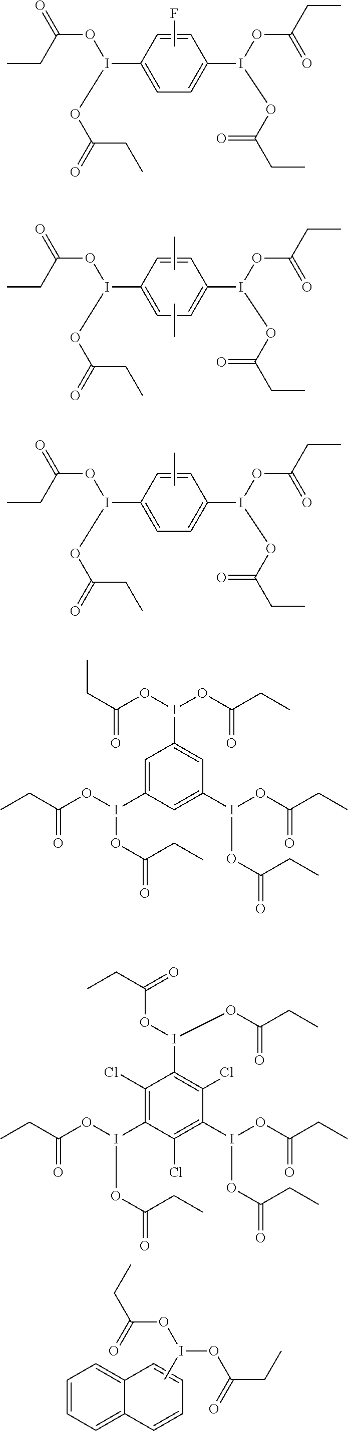

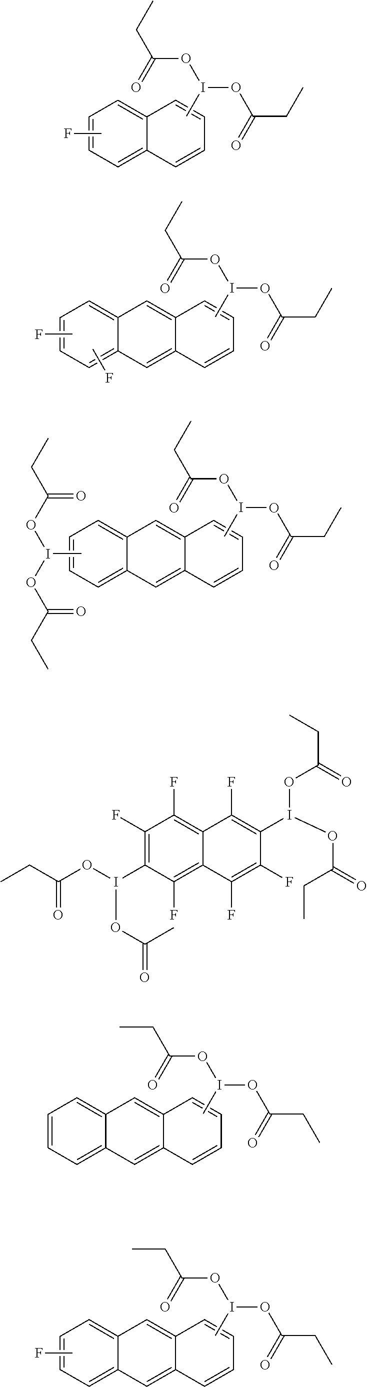

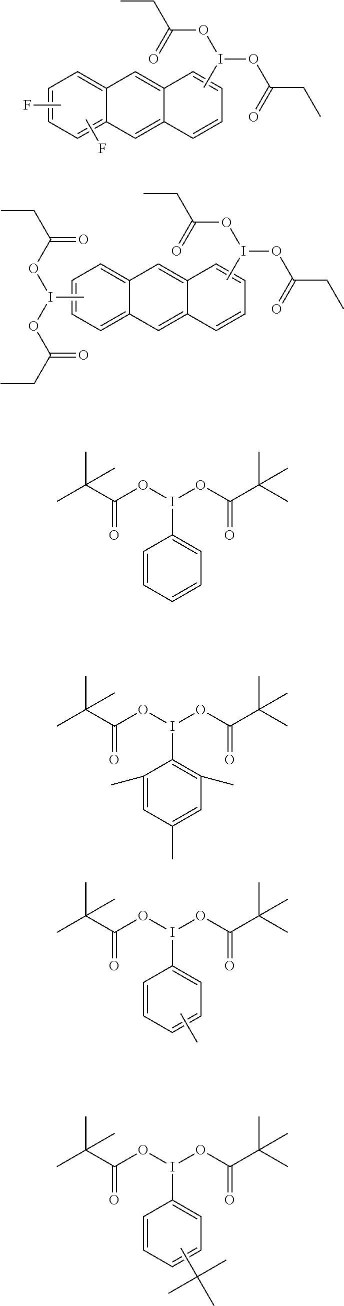

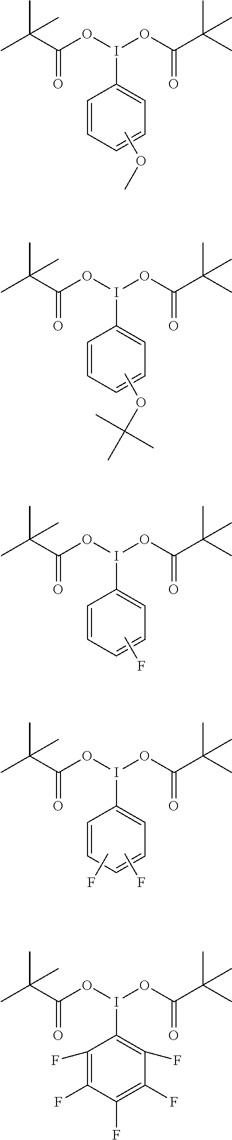

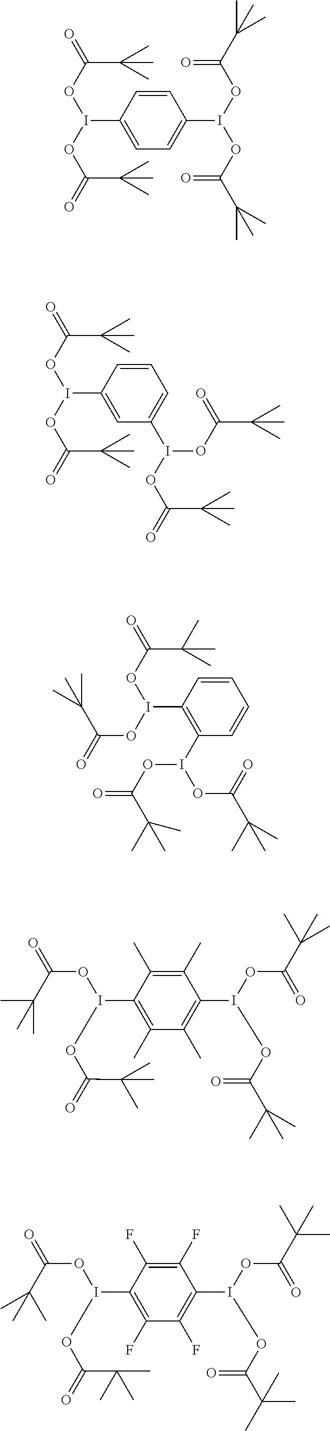

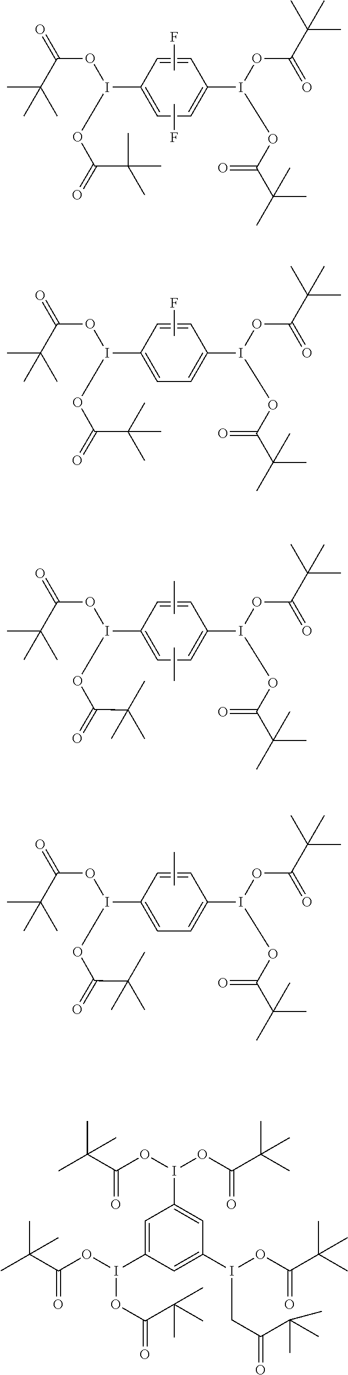

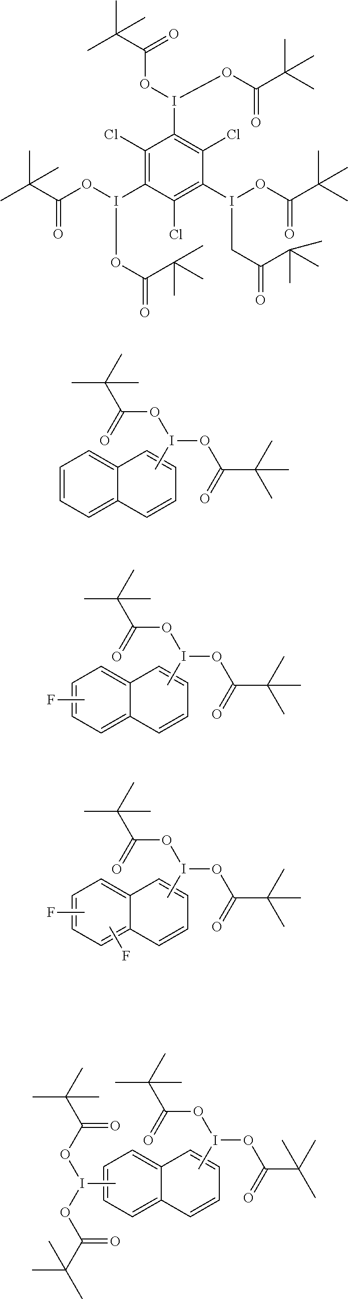

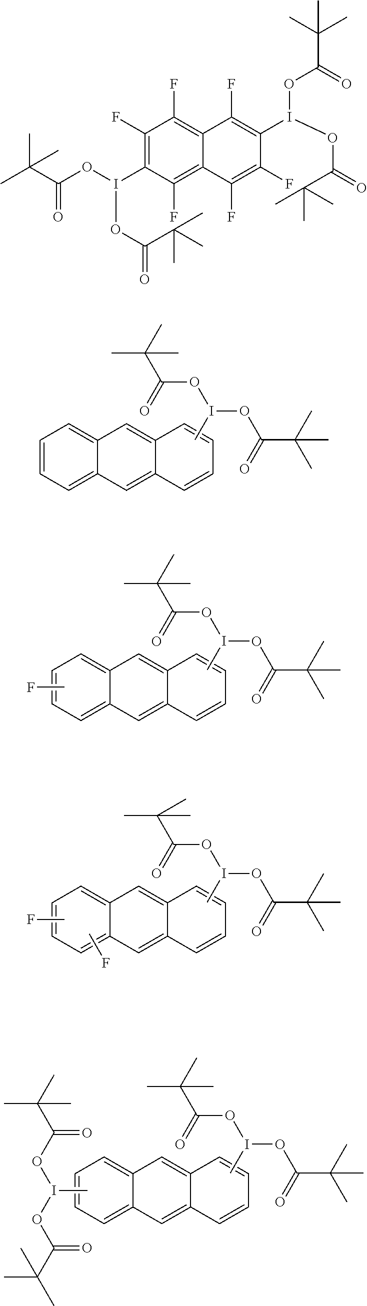

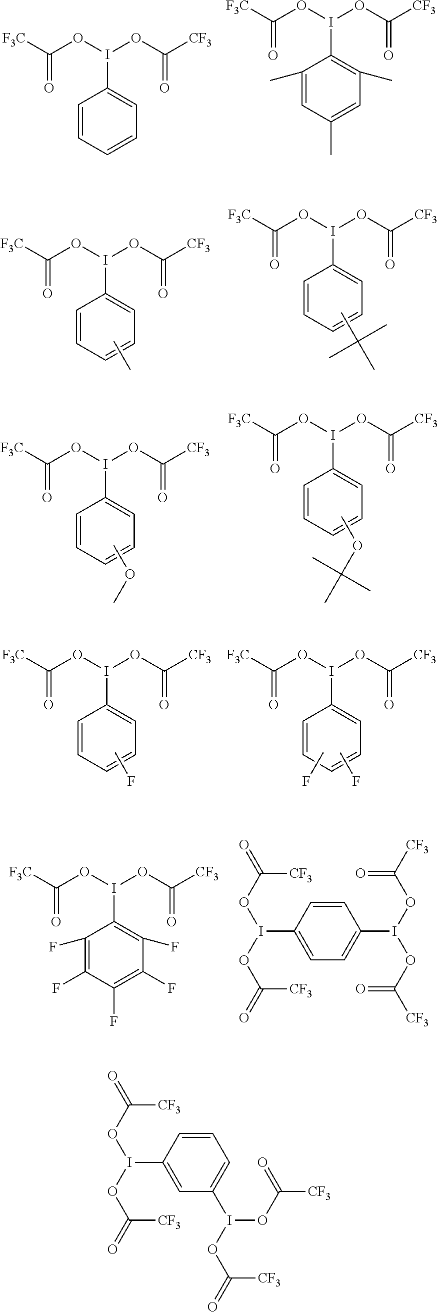

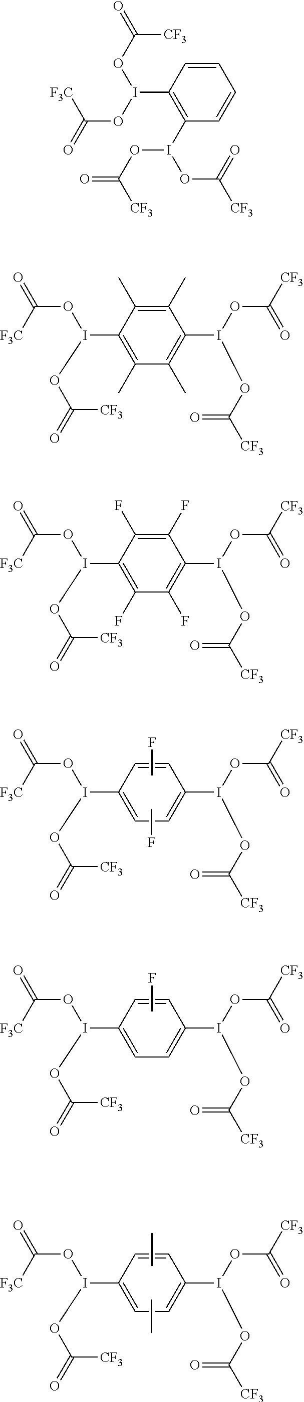

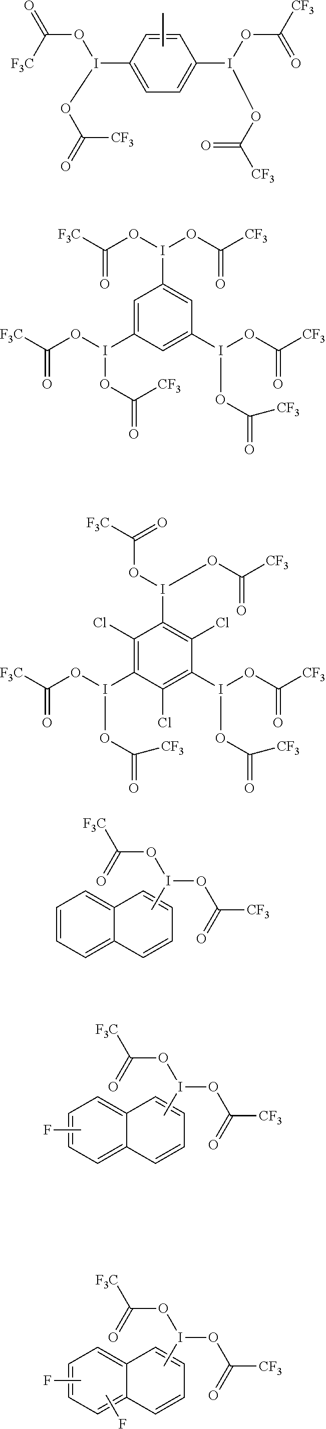

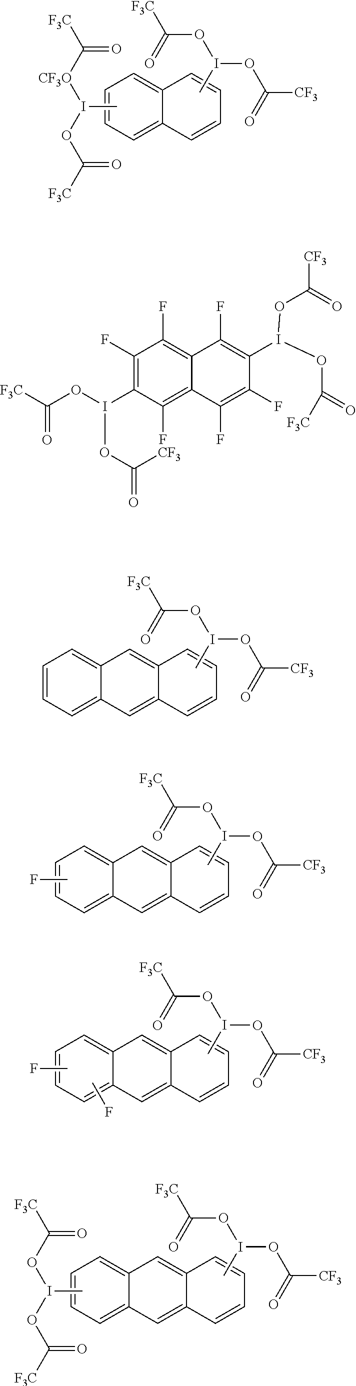

When the inventive resist composition contains other hypervalent iodine compounds, as the other hypervalent iodine compounds, the hypervalent iodine compound represented by the general formula (4) alone may be used, the hypervalent iodine compound represented by the general formula (5) alone may be used, or a combination of the hypervalent iodine compound represented by the general formula (4) and the hypervalent iodine compound represented by the general formula (5) may be used. Furthermore, regarding each of the hypervalent iodine compound represented by the general formula (4) and the hypervalent iodine compound represented by the general formula (5), one kind may be used, or two or more different kinds may be used in combination.

When the inventive resist composition contains other hypervalent iodine compounds as optional components, the other hypervalent iodine compounds are preferably contained in such an amount that “other hypervalent iodine compounds”:“hypervalent iodine compound represented by the general formula (1)=0:100 to 50:50, more preferably 0:100 to 10:90 in molar ratio.

### [Laminate]

The present invention is a laminate including:
a substrate; and
a resist film obtained from the above-described resist composition on the substrate.

In the inventive laminate, including a resist film obtained from the inventive resist composition, the resist film has high sensitivity, also exhibits excellent limiting resolution, is effective for precise fine processing, and in addition, is applicable to either positive or negative patterning. Therefore, the inventive laminate has a wide ranges of uses, and is highly useful in resist process technology.

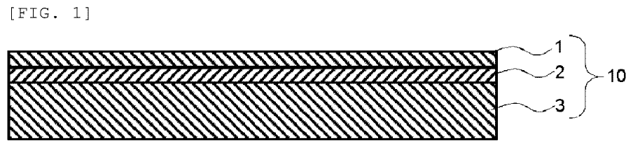

For example, like the laminate 10 shown in FIG. 1, the laminate may further include a resist underlayer film 2 between the substrate 3 and the resist film 1. That is, the above-described laminate can be one that further includes a resist underlayer film between the substrate and the resist film.

When a resist underlayer film is necessary for pattern formation, a resist underlayer film can also be disposed appropriately between the substrate and the resist film.

### [Patterning Process]

When the inventive resist composition is used for manufacturing various integrated circuits, a known lithography technique can be applied. Examples of patterning processes include a method including the steps of: forming a resist film by using the above-described resist composition on a substrate; exposing the resist film by using a high-energy beam; and developing the exposed resist film by using a developer as necessary.

That is, the present invention is a patterning process including the steps of:
forming a resist film by using the above-described resist composition on a substrate or on a resist underlayer film of a substrate on which the resist underlayer film has been laminated;
exposing the resist film by using a high-energy beam; and developing the exposed resist film by using a developer.

Firstly, the inventive resist composition is applied onto a substrate for manufacturing an integrated circuit, on a resist underlayer film of a substrate (Si, SiO₂, SiN, SiON, TiN, WSi, BPSG, SOG, organic antireflective film, etc.) on which the resist underlayer film has been laminated, on a substrate for manufacturing a mask circuit, or on a resist underlayer film of a substrate (Cr, Cro, CrON, MoSiz, SiOz, etc.) on which the resist underlayer film has been laminated, by an appropriate coating process, for example, spin coating, roll coating, flow coating, dip coating, spray coating, doctor coating, etc., so that the thickness of the coating film is 0.01 to 2 μm, for example. The coating film thus obtained is prebaked on a hot plate preferably at 60 to 200° C. for 10 seconds to 30 minutes, more preferably 80 to 180° C. for 30 seconds to 20 minutes, for example. Thus, a resist film can be formed. Note that a resist underlayer film means a film formed between the substrate and the resist film in a multilayer resist process. The resist underlayer film is not particularly limited, and a conventionally known film can be used.

Subsequently, the resist film is exposed by using a high-energy beam. Specific examples of the high-energy beam include ultraviolet ray (g-line (436 nm), h-line (405 nm), i-line (365 nm), etc.), deep ultraviolet ray, electron beam (EB), extreme ultraviolet ray (EUV), X-ray, soft X-ray, excimer laser beam (KrF excimer laser beam, ArF excimer laser beam, etc.), γ-ray, and synchrotron radiation. As the high-energy beam, it is preferable to use an i-line, a KrF excimer laser beam, an ArF excimer laser beam, an electron beam, or an extreme ultraviolet ray. When ultraviolet ray, deep ultraviolet ray, EUV, X-ray, soft X-ray, excimer laser beam, γ-ray, synchrotron radiation, or the like is employed as the high-energy beam, the irradiation is performed directly or while using a mask for forming a target pattern at an exposure dose of preferably about 1 to 300 mJ/cm², more preferably about 10 to 200 mJ/cm². When an EB is employed as the high-energy beam, the writing is performed directly or while using a mask for forming a target pattern at an exposure dose of preferably about 0.1 to 2000 μC/cm², more preferably about 0.5 to 1500 μC/cm². Note that the inventive resist composition is particularly suitable for fine patterning with an EB or EUV, among the high-energy beams.

After exposure, PEB is performed as necessary. In this case, PEB is preferably performed after the exposure on a hot plate or in an oven under the conditions of 30 to 200° C. for 10 seconds to 30 minutes, more preferably 60 to 180° C. for 30 seconds to 20 minutes.

After the exposure or after the PEB, development is performed by using a developer as necessary to perform patterning. Examples of the developer used in this event include: aqueous alkaline solutions, such as an aqueous solution of tetramethylammonium hydroxide; and organic solvents, such as 2-octanone, 2-nonanone, 2-heptanone, 3-heptanone, 4-heptanone, 2-hexanone, 3-hexanone, diisobutyl ketone, methylcyclohexanone, acetophenone, methylacetophenone, isopropyl alcohol, isoamyl alcohol, n-butanol, n-pentanol, cyclohexanol, propyl acetate, butyl acetate, isobutyl acetate, pentyl acetate, butenyl acetate, isopentyl acetate, cyclohexyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, phenylmethyl acetate, phenylethyl acetate, benzyl formate, phenylethyl formate, methyl 3-phenylpropionate, benzyl propionate, 2-phenylethyl acetate, 1-propanol, 3-methoxybutanol, 3-methyl-3-methoxybutanol, 1-methoxy-2-propanol, 1-ethoxy-2-propanol, diacetone alcohol, and 4-methyl-2-pentanol. One kind of these developers may be used, or two or more kinds thereof may be used in mixture.

After the development, rinsing is performed as necessary. The rinsing liquid is preferably a solvent that is miscible with the developer but does not dissolve the resist film. As such a solvent, it is preferable to use an alcohol having 3 to 10 carbon atoms, an ether compound having 8 to 12 carbon atoms, and an alkane, alkene, alkyne, and aromatic solvent, each having 6 to 12 carbon atoms. Alternatively, water may be used instead of an organic solvent as a rinsing liquid.

The rinsing can reduce resist pattern collapse and defect formation. Meanwhile, the rinsing is not necessarily essential, and the amount of the solvent used can be reduced by not performing the rinsing.

In the inventive resist composition, a difference occurs in the solubility between exposed portions and unexposed portions by virtue of exposure, and a positive or negative pattern can be formed. Therefore, it is possible to use a developer that dissolves exposed portions and does not dissolve unexposed portions, or a developer that dissolves unexposed portions and does not dissolve exposed portions. Thus, the inventive patterning process makes it possible to form a positive or negative pattern by appropriately selecting a developer, and therefore, is widely applicable to various kinds of fine patterning.

### EXAMPLES

Hereinafter, the present invention will be specifically described with reference to Synthesis Examples, Examples, and Comparative Examples, but the present invention is not limited to the Examples.

### [1] Synthesis of Hypervalent Iodine Compound

The hypervalent iodine compounds used in the Examples are represented by the following formulae (I-1) and (I-2). The Ph in the formula represents a phenyl group.

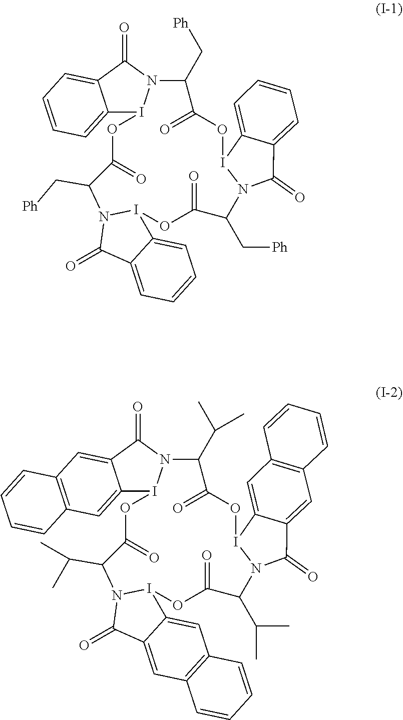 [SMILES: O=C1OI2c3ccccc3C(=O)N2C(Cc2ccccc2)C(=O)OI2c3ccccc3C(=O)N2C(Cc2ccccc2)C(=O)OI2c3ccccc3C(=O)N2C1Cc1ccccc1 ; CC(C)C1C(=O)OI2c3cc4ccccc4cc3C(=O)N2C(C(C)C)C(=O)OI2c3cc4ccccc4cc3C(=O)N2C(C(C)C)C(=O)OI2c3cc4ccccc4cc3C(=O)N12]

The hypervalent iodine compound represented by the formula (I-1) was synthesized with reference to ChemRxiv, 2024, 10.26434/chemrxiv-2024-11j6h. The hypervalent iodine compound represented by the formula (1-2) was synthesized with reference to J. Mater. Chem. C, 2025, 13, 842.

### [2] Synthesis of Polymers

The monomers used in the Comparative Examples are represented by the following formulae (c-1) and (c-2).

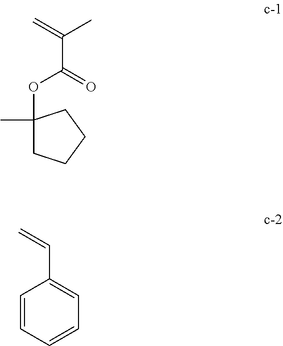

Under a nitrogen atmosphere, a monomer c-1 (101 g), a monomer c-2 (42 g), 5.4 g of V-601 (manufactured by FUJIFILM Wako Pure Chemical Corporation), and 180 g of MEK were added into a flask to prepare a monomer-polymerization initiator solution. Into another flask with a nitrogen atmosphere, 55 g of MEK was added and heated to 80° C. with stirring, and then the monomer-polymerization initiator solution was added dropwise over 4 hours. After the dropwise addition, the polymerization liquid was further stirred for 2 hours with maintaining the temperature at 80° C., and then cooled to room temperature. The obtained polymerization liquid was added dropwise to 4000 g of vigorously stirred hexane, and a precipitated polymer was collected by filtration. Furthermore, the obtained polymer was washed twice with 1200 g of hexane, and then dried in vacuo at 50° C. for 20 hours to obtain a white powder polymer P-10 (137 g, 96% yield). In the polymer P-10, the ratio of the repeating unit derived from c-1 to the repeating unit derived from c-2 was 60:40 in molar ratio. The polymer P-10 had Mw of 9800 and Mw/Mn of 1.82. The Mw is a polystyrene-converted measurement value obtained by GPC using THE as an eluent.

### [3] Preparation of Resist Compositions

### Examples 1-1 to 1-4 and Comparative Examples 1-1 to 1-3

Hypervalent iodine compounds and additives were dissolved in a solvent containing 0.01 mass % of a surfactant (PF-636, manufactured by Omnova Solutions Inc.) in the constitution shown below in Table 1, and the obtained solution was filtered with a 0.2-μm Teflon (registered trademark) filter to prepare each of resist compositions (R-01 to R-04) and a comparative resist composition (CR-01). Meanwhile, a polymer, a photo-acid generator, and a sensitivity modifier were dissolved in a solvent containing 0.01 mass % of a surfactant (PF-636, manufactured by Omnova Solutions Inc.) in the constitution shown below in Table 2, and the obtained solution was filtered with a 0.2-μm Teflon (registered trademark) filter to prepare each of comparative resist compositions (CR-02 and CR-03).

TABLE 1

| | | Hypervalent | | | |
|---|---|---|---|---|---|
| | | iodine | | | |
| | | compound | Additive | Solvent 1 | Solvent 2 |
| | Resist | (parts by | (parts by | (parts by | (parts by |
| | composition | mass) | mass) | mass) | mass) |
| Example 1-1 | R-01 | I-1(10) | — | PGMEA(400) | AcOH(100) |
| Example 1-2 | R-02 | I-2(10) | — | HBM(400) | PA(100) |
| Example 1-3 | R-03 | I-1(10) | o-1(1) | PGMEA(400) | AcOH(100) |
| Example 1-4 | R-04 | I-1(10) | m-1(1) | PGMEA(400) | AcOH(100) |
| Comparative | CR-01 | — | o-1(11.5) | PGMEA(900) | AcOH(100) |
| Example 1-1 | | | m-1(8.6) | | |

TABLE 2

| | | | Photo-acid | Sensitivity | | |
|---|---|---|---|---|---|---|
| | | Polymer | generator | modifier | Solvent 1 | Solvent 2 |
| | Resist | (parts by | (parts by | (parts by | (parts by | (parts by |
| | composition | mass) | mass) | mass) | mass) | mass) |
| Comparative | CR-02 | P-10(80) | PAG-1(19) | Q-1(6) | PGMEA(1890) | GBL(210) |
| Example 1-2 | | | | | | |
| Comparative | CR-03 | P-10(80) | PAG-1(19) | Q-1(5) | PGMEA(1890) | GBL(210) |
| Example 1-3 | | | | | | |

In Tables 1 and 2, additives O-1 and m-1, photo-acid generator PAG-1, sensitivity modifier Q-1, and solvents are as follows.

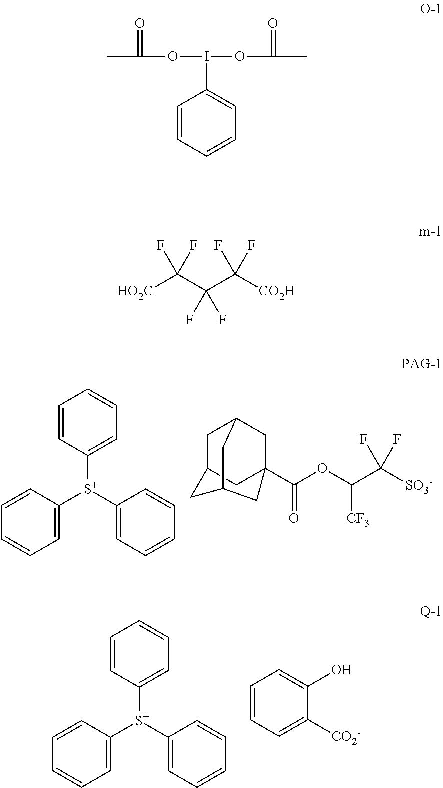

### Solvents:

PGMEA (propylene glycol monomethyl ether acetate)
AcOH (acetic acid)
HBM (methyl 2-hydroxyisobutyrate)
PA (propionic acid)
GBL (γ-butyrolactone)

### [4] EUV Lithography Evaluation (Line-and-Space Pattern, Positive Tone Development)

### Examples 2-1 to 2-4 and Comparative Examples 2-1 to 2-3

Each of the resist compositions (R-01 to R-04 and CR-01 to CR-03) was applied by spin-coating on a Si substrate on which a silicon-containing spin-on hard mask SHB-A940, manufactured by Shin-Etsu Chemical Co., Ltd. (silicon content of 43 mass %), was formed with 20 nm in film thickness, and subjected to post apply bake (PAB) by using a hot plate at the temperature shown in Table 3 for 60 seconds to form a resist film having a film thickness of 40 nm. Each of the formed resist films was exposed using an EUV scanner NXE3400 (NA 0.33, σ 0.9, 90° dipole illumination), manufactured by ASML Holding N.V., to form a 36-nm 1:1 line-and-space (LS) pattern. Then, PEB was performed on a hot plate at the temperature shown in Table 3 for 60 seconds, and then development was performed with the developer shown in Table 3 for 30 seconds to form an LS pattern having a space width of 18 nm and a pitch of 36 nm.

Regarding the obtained resist pattern, the following evaluations were carried out. The results are shown in Table 3.

### [Sensitivity Evaluation]

The LS pattern was observed using a length-measurement SEM (CG-6300), manufactured by Hitachi High-Technologies Corporation, and an optimum exposure dose Eop (mJ/cm²) to yield the LS pattern with 18 nm in space width and 36 nm in pitch was determined to specify this value as sensitivity.

### [LWR Evaluation]

In the LS pattern obtained by irradiation at the optimum exposure dose, sizes in ten positions in the longitudinal direction of the space width were measured with a length-measurement SEM (CG-6300), manufactured by Hitachi High-Technologies Corporation. From the results, a tripled value (3σ) of a standard variation (σ) was determined as LWR. A smaller LWR value can yield a pattern with lower roughness and a more uniform space width.

### [Limiting Resolution Evaluation]

A pattern was formed while gradually increasing the exposure dose from the optimum exposure dose at which the LS pattern can be formed, and in this event, the limit of the line width (nm) at which resolution was possible was determined using a length-measurement SEM (CG-6300), manufactured by Hitachi High-Technologies Corporation, to specify this value as limiting resolution (nm). A smaller value indicates that it is possible to form a finer pattern with better limiting resolution.

TABLE 3

| | | | | | | Limiting |
|---|---|---|---|---|---|---|
| | Resist | PAB/PEB | | Eop | LWR | resolution |
| | composition | (° C.) | Developer | (mJ/cm²) | (nm) | (nm) |
| Example 2-1 | R-01 | 130/90 | nBA | 24 | 1.3 | 7 |
| Example 2-2 | R-02 | 130/90 | nBA | 25 | 1.5 | 8 |
| Example 2-3 | R-03 | 130/90 | nBA | 20 | 1.7 | 9 |
| Example 2-4 | R-04 | 130/90 | nBA | 35 | 1.6 | 9 |
| Comparative | CR-01 | 130/90 | nBA | 56 | 2.9 | 16 |
| Example 2-1 | | | | | | |
| Comparative | CR-02 | 105/90 | TMAH | 80 | 4.5 | 18 |
| Example 2-2 | | | | | | |
| Comparative | CR-03 | 105/90 | TMAH | 85 | 4.8 | 18 |
| Example 2-3 | | | | | | |

### Developers:

nBA (butyl acetate)
TMAH (2.38 mass % aqueous solution of tetramethylammonium hydroxide)

### [5] EUV Lithography Evaluation (Line-and-Space Pattern, Negative Tone Development)

### Examples 3-1 to 3-4 and Comparative Examples 3-1 to 3-3

Each of the resist compositions (R-01 to R-04 and CR-01 to CR-03) was applied by spin-coating on a Si substrate on which a silicon-containing spin-on hard mask SHB-A940, manufactured by Shin-Etsu Chemical Co., Ltd. (silicon content of 43 mass %), was formed with 20 nm in film thickness, and subjected to post apply bake (PAB) by using a hot plate at the temperature shown in Table 4 for 60 seconds to form a resist film having a film thickness of 40 nm. Each of the formed resist films was exposed using an EUV scanner NXE3400 (NA 0.33, 0.9, 90° dipole illumination), manufactured by ASML Holding N.V., to form a 36-nm 1:1 line-and-space (LS) pattern. Then, baking (PEB) was performed on a hot plate at the temperature shown in Table 4 for 60 seconds, and then development was performed with the developer shown in Table 4 for 30 seconds to form an LS pattern having a space width of 18 nm and a pitch of 36 nm.

Regarding the obtained resist pattern, the following evaluations were carried out. The results are shown in Table 4.

### [Sensitivity Evaluation]

The LS pattern was observed using a length-measurement SEM (CG-6300), manufactured by Hitachi High-Technologies Corporation, and an optimum exposure dose Eop (mJ/cm²) to yield the LS pattern with 18 nm in space width and 36 nm in pitch was determined to specify this value as sensitivity.

### [LWR Evaluation]

In the LS pattern obtained by irradiation at the optimum exposure dose, sizes in ten positions in the longitudinal direction of the space width were measured with a length-measurement SEM (CG-6300), manufactured by Hitachi High-Technologies Corporation. From the results, a tripled value (3σ) of a standard variation (σ) was determined as LWR. A smaller LWR value can yield a pattern with lower roughness and a more uniform space width.

### [Limiting Resolution Evaluation]

A pattern was formed while gradually increasing the exposure dose from the optimum exposure dose at which the LS pattern can be formed, and in this event, the limit of the line width (nm) at which resolution was possible was determined using a length-measurement SEM (CG-6300), manufactured by Hitachi High-Technologies Corporation, to specify this value as limiting resolution (nm). A smaller value indicates that it is possible to form a finer pattern with better limiting resolution.

TABLE 4

| | | | | | | Limiting |
|---|---|---|---|---|---|---|
| | Resist | PAB/PEB | | Eop | LWR | resolution |
| | composition | (° C.) | Developer | (mJ/cm²) | (nm) | ( nm ) |
| Example 3-1 | R-01 | 130/90 | TMAH | 25 | 1.4 | 7 |
| Example 3-2 | R-02 | 130/90 | TMAH | 25 | 1.5 | 8 |
| Example 3-3 | R-03 | 130/90 | TMAH | 22 | 1.8 | 10 |
| Example 3-4 | R-04 | 130/90 | TMAH | 36 | 1.9 | 11 |
| Comparative | CR-01 | 130/90 | TMAH | 57 | 3.2 | 18 |
| Example 3-1 | | | | | | |
| Comparative | CR-02 | 105/90 | nBA | 83 | 4.6 | 18 |
| Example 3-2 | | | | | | |
| Comparative | CR-03 | 105/90 | nBA | 86 | 4.9 | 18 |
| Example 3-3 | | | | | | |

From the results shown in Tables 3 and 4, it was found that the inventive resist composition was excellent in sensitivity, LWR, and resolution in the formation of line-and-space patterns by EUV exposure in cases of either positive tone or negative tone development.

### [6] EUV Lithography Evaluation (Contact Hole Pattern)

### Examples 4-1 to 4-4 and Comparative Examples 4-1 to 4-3

Each of the resist compositions (R-01 to R-04 and CR-01 to CR-03) was respectively applied by spin-coating on a Si substrate on which a silicon-containing spin-on hard mask SHB-A940, manufactured by Shin-Etsu Chemical Co., Ltd. (silicon content of 43 mass %), was formed with 20 nm in film thickness. Then, post apply bake (PAB) was performed at the temperature shown in Table 5 for 60 seconds using a hot plate to produce a resist film with 50 nm in film thickness. Subsequently, the resist film was exposed using an EUV scanner NXE3400 (NA 0.33, σ 0.9/0.6, quadrupole illumination, 64 nm in pitch on wafer size, hole pattern mask with +20% bias), manufactured by ASML Holding N.V. Then, baking (PEB) was performed at the temperature shown in Table 5 for 60 seconds on a hot plate. Thereafter, development was performed with the developer shown in Table 5 for 30 seconds to obtain a hole pattern with 32 nm in size.

Regarding the obtained resist pattern, the following evaluations were carried out. The results are shown in Table 5.

### [Sensitivity Evaluation]

The contact hole pattern was observed using a length-measurement SEM (CG-6300), manufactured by Hitachi High-Technologies Corporation, and an optimum exposure dose Eop (mJ/cm²) to yield the hole pattern with a size of 32 nm was determined.

### [Critical Dimension Uniformity (CDU) Evaluation]

Sizes of fifty hole patterns obtained by irradiation at the optimum exposure dose were measured, and a tripled value (3σ) of a standard variation (σ) calculated from the results was determined as CDU. A smaller CDU value can yield a pattern having a more uniform hole diameter.

### [Limiting Resolution Evaluation]

A hole pattern was formed while gradually decreasing the exposure dose from the optimum exposure dose at which the hole pattern can be formed, and in this event, the limit of the hole diameter (nm) at which resolution was possible was determined using a length-measurement SEM (CG-6300), manufactured by Hitachi High-Technologies Corporation, to specify this value as limiting resolution (nm). A smaller value indicates that it is possible to form a pattern having a finer hole diameter with better limiting resolution.

TABLE 5

| | | | | | | Limiting |
|---|---|---|---|---|---|---|
| | Resist | PAB/PEB | | Eop | CDU | resolution |
| | composition | (° C.) | Developer | (mJ/cm²) | (nm) | (nm) |
| Example 4-1 | R-01 | 130/90 | nBA | 15 | 1.8 | 17 |
| Example 4-2 | R-02 | 130/90 | nBA | 16 | 1.7 | 16 |
| Example 4-3 | R-03 | 130/90 | nBA | 11 | 1.8 | 19 |
| Example 4-4 | R-04 | 130/90 | nBA | 25 | 2.1 | 20 |
| Comparative | CR-01 | 130/90 | nBA | 36 | 3.2 | 26 |
| Example 4-1 | | | | | | |
| Comparative | CR-02 | 105/90 | TMAH | 42 | 3.8 | 32 |
| Example 4-2 | | | | | | |
| Comparative | CR-03 | 105/90 | TMAH | 40 | 4.0 | 32 |
| Example 4-3 | | | | | | |

From the results shown in Table 5, it was found that the inventive resist composition was excellent in sensitivity, CDU, and resolution in contact hole pattern formation by EUV exposure.

That is, from the above Examples, it was found that the inventive resist composition can achieve both high sensitivity and high resolution, and is extremely useful for forming a fine pattern in a patterning process.

It should be noted that the present invention is not limited to the above-described embodiments. The embodiments are just examples, and any examples that have substantially the same feature and demonstrate the same functions and effects as those in the technical concept disclosed in claims of the present invention are included in the technical scope of the present invention.

1. A resist composition comprising a hypervalent iodine compound represented by the following general formula (1) and a solvent, 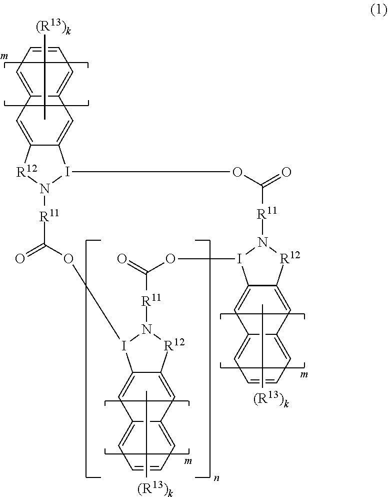
wherein “m” represents an integer of 0 to 2, “n” represents an integer of 1 to 40, and “k” represents an integer of 0 to 2m+4, the “m's” and the “k's” being identical to or different from each other;
each R¹¹ represents a carbonyl group or a hydrocarbylene group having 1 to 40 carbon atoms and optionally containing a heteroatom, the R¹¹'s being identical to or different from each other;
each R¹² represents a carbonyl group or a hydrocarbylene group having 1 to 10 carbon atoms and optionally containing a heteroatom, the R¹²'s being identical to or different from each other; and
each R¹³ represents a halogen atom or a hydrocarbyl group having 1 to 40 carbon atoms and optionally containing a heteroatom, the R¹³'s being identical to or different from each other.

2. A laminate comprising:
a substrate; and
a resist film obtained from the resist composition according to claim 1 on the substrate.

3. The laminate according to claim 2, further comprising a resist underlayer film between the substrate and the resist film.

4. A patterning process comprising the steps of:
forming a resist film by using the resist composition according to claim 1 on a substrate or on a resist underlayer film of a substrate on which the resist underlayer film has been laminated;
exposing the resist film by using a high-energy beam; and
developing the exposed resist film by using a developer.

5. The patterning process according to claim 4, wherein the high-energy beam used is an i-line, a KrF excimer laser beam, an ArF excimer laser beam, an electron beam, or an extreme ultraviolet ray.

6. The patterning process according to claim 4, wherein the developer used dissolves exposed portions and does not dissolve unexposed portions.

7. The patterning process according to claim 5, wherein the developer used dissolves exposed portions and does not dissolve unexposed portions.

8. The patterning process according to claim 4, wherein the developer used dissolves unexposed portions and does not dissolve exposed portions.

9. The patterning process according to claim 5, wherein the developer used dissolves unexposed portions and does not dissolve exposed portions.
